SEQUENCE LISTING (37 sequences)

SEQ ID NO: 1 - protein, 4 aa, Artificial Sequence
  Description of Artificial Sequence Illustrative peptide
AAPF

SEQ ID NO: 2 - DNA, 20 bp, Artificial Sequence
  Description of Artificial Sequence Primer
ataagattagcggatcctac

SEQ ID NO: 3 - DNA, 20 bp, Artificial Sequence
  Description of Artificial Sequence Primer
tcgggcatggcactcttgaa

SEQ ID NO: 4 - DNA, 1180 bp, Artificial Sequence
  Description of Artificial Sequence Synthetic subtilisin E
nnnnnnnnnnnnnnnngtgagaagcaaaaaattgtggatcagcttgttgtttgcgttaacgttaatctttacgatggcgttcagcaacatgtctgcgcaggctgccggaaaaagcagtacagaaaagaaatacattgtcggatttaaacagacaatgagtgccatgagttccgccaagaaaaaggatgttatttctgaaaaaggcggaaaggttcaaaagcaatttaagtatgttaacgcggccgcagcaacattggatgaaaaagctgtaaaagaattgaaaaaagatccgagcgttgcatatgtggaagaagatcatattgcacatgaatatgcgcaatctgttccttatggcatttctcaaattaaagcgccggctcttcactctcaaggctacacaggctctaacgtaaaagtagctgttatcgacagcggaattgactcttctcatcctgacttaaacgtcagaggcggagcaagcttcgtaccttctgaaacaaacccataccaggacggcagttctcacggtacgcatgtagccggtacgattgccgctcttaataactcaatcggtgttctgggcgttagcccaagcgcatcattatatgcagtaaaagtgcttgattcaacaggaagcggccaatatagctggattattaacggcattgagtgggccatttccaacaatatggatgttatcaacatgagccttggcggacctactggttctacagcgctgaaaacagtcgttgacaaagccgtttccagcggtatcgtcgttgctgccgcagccggaaacgaaggttcatccggaagcacaagcacagtcggctaccctgcaaaatatccttctactattgcagtaggtgcggtaaacagcagcaaccaaagagcttcattctccagcgcaggttctgagcttgatgtgatggctcctggcgtgtccatccaaagcacacttcctggaggcacttacggcgcttataacggaacgtccatggcgactcctcacgttgccggagcagcagcgttaattctttctaagcacccgacttggacaaacgcgcaagtccgtgatcgtttagaaagcactgcaacatatcttggaaactctttctactatggaaaagggttaatcaacgtacaagcagctgcacaataannnnnnnnnnnnnnnnnn

SEQ ID NO: 5 - DNA, 45 bp, Artificial Sequence
  Description of Artificial Sequence Synthetic oligonucleotide
ggatccnnnnnnnnnnnnnnnnnnnnnnnnnnnnnnnnnnnnnnc

SEQ ID NO: 6 - DNA, 42 bp, Artificial Sequence
  Description of Artificial Sequence Synthetic oligonucleotide
ggatccnnnnnnnnnnnnnnnnnnnnnnnnnnnnnnnnnnnc

SEQ ID NO: 7 - DNA, 39 bp, Artificial Sequence
  Description of Artificial Sequence Synthetic oligonucleotide
ggatccnnnnnnnnnnnnnnnnnnnnnnnnnnnnnnnnc

SEQ ID NO: 8 - DNA, 36 bp, Artificial Sequence
  Description of Artificial Sequence Synthetic oligonucleotide
ggatccnnnnnnnnnnnnnnnnnnnnnnnnnnnnnc

SEQ ID NO: 9 - DNA, 33 bp, Artificial Sequence
  Description of Artificial Sequence Synthetic oligonucleotide
ggatccnnnnnnnnnnnnnnnnnnnnnnnnnnc

SEQ ID NO: 10 - DNA, 30 bp, Artificial Sequence
  Description of Artificial Sequence Synthetic oligonucleotide
ggatccnnnnnnnnnnnnnnnnnnnnnnnc

SEQ ID NO: 11 - DNA, 27 bp, Artificial Sequence
  Description of Artificial Sequence Synthetic oligonucleotide
ggatccnnnnnnnnnnnnnnnnnnnnc

SEQ ID NO: 12 - DNA, 24 bp, Artificial Sequence
  Description of Artificial Sequence Synthetic oligonucleotide
ggatccnnnnnnnnnnnnnnnnnc

SEQ ID NO: 13 - DNA, 21 bp, Artificial Sequence
  Description of Artificial Sequence Synthetic oligonucleotide
ggatccnnnnnnnnnnnnnnc

SEQ ID NO: 14 - DNA, 18 bp, Artificial Sequence
  Description of Artificial Sequence Synthetic oligonucleotide
ggatccnnnnnnnnnnnc

SEQ ID NO: 15 - DNA, 15 bp, Artificial Sequence
  Description of Artificial Sequence Synthetic oligonucleotide
ggatccnnnnnnnnc

SEQ ID NO: 16 - DNA, 12 bp, Artificial Sequence
  Description of Artificial Sequence Synthetic oligonucleotide
ggatccnnnnnc

SEQ ID NO: 17 - DNA, 12 bp, Artificial Sequence
  Description of Artificial Sequence Synthetic oligonucleotide
nnnnnnnnnnnn

SEQ ID NO: 18 - DNA, 15 bp, Artificial Sequence
  Description of Artificial Sequence Synthetic oligonucleotide
nnnnnnnnnnnnnnn

SEQ ID NO: 19 - DNA, 18 bp, Artificial Sequence
  Description of Artificial Sequence Synthetic oligonucleotide
nnnnnnnnnnnnnnnnnn

SEQ ID NO: 20 - DNA, 21 bp, Artificial Sequence
  Description of Artificial Sequence Synthetic oligonucleotide
nnnnnnnnnnnnnnnnnnnnn

SEQ ID NO: 21 - DNA, 24 bp, Artificial Sequence
  Description of Artificial Sequence Synthetic oligonucleotide
nnnnnnnnnnnnnnnnnnnnnnnn

SEQ ID NO: 22 - DNA, 27 bp, Artificial Sequence
  Description of Artificial Sequence Synthetic oligonucleotide
nnnnnnnnnnnnnnnnnnnnnnnnnnn

SEQ ID NO: 23 - DNA, 30 bp, Artificial Sequence
  Description of Artificial Sequence Synthetic oligonucleotide
nnnnnnnnnnnnnnnnnnnnnnnnnnnnnn

SEQ ID NO: 24 - DNA, 33 bp, Artificial Sequence
  Description of Artificial Sequence Synthetic oligonucleotide
nnnnnnnnnnnnnnnnnnnnnnnnnnnnnnnnn

SEQ ID NO: 25 - DNA, 39 bp, Artificial Sequence
  Description of Artificial Sequence Synthetic oligonucleotide
nnnnnnnnnnnnnnnnnnnnnnnnnnnnnnnnnnnnnnc

SEQ ID NO: 26 - DNA, 39 bp, Artificial Sequence
  Description of Artificial Sequence Synthetic oligonucleotide
nnnnnnnnnnnnnnnnnnnnnnnnnnnnnnnnnnncnnn

SEQ ID NO: 27 - DNA, 39 bp, Artificial Sequence
  Description of Artificial Sequence Synthetic oligonucleotide
nnnnnnnnnnnnnnnnnnnnnnnnnnnnnnnncnnnnnn

SEQ ID NO: 28 - DNA, 39 bp, Artificial Sequence
  Description of Artificial Sequence Synthetic oligonucleotide
nnnnnnnnnnnnnnnnnnnnnnnnnnnnncnnnnnnnnn

SEQ ID NO: 29 - DNA, 39 bp, Artificial Sequence
  Description of Artificial Sequence Synthetic oligonucleotide
nnnnnnnnnnnnnnnnnnnnnnnnnncnnnnnnnnnnnn

SEQ ID NO: 30 - DNA, 39 bp, Artificial Sequence
  Description of Artificial Sequence Synthetic oligonucleotide
nnnnnnnnnnnnnnnnnnnnnnncnnnnnnnnnnnnnnn

SEQ ID NO: 31 - DNA, 39 bp, Artificial Sequence
  Description of Artificial Sequence Synthetic oligonucleotide
nnnnnnnnnnnnnnnnnnnncnnnnnnnnnnnnnnnnnn

SEQ ID NO: 32 - DNA, 39 bp, Artificial Sequence
  Description of Artificial Sequence Synthetic oligonucleotide
nnnnnnnnnnnnnnnnncnnnnnnnnnnnnnnnnnnnnn

SEQ ID NO: 33 - DNA, 39 bp, Artificial Sequence
  Description of Artificial Sequence Synthetic oligonucleotide
nnnnnnnnnnnnnncnnnnnnnnnnnnnnnnnnnnnnnn

SEQ ID NO: 34 - DNA, 39 bp, Artificial Sequence
  Description of Artificial Sequence Synthetic oligonucleotide
nnnnnnnnnnncnnnnnnnnnnnnnnnnnnnnnnnnnnn

SEQ ID NO: 35 - DNA, 39 bp, Artificial Sequence
  Description of Artificial Sequence Synthetic oligonucleotide
nnnnnnnncnnnnnnnnnnnnnnnnnnnnnnnnnnnnnn

SEQ ID NO: 36 - DNA, 39 bp, Artificial Sequence
  Description of Artificial Sequence Synthetic oligonucleotide
nnnnncnnnnnnnnnnnnnnnnnnnnnnnnnnnnnnnnn

SEQ ID NO: 37 - DNA, 39 bp, Artificial Sequence
  Description of Artificial Sequence Synthetic oligonucleotide
nncnnnnnnnnnnnnnnnnnnnnnnnnnnnnnnnnnnnn

(19) United States
(12) Patent Application Publication
(10) Pub. No.: US 2006/0068406 A1
(43) Pub. Date: Mar. 30, 2006

(54) Single-stranded nucleic acid template-mediated recombination and nucleic acid fragment isolation

(75) Inventors: Joseph A. Affholter, Zephyr Cove, NV (US); Anthony Cox, Mountain View, CA (US); Jon E. Ness, Redwood City, CA (US); Brian Carr, Raleigh, NC (US)

Correspondence Address: MAXYGEN, INC.;INTELLECTUAL PROPERTY DEPARTMENT, 515 GALVESTON DRIVE, RED WOOD CITY, CA, 94063, US

(73) Assignee: Maxygen, Inc.

(21) Appl. No.: 11/047,380

(22) Filed: Jan. 31, 2005

Related U.S. Application Data
(63) Continuation of application No. 09/721,507, filed on Nov. 22, 2000, now abandoned.
Application No. 09/721,507 is a continuation of application No. 09/656,549, filed on Sep. 6, 2000, now abandoned.
(60) Provisional application No. 60/185,244, filed on Feb. 28, 2000.
(60) Provisional application No. 60/185,815, filed on Feb. 29, 2000.
(60) Provisional application No. 60/186,247, filed on Mar. 1, 2000.
(60) Provisional application No. 60/186,482, filed on Mar. 2, 2000.

(51) Int. Cl. C12Q 1/68; C12P 19/34

(52) U.S. Cl. 435/6; 435/91.1

(57) ABSTRACT

Methods mediated by single-stranded nucleic acid templates, including utilizing single-stranded nucleic acid templates to isolate nucleic acid fragments and to recombine nucleic acid fragments. Methods include polymerase and polymerase-free recombination of nucleic acid fragments to generate chimeric nucleic acid sequences. Integrated systems and kits are also provided.

### CROSS REFERENCE TO RELATED APPLICATIONS

Pursuant to 35 USC 119 and/or 120, and any other applicable statute or rule, this application claims the benefit of and priority to each of the following Application Numbers/filing dates: U.S. Ser. No. 60/185,244, filed Feb. 28, 2000; U.S. Ser. No. 60/185,815, filed Feb. 29, 2000; U.S. Ser. No. 60/186,247, filed Mar. 1, 2000; and U.S. Ser. No. 60/186,482, filed Mar. 2, 2000, the disclosures of which are incorporated by reference.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Nucleic acid recombination methodologies, such as iterative nucleic acid shuffling approaches represent landmark advances in the access of sequence space. The inventor and co-workers have developed various rapid artificial evolution techniques that provide superior agriculturally, industrially, and pharmaceutically relevant genes and expression products. These methodologies and related aspects are described in a variety of sources, e.g., Stemmer et al., (1994) “Rapid Evolution of a Protein” Nature 370:389-391, Stemmer (1994) “DNA Shuffling by Random Fragmentation and Reassembly: in vitro Recombination for Molecular Evolution,” Proc. Natl. Acad. USA 91:10747-10751, Crameri et al., (1996), “Construction And Evolution Of Antibody-Phage Libraries By DNA Shuffling” Nature Medicine 2(1):100-103, Stemmer U.S. Pat. No. 5,605,793 “METHODS FOR IN VITRO RECOMBINATION,” Stemmer et al., U.S. Pat. No. 5,830,721 “DNA MUTAGENESIS BY RANDOM FRAGMENTATION AND REASSEMBLY,” Stemmer et al., U.S. Pat. No. 5,811,238 “METHODS FOR GENERATING POLYNUCLEOTIDES HAVING DESIRED CHARACTERISTICS BY ITERATIVE SELECTION AND RECOMBINATION,” Stemmer et al., (1998) U.S. Pat. No. 5,834,252 “END-COMPLEMENTARY POLYMERASE REACTION,” Minshull et al., U.S. Pat. No. 5,837,458 “METHODS AND COMPOSITIONS FOR CELLULAR AND METABOLIC ENGINEERING,” and PCT/US 00/01203 “OLIGONUCLEOTIDE MEDIATED NUCLEIC ACID RECOMBINATION,” filed Jan. 18, 2000, each of which is incorporated by reference in its entirety for all purposes. Additional details regarding DNA shuffling can also be found in WO95/22625, WO97/20078, WO96/33207, WO97/33957, WO98/27230, WO97/35966, WO98/31837, WO98/13487, WO98/13485 and WO98/42832, each of which is also incorporated by reference in its entirety for all purposes.

Additional recombination methods would be desirable. The present invention provides methods of single-stranded nucleic acid template-mediated recombination and nucleic acid fragment isolation, as well as a variety of additional features which will become apparent upon review of the following description.

### SUMMARY OF THE INVENTION

The present invention relates to various recombination methods mediated, e.g., by single-stranded nucleic acid template assembly. The methods include, e.g., utilizing single-stranded nucleic acid templates to isolate nucleic acid fragments. The invention also provides nucleic acid fragment recombination methods that involve single-stranded templates, including, e.g., polymerase and polymerase-free (e.g., ligase-mediated) nucleic acid recombination.

The invention provides methods of recombining a set of nucleic acid fragments. The methods include hybridizing at least two sets of nucleic acids, e.g., a first set of nucleic acids that includes single-stranded nucleic acid templates and a second set of nucleic acids that includes the set of nucleic acid fragments. Optionally, the set of single-stranded templates is at least substantially either all sense strands or all antisense strands, and the nucleic acid fragments (in the set of nucleic acid fragments) are at least substantially all single-stranded and derived from the opposite strand of those employed in the set of single-stranded templates (e.g., if single-stranded sense templates are used, then single-stranded antisense fragments are used). Additionally, the methods optionally include removing nonhybridizing portions of partially hybridized fragments, elongating, ligating, or both, sequence gaps between hybridized nucleic acid fragments to generate at least substantially full-length chimeric nucleic acid sequences that correspond to the single-stranded nucleic acid templates to recombine the set of nucleic acid fragments.

The first set of nucleic acids (e.g., single-stranded nucleic acid templates) can include, e.g., sense cDNA sequences, antisense cDNA sequences, sense DNA sequences, antisense DNA sequences, sense RNA sequences, antisense RNA sequences, natural sequences, artificial sequences, mutant sequences, recombined sequences or the like. Each single-stranded nucleic acid template also optionally includes at least one affinity-label. Furthermore, the first and second sets of nucleic acids optionally include substantially homologous sequences. Optionally, the first set of nucleic acids is synthesized.

The present invention includes many different options for providing the second set of nucleic acids (e.g., the nucleic acid fragments) used in the methods herein. For example, the second set of nucleic acids can alternately include a standardized or a non-standardized set of nucleic acids. The second set of nucleic acids can also include chimeric nucleic acid sequence fragments derived from, e.g., chimeric sequences generated by the nucleic acid recombination methods of the present invention. Additionally, the second set of nucleic acids can be derived from, e.g., cultured microorganisms, uncultured microorganisms, complex biological mixtures, tissues, sera, pooled sera or tissues, multispecies consortia, fossilized or other nonliving biological remains, environmental isolates, soils, groundwaters, waste facilities, deep-sea environments, or the like. The second set of nucleic acids can also be derived from, e.g., individual cDNA molecules, cloned sets of cDNAs, cDNA libraries, extracted RNAs, natural RNAs, in vitro transcribed RNAs, characterized genomic DNAs, uncharacterized genomic DNAs, cloned genomic DNAs, genomic DNA libraries, enzymatically fragmented DNAs, enzymatically fragmented RNAs, chemically fragmented DNAs, chemically fragmented RNAs, physically fragmented DNAs, physically fragmented RNAs, or the like. Another option includes synthesizing the second set of nucleic acids. Optionally, the first set of nucleic acids (e.g., the single-stranded nucleic acid templates) is also derived from the same sources as the second set of nucleic acids. The first and second sets of nucleic acids can also be derived from different sets of nucleic acids.

The methods of recombining a set of nucleic acid fragments optionally include cleaving unhybridized portions of the hybridized nucleic acid fragments (e.g., by nuclease cleavage or the like) prior to performing the elongating or ligating step. Further, the methods also optionally include separating hybridized nucleic acids from unhybridized nucleic acids by a separation technique before or after performing the cleaving step (e.g., chemically, enzymatically, via physical strand separation, or the like). The methods optionally include denaturing the at least substantially full-length chimeric nucleic acid sequences and the single-stranded nucleic acid templates. The at least substantially full-length chimeric nucleic acid sequences can also be separated from the single-stranded nucleic acid templates by a separation technique. Thereafter, the separated at least substantially full-length chimeric nucleic acid sequences can be fragmented by, e.g., nuclease digestion or physical fragmentation to provide chimeric nucleic acid sequence fragments that can optionally be included, e.g., as substrates for additional recombination.

Separation techniques used in these methods can include any of various techniques or technique combinations including, e.g., an affinity-based separation, centrifugation, fluorescence-based separation, magnetic field-based separation, electrophoretic separation, fluidic molecular separation, microfluidic molecular separation, chromatographic separation, or the like.

The present invention also includes methods of isolating nucleic acid fragments from a set of nucleic acid fragments. The methods include, e.g., hybridizing at least two sets of nucleic acids, e.g., a first set of nucleic acids that includes single-stranded nucleic acid templates and a second set of nucleic acids that includes the set of nucleic acid fragments. The methods can also include separating the hybridized nucleic acids from unhybridized nucleic acids by at least one first separation technique and denaturing the separated hybridized nucleic acids to yield the single-stranded nucleic acid templates and isolated nucleic acid fragments. Optionally, the methods include separating the isolated nucleic acid fragments from the single-stranded nucleic acid templates by at least one second separation technique following the denaturing step. The first and second separation techniques can be selected from, e.g., an affinity-based separation, a centrifugation, a fluorescence-based separation, a magnetic field-based separation, an electrophoretic separation, a microfluidic molecular separation, a magnetic separation, a chromatographic separation, and the like. The isolated nucleic acid fragments can optionally be included, e.g., as substrates for the various methods of recombining nucleic acids described herein.

As with the methods of recombining nucleic acid fragments, described above, the first set of nucleic acids (e.g., the single-stranded nucleic acid templates), used in the methods of isolating nucleic acid fragments, can include, e.g., sense cDNA sequences, antisense cDNA sequences, sense DNA sequences, antisense DNA sequences, sense RNA sequences, antisense RNA sequences, natural sequences, artificial sequences, and/or the like. The first set of nucleic acids can be isolated, synthesized or produced by any other available method. Additionally, the single-stranded nucleic acid templates can each include at least one affinity-label. Optionally, the first and second sets of nucleic acids can include substantially homologous sequences and either may be optionally interrupted (or interspersed) by naturally occurring or synthetic introns or other intervening sequences which disrupt the intended open-reading frame.

The methods of isolating nucleic acid fragments optionally include providing the single-stranded nucleic acid templates to include sense single-stranded nucleic acid templates and the set of nucleic acid fragments to include a set of antisense nucleic acid fragments that correspond to the sense single-stranded nucleic acid templates to provide isolated antisense nucleic acid fragments. Alternatively, the methods can include providing the single-stranded nucleic acid templates to include antisense single-stranded nucleic acid templates and the set of nucleic acid fragments to include a set of sense nucleic acid fragments that correspond to the antisense single-stranded nucleic acid templates to provide isolated sense nucleic acid fragments. The isolated sense and antisense nucleic acid fragment populations can subsequently be used as substrates in various downstream processing steps.

The second set of nucleic acids (e.g., the nucleic acid fragments) used in the methods of isolating nucleic acid fragments can also be derived from various alternative sources. For example, the second set of nucleic acids can optionally include a standardized or a non-standardized set of nucleic acids. The second set of nucleic acids also optionally includes chimeric nucleic acid sequence fragments Additionally, the second set of nucleic acids can be derived from, e.g., cultured microorganisms, uncultured microorganisms, complex biological mixtures, tissues, sera, pooled sera or tissues, multispecies consortia, fossilized or other nonliving biological remains, environmental isolates, soils, groundwaters, waste facilities, deep-sea environments, or the like. The second set of nucleic acids can also be derived from, e.g., individual cDNA molecules, cloned sets of cDNAs, cDNA libraries, extracted RNAs, natural RNAs, in vitro transcribed RNAs, characterized genomic DNAs, uncharacterized genomic DNAs, cloned genomic DNAs, genomic DNA libraries, enzymatically fragmented DNAs, enzymatically fragmented RNAs, chemically fragmented DNAs, chemically fragmented RNAs, physically fragmented DNAs, physically fragmented RNAs, or the like. An additional option includes synthesizing the second set of nucleic acids. Optionally, the first set of nucleic acids (e.g., the single-stranded nucleic acid templates) is also derived from the same sources as the second set of nucleic acids.

The methods of the present invention can include performing each step sequentially in a single reaction vessel. Optionally, at least one step of the methods can be performed in a reaction vessel separate from other steps.

The methods of the invention include various other alternative steps. For example, unhybridized portions of the hybridized nucleic acid fragments can be cleaved by nuclease cleavage before or after the separating step. This step (i.e., removal of unhybridized, single-stranded fragments) can be followed by elongating, ligating, or both, sequence gaps between hybridized nucleic acid fragments to generate at least substantially full-length chimeric nucleic acid sequences that correspond to the single-stranded nucleic acid templates. Complementary strand synthesis (e.g., with an oligonucleotide primer) of the at least substantially full-length chimeric nucleic acid sequences and amplification can optionally be conducted (with or without prior separation of the assembled chimeric nucleic acid sequences from the single-stranded templates). Additionally, the at least one amplified at least substantially full-length chimeric nucleic acid sequence can be selected for a desired trait, such as by detection of a physical or chemical (e.g., binding, catalytic, fluorometric, and the like) property of an encoded expression product. A further option includes, fragmenting the amplified at least substantially full-length chimeric nucleic acid sequences by nuclease digestion or physical fragmentation to provide chimeric nucleic acid sequence fragments. The chimeric nucleic acid sequence fragments can then be used, e.g., as substrates for the methods of recombining a set of nucleic acid fragments, as substrates for the methods of isolating a set of nucleic acids fragments, or the like.

The present invention also includes methods of providing a population of recombined nucleic acids. The methods can include hybridizing the isolated nucleic acid fragments or the chimeric nucleic acid sequence fragments. Optionally, isolated sense and antisense nucleic acid fragments can be hybridized. In this case, the isolated nucleic acid fragments include isolated sense and antisense nucleic acid fragments in which the isolated sense nucleic acid fragments correspond to the isolated antisense nucleic acid fragments. Thereafter, the hybridized isolated nucleic acid fragments or the hybridized chimeric nucleic acid sequence fragments can be elongated or ligated, e.g., to provide a population of recombined nucleic acids.

The methods also optionally include introducing one or more members of the population of recombined nucleic acids into a cell. Additionally, the one or more introduced members of the population of recombined nucleic acids can be expressed to provide an expression product to the cell. The methods can also optionally include expressing the population of recombined nucleic acids (e.g., in vitro) to provide an expression product that can be selected for a desired trait or property.

The population of recombined nucleic acids can also be further recombined, e.g., to generate additional diversity. The methods can include denaturing (i.e., the second denaturing step) the population of recombined nucleic acids, rehybridizing the denatured population of recombined nucleic acids, and extending the rehybridized population of recombined nucleic acids to provide a population of further recombined nucleic acids. Optionally, the second denaturing, rehybridizing, and extending steps can be repeated at least once.

In one aspect, the invention provides methods of recombining a set of nucleic acid fragments. The method includes, e.g., hybridizing at least two sets of nucleic acids, where a first set of nucleic acids comprises single-stranded sense strand-nucleic acid templates and a second set of nucleic acids consists essentially of single-stranded antisense strand-nucleic acid fragments. Typically, the method further includes elongating, ligating, or both elongating and ligating sequence gaps between the hybridized nucleic acid fragments to generate at least substantially full-length chimeric nucleic acid sequences that correspond to the single-stranded nucleic acid templates, thereby recombining the set of nucleic acid fragments.

In an alternate aspect, the methods include hybridizing at least two sets of nucleic acids, where a first set of nucleic acids comprises single-stranded antisense strand-nucleic acid templates and a second set of nucleic acids consists essentially of single-stranded sense strand-nucleic acid fragments. In this aspect, the methods also include elongating, ligating, or both, sequence gaps between the hybridized nucleic acid fragments to generate at least substantially full-length chimeric nucleic acid sequences that correspond to the single-stranded nucleic acid templates, thereby recombining the set of nucleic acid fragments.

In an alternate aspect, the methods include hybridizing at least two sets of nucleic acids, where a first set of nucleic acids includes single-stranded nucleic acid templates and a second set of nucleic acids includes at least one set of nucleic acid fragments. In this aspect, the methods include elongating, ligating, or both, sequence gaps between the hybridized nucleic acid fragments by incubating the hybridized nucleic acid fragments with a polymerase and/or a ligase at a temperature of about 45° C. or less (e.g., 37° C. or less or e.g., 25° C. or less), to generate at least substantially full-length chimeric nucleic acid sequences that correspond to the single-stranded nucleic acid templates, thereby recombining the set of nucleic acid fragments.

In one aspect, the invention provides methods of recombining a set of nucleic acid fragments in which a set of at least partially double-stranded nucleic acids that encode a polypeptide of interest or portion thereof are provided. The set of at least partially double-stranded nucleic acids is contacted with an exonuclease that selectively degrades one strand of the at least partially double-stranded nucleic acids to provide a set of single-stranded nucleic acid templates. The set of single-stranded nucleic acid templates is hybridized with a second set of nucleic acids comprising at least one set of nucleic acid fragments. Sequence gaps are filled by elongation, ligation or both between the hybridized nucleic acid fragments to generate at least substantially full-length chimeric nucleic acid sequences that correspond to the single-stranded nucleic acid templates, thereby recombining the set of nucleic acid fragments.

In another aspect, the invention includes recombining a set of nucleic acid fragments by hybridizing at least two sets of nucleic acids. A first set of nucleic acids includes single-stranded nucleic acid templates and a second set of nucleic acids includes at least one set of nucleic acid fragments. The fragments are elongated, ligated, or both, to generate at least substantially full-length chimeric nucleic acid sequences that correspond to the single-stranded nucleic acid templates. The method further includes introducing one or more of the at least substantially full-length chimeric nucleic acid sequences into at least one cell, expressing the one or more introduced at least substantially full-length chimeric nucleic acid sequences to provide at least one expression product to the at least one cell, and selecting or screening the at least one cell for one or more desired traits or properties using at least one plate-based or at least one filter-based assay.

In one aspect, the invention provides a method of combinatorially assembling nucleic acids. The method includes hybridizing at least two sets of nucleic acids, where a first of the at least to sets of nucleic acids includes single-stranded nucleic acid templates and a second set of the at least two sets of nucleic acids includes at least one set of nucleic acid fragments. The fragments hybridize to a plurality of subsequences on at least one member of the first set of nucleic acids, where hybridization of the first and second set of nucleic acids directs combinatorial assembly of a third set nucleic acids. The first and second set of nucleic acids are optionally transduced into one or more cells in hybridized form, whereby the cells produce the third set of nucleic acids. The first and second set of nucleic acids are optionally transduced into the cell following treatment a polymerase, a ligase or an exonuclease. Alternately, the first and second set of nucleic acids are transduced into the cell without treatment by the polymerase, ligase or exonuclease. The first or second set of nucleic acids are optionally homologous (e.g., derived from one or more related sequences, e.g., allelic, species or artificially produced variants. Optionally in this class of methods, the hybridized first and second sets of nucleic acids can be incubated with a nuclease, a ligase or a polymerase. The hybridized first and second set of nucleic acids optionally provide one or more overlapping sets of nucleic acids. As with many other methods herein, the recombination methods optionally further include selecting or screening one or more members of the third set of nucleic acids for one or more traits or properties of encoded expression products.

In one aspect, the invention provides methods of recombining a set of nucleic acid fragments. As with several of the methods above, the method includes hybridizing at least two sets of nucleic acids. In this embodiment, a first set of nucleic acids comprises single-stranded sense strand-nucleic acid templates and a second set of nucleic acids consists essentially of single-stranded antisense strand-nucleic acid fragments. The fragments are elongated, ligated, or both, to fill sequence gaps between the hybridized nucleic acid fragments to generate at least substantially full-length chimeric nucleic acid sequences. These sequences correspond to the single-stranded nucleic acid templates.

In a similar aspect, the invention provides a method of recombining a set of nucleic acid fragments, in which at least two sets of nucleic acids are hybridized and where a first set of nucleic acids includes single-stranded antisense strand-nucleic acid templates and a second set of nucleic acids consists essentially of single-stranded sense strand-nucleic acid fragments elongated, ligated, or both, to fill sequence gaps between the hybridized nucleic acid fragments to generate at least substantially full-length chimeric nucleic acid sequences.

In an alternate embodiment, the invention provides methods of recombining a set of nucleic acid fragments. In this class of recombination methods a set of at least partially double-stranded nucleic acids that encode a polypeptide of interest or portion thereof is provided. The set of at least partially double-stranded nucleic acids is contacted with an exonuclease that selectively degrades one strand of the at least partially double-stranded nucleic acids to provide a set of single-stranded nucleic acid templates. The set of single-stranded nucleic acid templates hybridizes with a second set of nucleic acids comprising at least one set of nucleic acid fragments. The fragments are elongated, ligated, or both to fill/join sequence gaps between the hybridized nucleic acid fragments to generate at least substantially full-length chimeric nucleic acid sequences that correspond to the single-stranded nucleic acid templates. Common exonucleases for this purpose include Exonuclease III, Bal31, Mung bean nuclease, T7 gene 6 exonuclease, and lambda exonuclease. The nucleic acid fragments are single stranded or double stranded.

In one aspect, the methods noted above include introducing one or more of the at least substantially full-length chimeric nucleic acid sequences into at least one cell, expressing the one or more introduced at least substantially full-length chimeric nucleic acid sequences to provide at least one expression product to the at least one cell, and, selecting or screening the at least one cell for one or more desired traits or properties using at least one plate-based or at least one filter-based assay.

Definitions

Unless otherwise indicated, the following definitions supplement those in the art.

An “amplicon” is a nucleic acid made using the polymerase chain reaction (PCR). Typically, the nucleic acid is a copy of a selected nucleic acid. A “primer” is a nucleic acid which hybridizes to a template nucleic acid and permits chain elongation using, e.g., a thermostable polymerase under appropriate reaction conditions.

A “chimeric” nucleic acid sequence can include a sequence composed of nucleic acid subsequences derived from different sources, e.g., nucleic acid fragments from different genes, different organisms, and the like. An “at least substantially full-length chimeric nucleic acid sequence” can include, e.g., a recombined set of nucleic acid fragments that is complementary, or partially complimentary e.g., to substantially the full-length of a single-stranded nucleic acid template.

Two nucleic acids “correspond” when they have the same sequence, or when one nucleic acid is complementary to the other, or when one nucleic acid is a subsequence of the other, or when one sequence is derived, by natural or artificial manipulation from the other.

Nucleic acids are “elongated” in a reaction that incorporates additional nucleotides, or analogs thereof, into the nucleic acid sequence. For example, a sequence gap is elongated when additional nucleotides, or analogs thereof, are added to one or both nucleic acid fragments hybridized to either side of the sequence gap. The reaction is typically catalyzed by a polymerase, e.g., a DNA polymerase, an RNA polymerase, and the like. Nucleic acid fragments are “ligated” or joined together in a reaction typically catalyzed by, e.g., a ligase or by an enzyme having ligase activity (e.g., which catalyzes formation of phospohdiester linkages between 3′ and 5′ positions of nucleic acids and nucleic acid analogs). For example, a sequence gap is ligated when nucleic acid fragments hybridized to either side of the sequence gap are joined together, e.g., directly (e.g., in a polymerase-free embodiment of the invention), following sequence gap elongation (e.g., with a polymerase), or the like.

A set of “fragmented” nucleic acids results from the cleavage of at least one parental nucleic acid, e.g., physically (e.g., by shearing, sonication, or the like), enzymatically (e.g., by nuclease digestion, such as an RNAse, a DNAse, an exonuclease, an endonuclease, or the like), or chemically, or by providing subsequences of parental sequences in any other manner, including partially elongating a complementary sequence with a polymerase or utilizing any synthetic format.

Nucleic acids are “homologous” when they share sequence similarity that is derived, naturally or artificially, from a common ancestral sequence. This occurs naturally as two or more descendent sequences deviate from a common ancestral sequence over time as the result of mutation and natural selection. Artificially homologous sequences may be generated in various ways. For example, a nucleic acid sequence can be synthesized de ovo to yield a nucleic acid that differs in sequence from a selected parental nucleic acid sequence. Artificial homology can also be created by artificially recombining one nucleic acid sequence with another, as occurs, e.g., during cloning or chemical mutagenesis, to produce a homologous descendent nucleic acid. Artificial homology may also be created using the redundancy of the genetic code to synthetically adjust some or all of the coding sequences between otherwise dissimilar nucleic acids in such a way as to increase the frequency and length of highly similar stretches of nucleic acids while minimizing resulting changes in amino acid sequence to the encoded gene products. Preferably, such artificial homology is directed to increasing the frequency of identical stretches of sequence of at least three base pairs in length. More preferably, it is directed to increasing the frequency of identical stretches of sequence of at least four base pairs in length.

It is generally assumed that the two nucleic acids have common ancestry when they demonstrate sequence similarity. However, the exact level of sequence similarity necessary to establish homology varies in the art. In general, for purposes of this disclosure, two nucleic acid sequences are deemed to be homologous when they share enough sequence identity to permit direct recombination to occur between the two sequences.

Nucleic acids “hybridize” when they associate, typically in solution (or with one component fixed to a solid support). Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2 “Overview of principles of hybridization and the strategy of nucleic acid probe assays,” (Elsevier, N.Y.), as well as Current Protocols in Molecular Biology, F. M. Ausubel et al., eds., Current Protocols, a joint venture between Greene Publishing Associates, Inc. and John Wiley & Sons, Inc., (1999 Supplement). Hames and Higgins (1995) Gene Probes 1 IRL Press at Oxford University Press, Oxford, England, and Hames and Higgins (1995) Gene Probes 2 IRL Press at Oxford University Press, Oxford, England provide details on the synthesis, labeling, detection and quantification of DNA and RNA, including oligonucleotides.

A “nucleic acid” is a deoxyribonucleotide or ribonucleotide polymer in either single- or double-stranded form, and unless otherwise limited, encompasses known analogs of natural nucleotides that function in a manner similar to naturally occurring nucleotides.

Two nucleic acids “recombine” when sequences or subsequences from each of the two nucleic acids are combined in a progeny nucleic acid.

A “sense” strand (or, coding (+) strand) includes the same nucleotide sequence as that of, e.g., an RNA transcript (e.g., an mRNA), except in the case of DNA where thymine bases replace uracil bases. An “antisense” strand (or, template (−) strand) is the complement of the RNA transcript.

A “sequence gap” is a region of a nucleic acid duplex in which one strand of the duplex lacks complementary nucleotides in the other strand. For example, following hybridization of a set of nucleic acid fragments to a single-stranded nucleic acid template, regions of the template strand can lack complementary nucleotides, e.g., between hybridized nucleic acid fragments, such that sequence gaps in the strand of the duplex that includes the nucleic acid fragments exist.

A “set” refers to a collection of at least two molecule or sequence types, e.g., 2, 3, 4, 5, 10, 20, 50, 100, 1,000 or more molecule or sequence types.

A “single-stranded nucleic acid template” can include, e.g., a single-stranded sequence of RNA, cDNA, DNA, and the like. The sequence can include a sense sequence, an antisense sequence, and the like.

A “standardized” set of nucleic acids includes a population where each member is uniformly or otherwise non-randomly represented. A “non-standardized” set of nucleic acids includes a random or naturally occurring collection of nucleic acids.

### BRIEF DESCRIPTION OF THE DRAWING

FIG. 1 schematically shows one embodiment of the methods of single-strand nucleic acid template-mediated recombination.

FIG. 2 schematically depicts certain embodiments of the methods of single-strand nucleic acid template-mediated recombination and nucleic acid fragment isolation including affinity labels.

FIG. 3 schematically shows one embodiment of the methods of single-strand nucleic acid template mediated recombination involving Ung-End template fragmentation.

FIG. 4 schematically illustrates one embodiment of the methods of creating chimeric nucleic acids by Mung bean nuclease-mediated heteroduplex repair.

FIG. 5 schematically depicts one embodiment of the methods of creating chimeric nucleic acids by uracil glycosylase-mediated heteroduplex repair.

FIG. 6 shows the nucleic acid sequence corresponding to subtilisin E.

FIG. 7A shows a population for incorporating invariant recombination and digestion sites.

FIG. 7B provides a population of staggered, non-redundant filler oligonucleotides.

FIG. 8 shows oligonucleotides constructed as single stranded combinatorial mutagenic cassettes.

### DETAILED DISCUSSION OF THE INVENTION

Single-stranded templates of RNA or DNA can be used to “order” or “orchestrate” the relative positioning of single-stranded nucleic acid fragments derived from standardized or non-standardized pools of nucleic acids. This strategy can be utilized to isolate or copurify specific nucleic acid fragments from a fragment population. For example, nucleic acid fragments with sequences or subsequences complementarity to a single-stranded template can be hybridized and separated from nonhybridizing nucleic acid fragments in the population. Thereafter, the hybridized fragments can be purified further by being separated from the single-stranded templates to which they hybridized to yield isolated nucleic acid fragments. The isolated nucleic acid fragments can, in turn, be used as substrates in various downstream processing steps, including, e.g., ligation, amplification, recombination, transformation, expression, selection, and the like.

Aside from fragment isolation, single-stranded nucleic acid templates can also be used to mediate various recombination methods. For example, sequences gaps between hybridized nucleic acid fragments that hybridize to a single-stranded template can be filled either by elongation and ligation steps or, if the fragments and the template share sufficient homology, by ligation alone. The resultant chimeric nucleic acid sequences, or full-length genes, are optionally subsequently denatured and separated from the template strands. The chimeric nucleic acid sequences can similarly be subject to assorted downstream processes. Alternatively, chimeric/template duplexes are transformed directly into appropriate expression hosts. The present invention provides these and many variations upon these methods of template-based nucleic acid recombination.

The following provides details regarding various aspects of the methods of single-stranded nucleic acid template-mediated nucleic acid fragment isolation and recombination. It also provides details pertaining to the sources and preparation of single-stranded templates and nucleic acid fragments. Furthermore, the following description also describes various downstream processing steps, integrated systems which model or assist in the recombination methods (or which act as upstream or downstream processes for sequence recombination), and kits related to the present invention.

Single-Stranded Nucleic Acid Template-Mediated Nucleic Acid Fragment Isolation

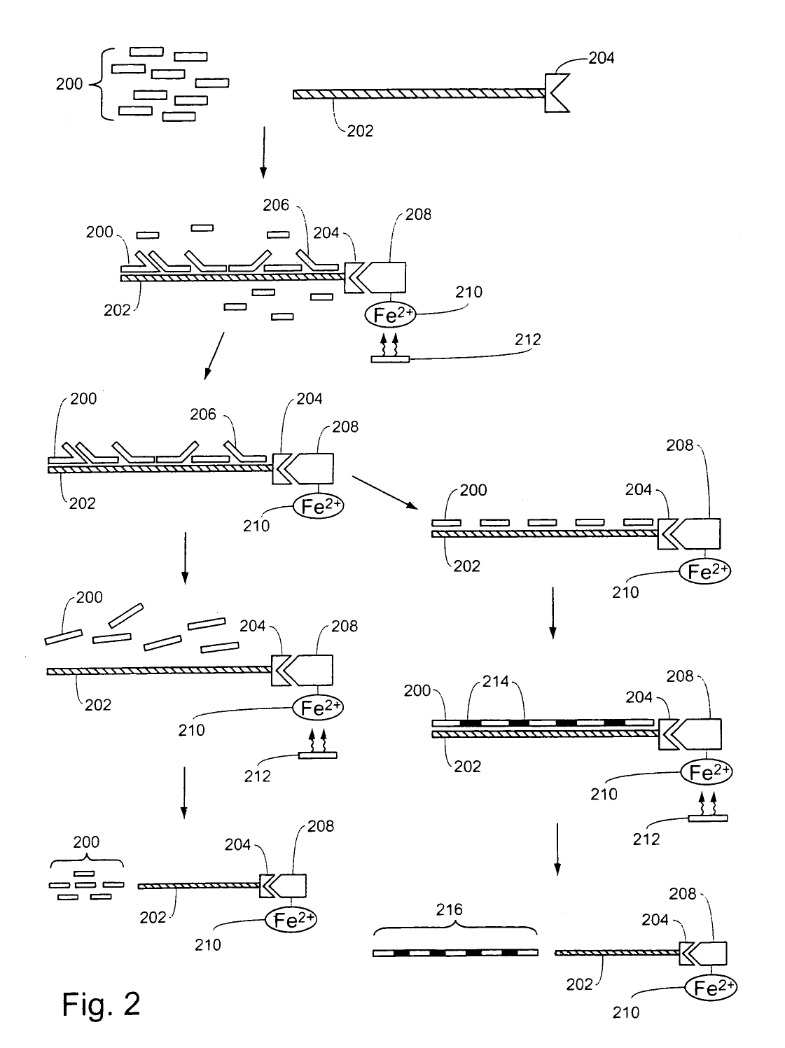

The present invention provides methods of isolating a set of nucleic acid fragments. One embodiment of these methods is schematically illustrated in the sequence of steps that concludes on the left-hand side of FIG. 2. As shown, the methods include, e.g., hybridizing at least two sets of nucleic acids, e.g., a first set of nucleic acids can include single-stranded nucleic acid template 202 which can optionally include affinity label 204 (e.g., biotin, digoxigenin, digoxin, a hybridization “tag” or “tail” or the like) and a second set of nucleic acids that includes nucleic acid fragments 200. Depending on the level of homology between single-stranded nucleic acid template 202 and nucleic acid fragments 200, the entire length of some fragments can substantially hybridize, while other hybridized fragments can include one or more unhybridized portions 206. As depicted, fragments lacking complementarity to single-stranded nucleic acid template 202 remain unbound.

As mentioned above, nucleic acids hybridize when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993), supra, and in Hames and Higgins, 1 and 2, supra. One of skill can easily determine appropriate hybridization reaction conditions for association of any two nucleic acids of interest, e.g., by increasing or decreasing stringency of hybridization (e.g., by increasing or decreasing salt or temperature parameters) and by monitoring hybridization. Once appropriate hybridization conditions are identified for association of template nucleic acids and bound nucleic acids, the conditions are used in the relevant methods.

The methods of the present invention can also include separating the hybridized nucleic acids from unhybridized nucleic acids by various well-known separation techniques, including affinity-based separation, a centrifugation, fluorescence-based separation, magnetic field-based separation, electrophoretic separation, microfluidic molecular separation, magnetic separation, chromatographic separation, and the like. As shown in FIG. 2, a preferred separation method can include binding a detector or capture complex that includes binding agent 208 linked to magnetic bead or other binding agent substrate 210. Although shown as a ferrous bead, a variety of other substrates can be substituted, including plastic particles, polymer particles, glass particles or the like. These can be separated from surrounding materials using any available technique, including magnetic field-based separation, centrifugation, density sedimentation, affinity-based separation, or the like. Suitable binding agents (e.g., avidin, streptavidin, anti-digoxigenin, and the like) linked to magnetic beads are readily available from various commercial sources, such as from Dynal AS (www.dynal.no). Single-stranded nucleic acid template 202 with hybridized nucleic acid fragments 200 can be, e.g., captured by applying magnetic field 212 which acts on magnetic bead 210. Upon capture, unhybridized fragments can, e.g., be washed away leaving the captured hybridized complexes. As a further option, either before or after separating hybridized from unhybridized fragments, one or more unhybridized portions 206 can be cleaved by nuclease digestion (e.g., an exonuclease). Note, also that either before or after this separation step, the hybridized fragments are optionally recombined according to various methods described in greater detail below (i.e., single-strand nucleic acid template-mediated recombination). Following recombination, the recombined nucleic acid fragments are also optionally subject to downstream processing steps that are also discussed further below.

Following the separation of the hybridized fragments from the unhybridized fragments, hybridized nucleic acid fragments 200 are optionally separated from single-stranded nucleic acid template 202 by denaturing nucleic acid fragments 200 (e.g., by applying heat, etc.) while maintaining the capture of single-stranded nucleic acid template 202 in magnetic field 212. Other separation techniques, such as those mentioned above can also optionally be used. As shown in FIG. 2, this method ultimately yields an isolated set of nucleic acid fragments that were initially separated from other members of the nucleic acid fragment population, and subsequently from single-stranded nucleic acid template 202.

Depending on the nature of the single-stranded template(s), fragment populations isolated in this way can correspond to either the sense or antisense orientation of the structural genes of interest. Furthermore, capturing complementary populations of interest using opposite strand templates provides a useful population of fragments for mixing with the first (e.g., opposite strand-captured) population for gene reassembly, as described with respect to downstream recombination and the references therein.

As discussed in greater detail below, the nucleic acid fragments isolated according to the methods of the present invention are optionally subject to various downstream processing steps. For example, the isolated fragments can be amplified and/or recombined using a range of techniques including, e.g., polymerase chain reaction, ligase chain reaction, reiterative nucleic acid recombination, single-strand nucleic acid template-mediated recombination, any method herein, or the like. The nucleic acid fragments can be recombined, e.g., to form one or more chimeric nucleic acid sequences or genes, which can be expressed (e.g., in vitro) and the resulting expression product(s) can be screened or selected for a desired trait or property. Chimeric nucleic acid sequences can also optionally be introduced into a host cell prior to expression and selection.

Single-Stranded Nucleic Acid Template-Mediated Recombination

The present invention also provides methods of recombining a set of nucleic acid fragments that can be mediated by a single-stranded nucleic acid template. If sufficient homology exists between the nucleic acid fragments and the template strand, recombination can be accomplished using, e.g., a ligase (e.g., polymerase-free single-strand-mediated recombination). Fragments and template strands lacking sufficient homology for ligase-mediated methods can be recombined by using a polymerase (e.g., a strand-displacing polymerase or a strand-nondisplacing polymerase) and a ligase, e.g., in combination. The polymerase and ligase can each independently be provided either in vitro or in vivo. Each method step can optionally be performed sequentially in a single reaction vessel, or steps can alternatively be performed in separate reaction vessels.

The assembly reaction optionally includes a strand non-displacing DNA polymerase, a thermostable polymerase, a polymerase that includes an intrinsic exonuclease activity, or the like. Many polymerases, both natural and engineered, are known. Suitable DNA polymerases include, e.g., DNA polymerase I (Kornberg or Klenow polymerase), T4 DNA polymerase, T7 DNA polymerase, Taq DNA polymerase, Micrococcal DNA polymerase, alpha DNA polymerase, AMV reverse transcriptase, M-MuLV reverse transcriptase, etc. Suitable RNA polymerases for use in the methods herein include, e.g., an E. coli RNA polymerase, an SP6 RNA polymerase, a T3 RNA polymerase, a T7 RNA polymerase, and an RNA polymerase II. Other known polymerases are available and can be used in the methods described herein.

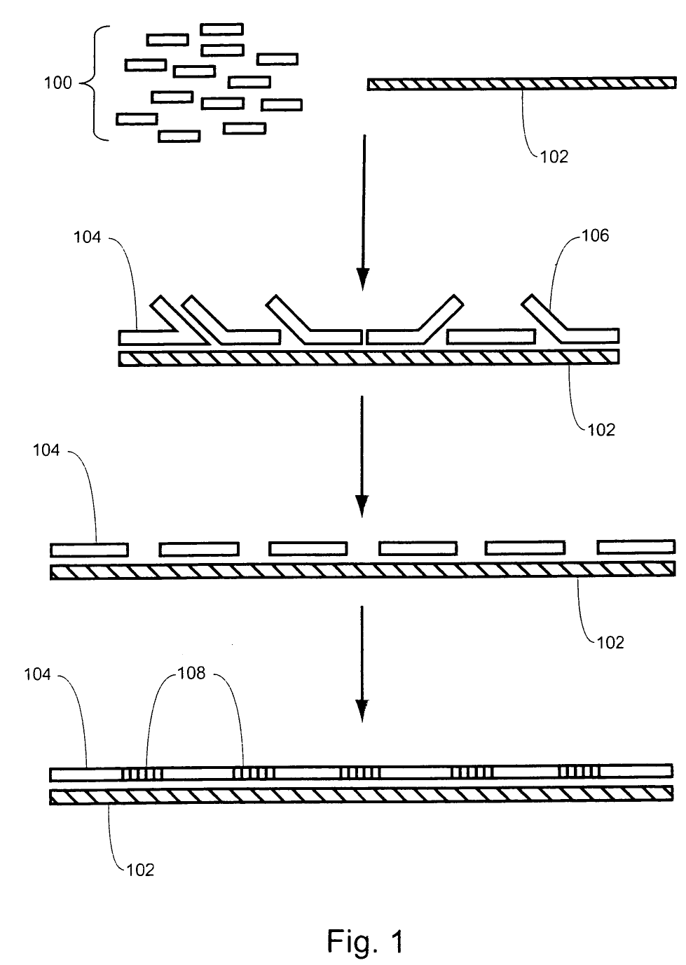

As shown in FIG. 1, one embodiment of single-strand-mediated recombination can include hybridizing at least two sets of nucleic acids, e.g., a first set of nucleic acids including single-stranded nucleic acid template 102 and a second set of nucleic acids that includes nucleic acid fragments 100. Optionally, the methods include cleaving one or more unhybridized portions 106 of hybridized nucleic acid fragments 104, e.g., by nuclease cleavage. The methods can also include separating hybridized nucleic acids 104 from unhybridized nucleic acids by a separation technique, e.g., before or after performing the optional cleaving step. Suitable separation techniques can include, e.g., affinity-based separations, a centrifugation, fluorescence-based separations (e.g., fluorescence-activated particle sorting), magnetic field-based separations, electrophoretic separations, microfluidic molecular separations, chromatographic separations, and the like. As mentioned, depending on the level of homology between the fragments and the template strand, the methods can include elongating and/or ligating sequence gaps 108 between hybridized nucleic acid fragments 104 to generate chimeric nucleic acid sequences that are complementary to single-stranded nucleic acid template 102.

The methods can further include denaturing the chimeric nucleic acid sequences and single-stranded nucleic acid template 102, which can optionally be followed by separating the chimeric nucleic acid sequences from single-stranded nucleic acid template 102 by a separation technique (described above). Thereafter, the separated chimeric nucleic acid sequences can optionally be fragmented by, e.g., nuclease digestion or physical fragmentation to provide chimeric nucleic acid sequence fragments. These chimeric nucleic acid sequence fragments can alternatively be subjected to additional downstream processing steps which are described in greater detail below.

In one embodiment, single-stranded templates are optionally selectively removed, e.g., following nucleic acid fragment reassembly by any of a variety of other techniques known in the art. For example, single-stranded nucleic acid templates are optionally synthesized, either in vitro or in vivo, with the incorporation of uracil into the DNA template, e.g., via PCR with dUTP, or via an E. coli dut⁻ ung⁻ strain (see, e.g., Kunkel et al., (1987) Methods in Enzymology 154:367-381). The degree of uracil incorporation can be controlled. After nucleic acid fragment assembly, as described above, uracil-substituted single-stranded templates are optionally fragmented with two enzymes: Uracil N-Glycosylase (Ung) which hydrolyzes the n-glycosidic bond between the deoxyribose sugar and uracil to generate apurinic (or AP) sites, followed by the use of a 5′ AP endonuclease, such as Endonuclease IV (End) which cleaves a single strand of DNA 5′ to AP sites, leaving a 3′-hydroxy-nucleotide and 5′-deoxyribose phosphate termini. See, e.g., Freidberg et al. (1995) DNA Repair and Mutagenesis, pp. 1-698, ASM Press, Washington, D.C. As used herein, the term “Ung-End fragmentation” refers to uracil N-glycosylase-5′ AP endonuclease-mediated fragmentation. Template fragment size upon Ung-End fragmentation is a function of uracil content which is readily controlled in PCR.

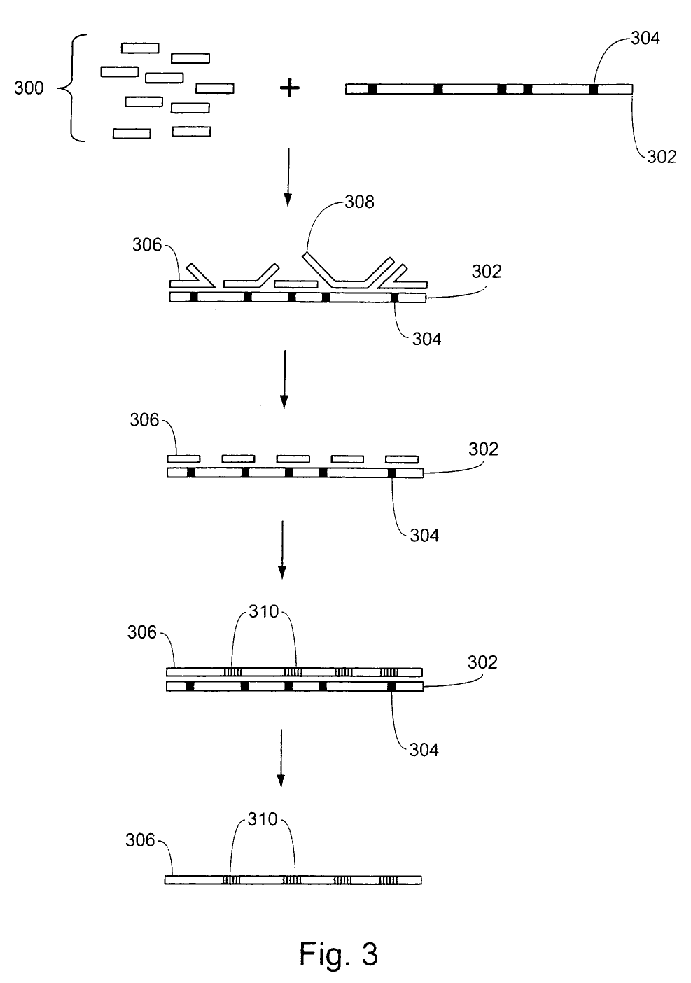

FIG. 3 illustrates Ung-End template fragmentation. As shown, at least two sets of nucleic acids are optionally hybridized, such as a first set that includes uracil-substituted single-stranded nucleic acid template 302 and a second set that includes nucleic acid fragments 300. Uracil-substituted single-stranded nucleic acid template 302 includes one or more deoxy-uracils 304 in place of thymidine(s). Optionally, the methods include cleaving one or more unhybridized portions 308 of hybridized nucleic acid fragments 306, e.g., by nuclease cleavage. The methods can also include separating hybridized nucleic acids 306 from unhybridized nucleic acids by a separation technique, e.g., before or after performing the optional cleaving step. As above, suitable separation techniques can include, e.g., affinity-based separations, a centrifugation, fluorescence-based separations (e.g., fluorescence-activated particle sorting), magnetic field-based separations, electrophoretic separations, microfluidic molecular separations, chromatographic separations, and the like. Furthermore, depending on the level of homology between the fragments and the template strand, the methods can include elongating and/or ligating sequence gaps 310 between hybridized nucleic acid fragments 306 (either in vitro or in vivo) to generate chimeric nucleic acid sequences that are complementary to uracil-substituted single-stranded nucleic acid template 302.

The methods optionally further include denaturing the chimeric nucleic acid sequences and uracil-substituted single-stranded nucleic acid template 302, prior to Ung-End fragmentation of the uracil-substituted single-stranded nucleic acid template 302, as described above. Intact chimeric nucleic acid sequences are optionally separated from the resulting uracil-substituted template fragments by separation techniques, such as those mentioned above (chromatography, electrophoresis, chromatography, etc.). Thereafter, the chimeric nucleic acid sequences are optionally subjected to additional downstream processing steps which are described in greater detail below.

Uracil glycosylases and 5′ AP endonucleases are ubiquitous. They have been characterized in both eukaryotic and prokaryotic cells, as well as viruses (Freidberg et al. (1995)), supra. Many of these can be used for Ung-End fragmentation.

In addition to cleaving 5′ to AP sites, AP nucleases (such as Exonuclease III, Endonuclease IV, and Endonuclease V) recognize and cleave DNA at sites damaged by oxidizing agents or alkylating agents. Endonuclease V additionally cleaves DNA at A/C and A/A mismatches and at deoxyinosine. Thus, the use of controlled dITP (or other non-adenine, non-cytosine, non-guanine, or non-thymine bases) incorporation (e.g., during oligonucleotide synthesis of the single-stranded templates of interest) and Endonuclease V treatment enables a single enzyme method for DNA fragmentation.

Single-stranded nucleic acid templates are also rendered selectively removable using other well-known techniques. For example, templates are optionally synthesized to include RNA single-stranded templates which are selectively digestible (e.g., in the presence of reassembled chimeric DNA fragments), using various well-characterized RNAses. See e.g., Shen, V. and Schlessinger, D. (1982) The Enzymes XV (Part B) 501, delCardayre, S. B. and Raines, R. T. (1995) Anal. Biochem. 225, 176, Johnson, M. G. (1996) Epicentre Forum 3(4), 7, Meador, J. et al. (1990) Eur J. Biochem. 187:549; and Meador, J and Kennell, D. (1990) Gene 95:1. Conversely, single-stranded template strands are optionally synthesized to include DNA for use in RNA fragment recombination. The single-stranded DNA template is selectively digestible in the presence of chimeric RNA sequences using a variety of known DNAses, exonucleases, endonucleases, or the like. Many RNAses, DNAses and other suitable enzymes are readily available from various commercial sources including, e.g., Promega Biosciences, Inc. (www.Promega.com), Epicentre Technologies Corp. (www.epicentre.com), or the like. Other options include selectively digesting the template strand using Exonuclease III (i.e., when the chimeric/template includes a recessed or blunt 3′ end) or any other nuclease which selectively degrades one strand of a duplex, e.g., according to whether the duplex comprises a blunt 5′ or 3′ end, or whether 5′ or 3′ end of the template strand overhangs or is recessed relative to the chimeric strand.

Any of the techniques discussed above are optionally used to digest template strands, while leaving assembled chimeric nucleic acid strands intact. The chimeric strands can then be used as substrates for various downstream processing steps including, e.g., as templates for the synthesis of a second strand that is complementary to the template.

Another embodiment of these methods is schematically illustrated in the sequence of steps that conclude on the right-hand side of FIG. 2. As shown, the methods can include hybridizing at least two sets of nucleic acids, e.g., a first set of nucleic acids can include single-stranded nucleic acid template 202 which can optionally include affinity label 204 (e.g., biotin, digoxigenin, digoxin, a hybridization “tag” or “tail” or the like) and a second set of nucleic acids that includes nucleic acid fragments 200. As mentioned, depending on the level of homology between single-stranded nucleic acid template 202 and nucleic acid fragments 200, the entire length of some fragments can substantially hybridize, while other hybridized fragments can include one or more unhybridized portions 206. As shown, fragments lacking complementarity to single-stranded nucleic acid template 202 remain unbound.

The methods can also optionally include separating the hybridized nucleic acids from unhybridized nucleic acids by various separation techniques (mentioned above). As shown in FIG. 2, a preferred separation method includes binding a detector or capture complex that includes binding agent 208 linked to magnetic bead 210. As mentioned above, suitable binding agents (e.g., avidin, streptavidin, anti-digoxigenin, or the like) linked to magnetic beads are readily available from various commercial sources. Single-stranded nucleic acid template 202 with hybridized nucleic acid fragments 200 can be, e.g., captured by applying magnetic field 212 which acts on magnetic bead 210. Upon capture, unhybridized fragments can, e.g., be washed away leaving the captured hybridized complexes. As a further option, either before or after separating hybridized from unhybridized fragments, one or more unhybridized portions 206 can be cleaved by nuclease digestion (e.g., an exonuclease). Optionally, hybridized nucleic acid fragments 200 can be recombined using, e.g., a polymerase and/or a ligase prior to being separated from unhybridized fragments. However, as depicted in FIG. 2, cleavage and separation can also be followed by elongation and/or ligation to fill in sequence gaps 214 between hybridized nucleic acid fragments 200 to generate chimeric nucleic acid sequences that complement single-stranded nucleic acid template 202.

Following recombination, the resulting chimeric nucleic acid sequences are optionally separated from single-stranded nucleic acid template 202 by denaturation (e.g., by applying heat, etc.) while maintaining the capture of single-stranded nucleic acid template 202 in magnetic field 212. Other separation techniques, such as those mentioned above can also be used.

The resulting chimeric nucleic acid sequences produced by the methods described herein can optionally be used as substrates for various downstream processing steps. For example, the chimeric sequences can be amplified by PCR or a comparable technique, and the amplified chimeric nucleic acid sequences can, e.g., be selected for a desired trait or property of an encoded expression product, e.g., following in vitro or in vivo expression. Alternatively, the chimeric nucleic acid sequences can be introduced directly into a suitable host cell (e.g., a host cell tolerant to mismatches) and be expressed to provide an expression product to the cell (e.g., an E. coli mutS strain). A further option can include fragmenting the amplified chimeric nucleic acid sequences by nuclease digestion (e.g., DNAse, RNAse, endonuclease, exonuclease, and the like) or by physical fragmentation to provide chimeric nucleic acid sequence fragments. The chimeric nucleic acid sequence fragments can subsequently be used, e.g., as substrates for further recombination (e.g., additional single-stranded nucleic acid template-mediated recombination, reiterative nucleic acid recombination, or the like), as substrates for the methods of isolating a set of nucleic acids fragments (described above), and the like. A wide variety of upstream and downstream processing techniques are described herein; these techniques, as well as other available techniques can be used to modify any chimeric sequence produced by any method herein.

Nucleic acid templates employed in the practice of the present invention are optionally either substantially all sense strand templates or substantially all antisense templates. Suitable nucleic acid fragments include either double-stranded or single stranded fragments (double-stranded fragments can also be converted to single-stranded fragments, and vice-versa, e.g., using standard hybridization methods). Single-stranded fragments can be from packaged phagemid DNA or generated according to any one of the methods described herein (denaturation of double-stranded sequences, oligonucleotide synthesis, etc.). If single-stranded fragments are used, the set of nucleic acid fragments can be either substantially all sense strand fragments or antisense strand fragments. For example, a set of substantially all sense strand templates can be used together with a set of substantially all antisense strand fragments, or vice-versa.

Nucleic acid fragments that are suitable for use in the practice of the present invention generally include those that are from about 5 bp to about 5 kbp is size, although larger size can also optionally be used. Typically, nucleic acid fragment size is from about 10 bp to about 1000 bp, more typically the size of the fragments is from about 20 bp to about 500 bp. The number of different nucleic acid species (i.e., with respect to both size and sequence) in the set of nucleic acid fragments is e.g., at least about 5, e.g., typically at least about 10, or typically more than about 20 or more.

The optimal ratio of fragments to templates employed can vary depending on the size of fragments and templates employed. One of ordinary skill in the art can readily determine the optimal ratio by varying this ratio with respect to the particular set of template nucleic acids used, as illustrated, e.g., in Example 11, below. At the lower range of fragment:template weight ratios, typically, the fragment:template ratio is at least about 0.2:1, more typically at least about 0.5:1, and usually at least about 1:1 or 2:1. An excess amount of fragments can be used, for example, fragment:template (e.g., weight to weight) ratios of at least about 10:1, at least about 50:1, at least about 100:1, at least about 250:1, at least about 500:1, at least about 1,000:1, at least about 1,500:1, or at least 10,000:1 or more are all suitable depending on the fragment and template size used, and the results desired.

After hybridization, the polymerization, ligation, and optional cleaving steps can be carried out in vitro, in vivo, or a combination of both in vitro and in vivo. If some or all of the steps are carried out in vivo, the hybridized complex is transformed into a host, e.g., that is defective in mismatch repair, e.g., an E. coli mutS strain. The host cell thus provides the enzymes (e.g., polymerases, ligases, and exonucleases) required to generate a complete duplex.

Alternatively, the chimeric strand/template duplex can be denatured, followed by PCR amplification, transformation and screening. In a further alternative embodiment, the template can be degraded, a complementary strand synthesized, followed by amplification, transformation, and screening of an expression product of the chimeric strand or one complementary thereto.

For in vitro recombination, suitable polymerases employed in the invention method include both strand-displacing (e.g., Pfu, Klenow, and the like) and non-strand-displacing polymerases (e.g., a T4 DNA polymerase, a T7 DNA polymerase, T7 Sequenase DNA polymerase, Taq, Stoffel fragment of Taq, E. coli Pol I, and the like). Preferably, the polymerase is a mesophilic polymerase (i.e., active at temperatures at about 45° C. or less, typically active at temperatures of about 40° C. or less, more typically, active at temperatures between about 40° C. or less, more typically, active at temperatures between about 40° C. or less, e.g., 37° C. or less, e.g., about 25° C. or less e.g., about 16° C. or more)), e.g., a T4 DNA polymerase, a T7 DNA polymerase, T7 Sequenase DNA polymerase, E. coli Pol I, and the like. Preferably, the polymerase is both non-strand-displacing and mesophilic. Ligases contemplated for use in the practice of the present invention include, e.g., T4 RNA ligases, T4 DNA ligases, E. coli DNA ligases, or the like. A nuclease, or a polymerase with nuclease activity (e.g., Pol I), can be used, e.g., to cleave the unhybridized portions of partially hybridized fragments. Many nucleases suitable for use in the methods described herein are well-known in the art.

When carrying out all or part of the recombination reaction in vitro, the mixture of hybridized templates and fragments are incubated with appropriate enzymes to carry out a desired reaction. For example, if recombination reactions are carried out in vitro, mixtures of hybridized templates and fragments can be incubated with a polymerase, a ligase, and, optionally a nuclease such as an exonuclease, in a single vessel. Alternatively, as described above, part of the reaction, e.g., polymerization, can be carried out in vitro (in which case only the polymerase is incubated with the mixture), and the ligation reaction can be carried out in vivo.

Typically, the incubation temperature is between about 4° C. and about 75° C., and more typically, 45° C. or less, e.g., 40° C. or less, e.g., 37° C. or less, e.g., about 25° C. or less e.g., about 16° C. or more or less, or about 4° C. or more). Prior to incubating with one or more of the recombination enzymes, the mixture can be heated to about 95° C. or more, then slowly cooled to allow the fragments to anneal to the templates. This step helps among other things, to minimize formation of secondary and tertiary nucleic acid complexes between single stranded DNA, and if double stranded fragments are used, to denature the fragments.

To illustrate, nucleic acid fragments from coding strand derivatives can be mixed with antisense strand templates (e.g., phagemid templates). The fragment-template mixture is heated to about 95° C. for about 3 minutes, then gradually cooled to room temperature to allow the single stranded fragments to anneal to the single strand templates. Thereafter, dNTPs, a polymerase, and a ligase are added to the mixture and incubated for about 2 hours at, e.g., 37° C., to extend and ligate the fragments over the template to generate chimeric nucleic acid molecules. The resulting chimeric nucleic acids can be transformed into, e.g., an E. coli mutS strain that is defective in mismatch repair to enrich for chimeric clones.

The single-stranded template-mediated recombination methods of the invention include many other alternative parameters that can be selected to optimize, or otherwise customize, the particular recombination reactions being contemplated. For example, the methods optionally include the use of a non-strand displacing polymerase (e.g., a T4 DNA polymerase or the like) to extend fragments over the template. A lack of strand-displacement activity can facilitate chimeragenesis (production of chimeric nucleic acids) by, e.g., permitting ligation to occur following extension of adjacent fragments over the template. As described further below, extensions catalyzed by non-strand displacing polymerases are also optionally used to generate single- or double-stranded nucleic acid fragment populations. Alternatively, strand-displacing polymerases, such as the Klenow polymerase or the like are optionally used. Note, that highly processive enzymes, such as Klenow polymerases, are also optionally used in, e.g., certain methods of preparing single-stranded nucleic acid templates, which are described below.

The present invention also includes methods of assembling recombined partial genomes using single-stranded fragments and phagemid templates. For example, fragments from coding strand derivatives can be mixed with antisense strand template at, e.g., fragment-template molar ratios of about 5, 10, 50, 100, 250, or more. Fragment-template mixtures are then typically heated to about 95° C. for 3 minutes and gradually cooled to room temperature to allow the single strand fragments to anneal to the single strand templates. Thereafter, dNTPs, a polymerase (e.g., a T4 DNA polymerase or the like), and a ligase (e.g., a T4 DNA ligase or the like) are added mixture and incubated for about 2 hours at, e.g., 37° C. to extend and ligate the fragments over the template to generate chimeric nucleic acid molecules. The resulting chimeric nucleic acids are optionally transformed into a suitable expression host. Preferred hosts include, e.g., an E. coli mutS strain that is defective in mismatch repair to enrich for chimeric clones. Transformed hosts are then typically selected for one or more desired traits or properties as described herein.

In one illustrative embodiment, partial genomic fragments are cloned into F′-derived phagemid vectors (‘fosmids’) which have the ability to incorporate and transfer large fragments of DNA between microbial hosts. Such fragments generally exceed 10 kb in length and are, e.g., more than 25 kb in length. Cells carrying such fosmids or fosmid libraries are used as donors to transfer the partial genome fragments (in single stranded form) to a recipient cell line. Recipient cells lacking the biological, synthetic or chemical property believed to be encoded by the fragmented genome are then screened for development of this and/or other properties following a transduction or conjugation step in which some or all of the fosmid DNA is transferred to the recipient cells.

As noted throughout, the methods of the present invention can be practiced in a single cycle of recombination (e.g., template-based recombination) or can be practiced in a recursive fashion with more than one cycle of recombination being performed. Activity selection steps can be performed after one or more recombination step (i.e., after single or multiple rounds of recombination) to provide new or improved activities or other properties of interest. Furthermore, repeated cycles of recursive recombination/selection can be performed recursively to provide further improvements sought in any activity or other property of interest, or to provide new properties of interest.

Additional Details on Single Stranded Template-Mediated Recombination Approaches

A variety of single-stranded template-mediated recombination techniques are included in the present invention and are set forth herein. These include, e.g., in vivo or in vitro recombination, or combinations thereof, combinatorial nucleic acid sequence assembly and/or mutagenesis, template-based assembly of synthetic and mutagenized gene libraries, use of bridging oligonucleotides for single-stranded chimeric fragment production/isolation, construction of single stranded combinatorial mutagenic cassettes via direct synthesis of a multiplexed single mutant oligonucleotide array, site-specific restriction digestion of single stranded template DNA, forced recombination between folding domains or domain segments using bridging oligonucleotides and a variety of other methods that will become apparent upon complete review of the foregoing and following.

In one aspect, single-stranded templates are, e.g., all or part of a gene used to isolate, construct, fine tune, generate, amplify or otherwise “capture” recombination cassettes/chimeric nucleic acids, or substrates from characterized or uncharacterized nucleic acid populations samples (e.g., synthetic nucleic populations, library or plasmid DNA samples, or the like). In each case, the template is optionally eliminated or modified, either biologically (in vivo), or via an in vitro selection enzyme (e.g., a methylation sensitive restriction endonuclease, a specific or non-specific endo- or exonuclease, or the like) or via physical separation or capture, e.g., via one of many available magnetic, affinity or ‘panning’-based separation procedures, or by any other available method(s). In many cases, physical separation methods utilize elevated temperatures (e.g., a temperature higher than the melting temperature, i.e., T>Tₘ) or chemical denaturants and subsequent cooling (or extraction). “Templated cassettes” prepared in this way can be used to prime nucleic acid extension or recombination reactions. Second strand synthesis can be directed by short end overlap primers, random primers or by annealing to a complementary synthetic nucleic acid populations at high stringency. Partially overlapping cassettes can be reassembled by high stringency primeness extension PCR (e.g., run at annealing temperatures of T>Tm-10° C.). Another alternative is the defined recombination of fixed recombination regions of 1-100 bases which remain fixed and drive the ordered assembly of synthetic genes. These and other alternatives are discussed herein.

Combinatorial Nucleic Acid Sequence Assembly/Mutagenesis

As noted, in one aspect, the present invention includes methods for combinatorial nucleic acid sequence assembly and/or mutagenesis, including non-enzymatic recombination methods. One embodiment of the methods of the invention includes, e.g., providing a first population of single stranded template polynucleotides which hybridize to a second population of polynucleotide fragments which the hybridization directs combinatorial assembly of a third polynucleotide population based on the hybridization of the first and second populations. The methods also typically include selecting or screening the assembled third polynucleotide population for expression products having one or more desired traits or properties. These combinatorial assembly methods can be performed in vitro or in vivo, via enzymatic or non-enzymatic recombination mechanisms.

For example, as already noted, the methods of the invention can include assembly of the second population of nucleic acids using a first population of templates, e.g., via hybridization of the first and second population, followed by ligation, elongation, digestion of unhybridized segments, etc. Typically, more than one and often 5, 10, 20, or more fragments from the second population will hybridize to a template. A third population of nucleic acids is produced following elimination of the templates via any of the many approaches noted herein, or any others that are available, optionally followed by second strand synthesis.

In a related alternate embodiment, a partially enzymatic or a non-enzymatic recombination approach is used. In this approach, the first population is used as a template for assembly of the second population of nucleic acids, e.g., via hybridization. The hybridized complex can then be transduced into a cell, where the cellular nucleic acid repair machinery (generally DNA repair machinery) treats the hybridized nucleic acids as polymerase primers, ligation sites, mismatch sites etc. for mismatch repair, elongation of nucleic acids via polymerase mediated mechanisms, exonuclease digestion of unhybridized regions, ligation of adjacent nucleic acids, etc. Thus, the non-enzymatic approaches actually involve the use of enzymes, but the enzymes are provided by the cell, rather than directly by the user in an in vitro system. Put another way, the cell is used to perform any reaction that can be performed in vitro. In one aspect, the first and second sets of nucleic acids including overlapping members, which can, e.g., facilitate cellular repair.

At least some of the differences between templates and hybridized nucleic acids are present in nucleic acids which result from action of the cellular machinery on the nucleic acids; thus, the procedures produce chimeric nucleic acids which can be selected or screened as noted herein.

In some approaches, nucleic acids are further diversified by transducing the hybridized nucleic acids into mutable or hyper-mutable cell strains, e.g., those that are deficient or overactive in one or more repair or recombination enzyme. A variety of such cell types are known, including those with alterations in muts, mutL, and a variety of other repair systems. A variety of such systems are noted in the references incorporated herein. Similarly, cells that are engineered to constitutively or inducibly overexpress or underexpress any enzyme relevant to the process of recombination can be used in the methods herein. In both the in vitro and in vivo embodiments herein, mutant forms of these enzymes (e.g., polymerases, nucleases, ligases, etc.) can be used where the properties of the mutant enzymes is useful to the procedure at issue.

While the above was described in terms of the use of a cell to provide nucleic acid modification systems, it is worth noting that cellular extracts can also be used, e.g., any cellular extract that has any of the activities relevant to the methods noted herein.

In other aspects, partially in vitro enzymatic/partially in vivo approaches to recombination are used. That is, any of the relevant enzymatic treatments (ligase, polymerase, nuclease, etc.) can be performed prior to transfer of the resulting nucleic acids into one or more cells, where the cellular machinery performs further modification of the nucleic acids.

In one aspect, and as noted in more detail herein, hybridized nucleic acids can be nicked with one or more nucleases (e.g., Mung bean nuclease) or chemically modified, to produce sequence gaps or other lesions, which can be repaired by the cellular machinery. This approach can be used to increase the diversity of chimeric nucleic acids that result after repair by the cell or other in vivo system (or that result from similar repair in an vitro system).

In any case, combinatorial assembly optionally uses any of the nucleic acid ligases noted herein, e.g., where the nucleic acid ligase exhibits a gap repair activity. Optionally, the nucleic acid ligase is present in an in vitro reaction mixture. Alternatively, as noted, the nucleic acid ligase can be supplied by host cells transformed with one or more members of the third polynucleotide population. Similarly, the assembly of the polynucleotide fragments from the second population also optionally includes a DNA or RNA polymerase, including any of those noted above and any that may exist in a cell transduced with a nucleic acid of the invention. As noted above, the methods for combinatorial nucleic acid sequence assembly can also include the use of a nuclease, including any of those noted above.

While it should be apparent from the foregoing, it is noted that the assembly methods herein optionally include the use of various combinations of enzymes, such as a polymerase and a ligase; a ligase and a nuclease; a polymerase and a nuclease, a nuclease, a ligase and a polymerase, or any other possible combination, including the use of any of these combinations with in vivo cellular systems that are accessed by transducing a cell with one or more nucleic acid of interest, or cellular extracts that are incubated with nucleic acids to be recombined. For example, in one typical embodiment, polymerases are used in vitro to perform primer extension (or primerless PCR or other polymerase extension procedures) on the template, with ligation being performed by the cell. In another typical embodiment, ligase is used in vitro, with polymerase and/or exonuclease functions being performed in vivo. Any other permutation of enzymatic treatment and cell-based repair can also be used.

As will be described in more detail below, proteins or protein fragments derived from the chimeric third polynucleotides which are produced by assembly as noted, are optionally selected for one or more physical properties including, e.g., altered temperature (e.g., in the range of less than about 20° C., or greater than 50° C., or any other desired range, including those noted herein) or pH range or optima (e.g., in a pH range of less than about 5.5 or greater than about 8 or any other desired range, including those noted herein), stability, tolerance to presence of solvent, oxidant, salt, surfactant and/or other solutes, process specific physical environments, or the like. Indeed, any property of interest, including, e.g., any of those noted in more detail herein, can be screened for, using, e.g., any available method, e.g., including those noted herein.

For example, a specific screens of interest includes, e.g., evaluation of enzyme performance in non-aqueous and semi-aqueous systems (e.g., in which the system includes crude oil or distillation fractions derived from crude oil and in which the polynucleotides to be screened are expressed in whole cells). For example, these screens optionally include assessing the rate or extent of substrate desulfurization and/or measuring the appearance or disappearance of organic or inorganic sulfur. Many other suitable assays or screens for use with these methods are discussed herein.

The methods optionally include high-throughput systems such as automated mechanical steps in which one or more polynucleotide samples are moved using a robotic arm, a robotic platform, or other computer-controlled electromechanical devices. In addition, selected or screened polynucleotides (or propagatable forms thereof) are sequenced, or the selecting or screening step is followed by a logical cataloging step. Optionally, the third polynucleotides, their progeny and/or derivatives are screened for an increase or decrease in immunogenicity, allergenicity, or potential hypersensitivity. Alternatively, or in addition, FACS is optionally used to enrich, sort, analyze or otherwise evaluate cells or other particles containing the selected polynucleotides. Assembled polynucleotides or expression products therefrom are organized in arrays (e.g., physical, logical, or the like). For example, the third polynucleotide population is optionally cataloged based on sample origins, screening data, physical location, or other identifying properties. Many details regarding array-based screening and recombination methods, including automated methods, are found in U.S. Ser. No. 60/213,947 by Bass et al., entitled “INTEGRATED SYSTEMS AND METHODS FOR DIVERSITY.”

Template-Mediated Assembly of Synthetic and Mutagenized Gene Libraries

The invention provides, e.g., methods of assembling synthetic and mutagenized gene libraries that are mediated by single-stranded templates. Note, that although the following discussion occasionally refers to the subtilisin E amino acid and nucleic acid sequences for purposes of illustration, it will be appreciated that any parental sequence of interest (including, e.g., natural, or artificial sequences, including naturally occurring or recombinant or mutant sequences) is optionally used in these methods. Many single-stranded nucleic acid template and nucleic acid fragment sources are described herein.

This method generally includes generating single-stranded DNA templates corresponding to the sense or antisense strand of a parental sequence of interest, such as subtilisin E, or the like, using a phagemid vector. Sense and antisense orientations can be controlled, e.g., by changing the direction/orientation of the origin of replication., so you can make either + or − strands.

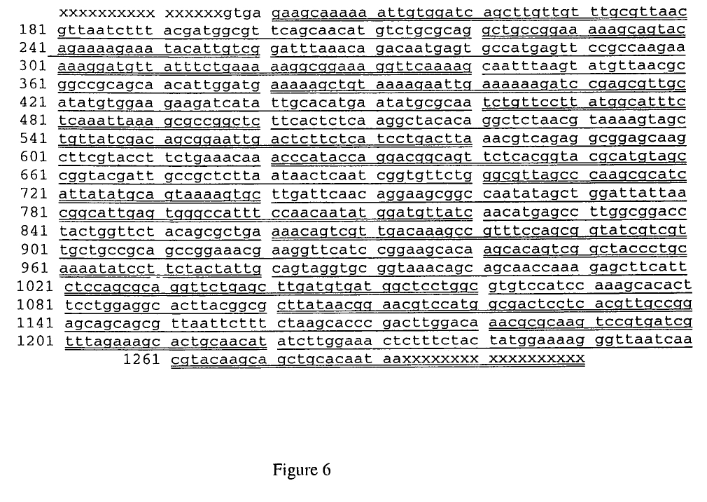

Alternatively, sense or antisense strands of DNA may be generated via other techniques known in the art, including those described above. Additionally, oligonucletotides are synthesized which correspond, e.g., to the subtilisin E amino acid and nucleic acid sequences. For example, the subtilisin E nucleic acid sequence is shown in FIG. 6.

For example, mutagenic 40mer oligonucleotides which correspond to subtilisin E are synthesized to allow approximately (1-1/target length)×100% wild-type sequence at each codon position and (1-1/target length)×100% N,N,(G/C) frequency. This can be accomplished by, e.g., operating an automated oligonucleotide synthesizer (e.g., the PCR-Mate series from Applied Biosystems) such that each coupling cycle, over a targeted region, is conducted so that an appropriate fractional volume of mixed precursors is drawn from a vial containing the wild-type base and a vial containing an appropriate randomizing mixture. For example, the randomizing mixture might include the other three bases, a G/C mixture (e.g., where the wild-type sequence is A or T), or vials containing only G or C (e.g., when the wild-type base is the complement of one of these). Furthermore, these combinatorial cassettes are optimally synthesized with 5′ phosphate groups and 3′OH groups, and end and start on adjacent codons to allow for efficient ligation. To further illustrate, non-overlapping 40 mers which correspond to the sequence of subtilisin E are depicted in FIG. 6. Note, that each alternating double underlined and single underlined region represents a ˜40mer oligonucleotide synthesized in this method with the described level of mutation. Such mutant oligonucleotides may be assembled, for example, by annealing to an excess of single-stranded antisense (e.g., in this case subtilisin) DNA, followed by ligation and separation or degradation of the template strand.

In FIG. 6, x's indicate sequences that optionally do not correspond to wild-type sequences which may be replaced by upstream regulatory regions and vector supplied sequences depending on the cloning system in use. For example, the 3′ and 5′ untranslated regions can correspond identically to those described in, e.g., Zhao and Arnold (1997) “Functional and nonfunctional mutations distinguished by random recombination of homologous genes,” Proc. Natl. Acad. Sci. U.S.A. 94(15):7997-8000 and H. Zhao, et al., “Molecular evolution by staggered extension process (StEP) in vitro recombination,” Nature Biotechnology (March 1998), 16(3):258-61, and thereby be amenable to the expression and screening systems described therein.

To assure development of maximum diversity, primers are optionally annealed under conditions of an excess of the single-stranded template (e.g., 10 pmol per primer: 20 pmol single-stranded template) and at a temperature of less than Tm-10° C. (e.g., in this case about 50° C.). In brief, mixtures containing oligonucleotides and single-stranded template molecules are heated to 99° C. for 2 minutes, then gradually cooled over 2 hours to 16° C. Terminal primers are included in the mixture which overlap with segments just 5′ and 3′ of the region targeted for mutagenesis and which are suitable for facilitating priming and incorporation into vectors or alternative expression constructs. Thereafter, the annealing mixture is adjusted with ligation reaction components, e.g., 5 Units of T4 DNA ligase and ATP. The ligation reaction is allowed to proceed overnight at 13° C.

Template strands are optionally separated or eliminated using methods described herein, or otherwise known in the art. For example, the template strand can be selectively degraded with exonuclease III as described herein. Thereafter, the single stranded mutant population of product is typically amplified, e.g., using flanking primers such as P5N and P3B in the illustrated case of subtilisin E. The resultant double stranded mutant population is then typically ligated into an expression vector and screened as described herein.

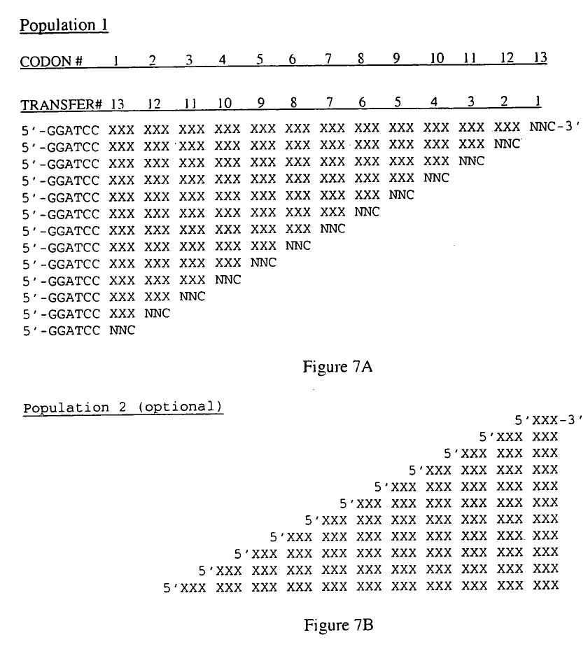

In an alternative embodiment of the methods of assembling synthetic and mutagenized gene libraries that are mediated by single-stranded templates, described above, oligonucleotides are synthesized in such a way as to end in a single redundant codon. For example, this is accomplished by first preparing two batches of resin containing either *N—N-G-resin or *N—N—C-resin (where * indicates the attachment end at which new bases are added during synthesis). This can be accomplished using an automated DNA synthesizer according to methods known in the art. For example, a fixed mass (e.g., 10 mg) of *N—N—C is added to the reaction vessel following each trinucleotide coupling set. All subsequent reaction steps are then shared by the progressively accumulated resin. Fresh resin is added after each trinucleotide synthesis step to allow generation of an oligo with a redundancy at each position. As shown in FIG. 7A, invariant recombination and digestion sites are optionally incorporated within the backbone structure derived from the oligonucleotide sequences. As an alternative to the single base coupling cycle described above, vials containing preformed trinucleotides encoding the amino acid or set of amino acids desired at a given position are optionally included. As shown in FIG. 7A, the transfer # indicates the trinucleotide synthesis step at which the progenitor resin is added in order to give the listed sequence. For example, each transfer is optionally transferred to a single synthesis vessel in which the same base is added to each oligonucleotide at each reaction cycle after the redundant codon is incorporated.

Optionally, a second population of staggered, non-redundant oligonucleotides can be synthesized which fill in the space left open due to the termination of the oligo at the redundant codon. This population is generated in an analogous manner, as above, except that removal of a given aliquot of resin is not followed by performance of additional synthesis steps on the removed strand. To optimize hybridization properties it is ideal if the second population extends at least 6 bases beyond the 3′ terminus of the Population 1 sequences. The simplest filler population for the family described above is depicted in FIG. 7B. Note, that X's are used to indicate that the synthesis of a defined codon in each of these positions, most typically correspond to template or wild-type sequences, or a very limited variation of these. (FIG. 7B).

It will be appreciated that the redundant codon can form either the extreme 5′ position of a set of oligonucleotides or the extreme 3′ end. Furthermore, the NNC containing population can optionally be added back to the main synthesis vessel to syntheize oligonucleotides with multiple mutations if that is desired. In addition, any one, two or three nucleotides in a codon may be varied according to this approach.

To establish the mutant single-stranded recombination cassette, populations 1 and 2 (see FIGS. 7A and 7B) are added in substantial molar excess (>1.5:1) to a mixture containing single stranded template (1 μg) corresponding to the opposite strand. The solution (e.g., 1× ligation buffer minus ATP) is heated to 99° C. for 2 minutes, then cooled over 20 minutes to room temperature. ATP and T4 ligase are added to the mixture and the solution is incubated overnight at 13° C.

A pool of assembled mutagenic strands is typically isolated by, e.g., denaturation and preparative gel electrophoresis. A similar process is followed for each set of mutagenic oligonucleotides until each region is covered by a mutagenic cassette. For complete gene recombination and reassembly of singly mutant genes, a single mutagenic cassette is annealed to template mutagenic cassette in the presence of defined oligonucleotide sequence such as illustrated in FIG. 6 for the remaining segments of the gene. The single stranded full-length library is assembled by annealing the fragments to a full length gene immobilized on a separable, non-protein binding matrix, followed by addition of ligase, then by denaturation and precipitation of the eluted full length, combinatorially assembled single stranded DNA population. Following single strand isolation, the population is amplified, expressed and screened using any of a wide number of available in vitro and in vivo systems as described herein.

Construction of Single Stranded Combinatorial Mutagenic Cassettes Via Direct Synthesis of a Multiplexed Single Mutant Oligonucleotide Array

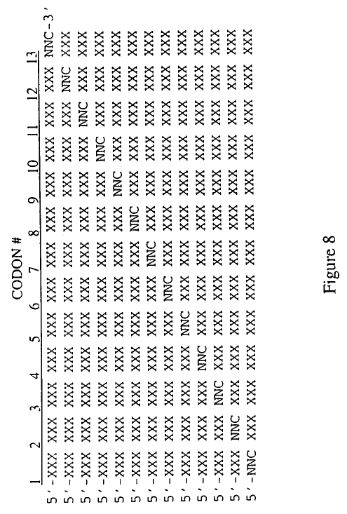

In a more complex synthesis regime, mutant recombination cassettes may be synthesized directly. For example, the oligonucleotides described with respect to FIG. 6 are optionally synthesized mutagenically by synthesizing separately each of the 13 single codon mutagenized (NNC) oligos corresponding to each of the 40mers, excluding the last oligonucleotide which only partly encodes the sequence of interest. Briefly, synthesis is conducted in separately controlled flow cells for each of the desired sequences, resulting in approximately [(28×13)+(1×7)=]91 distinct synthesis reactions, followed by the pooling of those sequences corresponding to common recombination cassettes. See, FIG. 8. For example, oligonucleotides are optionally added in substantial molar excess over template (e.g., >1.5:1) to a mixture containing single stranded template (e.g., about 1 μg) corresponding to the opposite strand. The solution (e.g., 1× ligation buffer minus ATP) is heated to 99° C. for 2 minutes, then cooled over 20 minutes to room temperature. Thereafter, ATP and T4 ligase are added to the mixture and the solution is incubated overnight, e.g., at about 13° C.

While this method allows up to at least one amino acid mutation for each recombination cassette, the level of diversity can be reduced by, e.g., using only a single recombination cassette. The single stranded full-length library is assembled by annealing the fragments to a full-length gene, e.g., immobilized on a separable, non-protein binding matrix, followed by addition of ligase, then by denaturation and precipitation of the eluted full-length, combinatorially assembled single stranded DNA population. Following single strand isolation, the population is amplified, expressed and screened using any of a wide number of available in vitro and in vivo assay systems as described herein.

Site-Specific Restriction Digestion of Single Stranded Template DNA

The invention includes methods for preparing single stranded phagemid DNA capable of annealing to and priming in vitro amplication of the mutagenized and/or synthetically recombined population. The methods include preparing single stranded circular phagemid DNA using the methods described herein and elsewhere in the art. Oligonucleotide primers are typically generated which anneal to the single stranded template in the region overlapping the recombined population. Following annealing of the synthetic oligonucleotides to the single stranded template DNA, the DNA is typically digested in the double stranded region using, e.g., site-specific restriction endonucleases. The resulting sequences are ideal vector primers for capturing and amplifying the libraries described above. For example, equal concentrations of digested single stranded template and cassette recombined populations are mixed and subjected to primerless PCR, purified, transformed into a suitable host (e.g., E. coli or the like), and antibiotic resistant clones are isolated and screened for a desired activity. This method represents one of several ways of conducting ligation-free cloning and expression of recombined or mutant genes. As noted above, a variety of enzymatic steps can be replaced by transducing genes of interest into cells, which perform similar operations in vivo.

Bridging Olilgonucleotides for Single-Stranded Fragment Isolation

Another option includes performing the methods of template-mediated assembly of synthetic and mutagenized gene libraries, described above, except that 15-25mer oligonucleotides extending over overlap regions replace the single-stranded template DNA. The bridging oligonucleotide are optionally redundant (i.e., more than one bridging oligonucleotide) or singular (i.e., one bridging oligonucleotide). Following ligation and/or extension of the opposite strand, bridging oligonucleotides are removed by, e.g., denaturing gel electrophoresis, heat denaturation followed by purification over a sizing column, or other similar methods known in the art for separating oligonucleotide from higher molecular weight DNA. Additionally, while second strand synthesis is optionally conducted by conventional DNA amplification, digestion of single stranded phagemid or single stranded plasmid DNA to which the flanking oligonucleotides in the gene construction have been made complementary can also be used.

Forced Recombination between Folding Domains or Domain Segments Using Bridging Oligonucleotides

The present invention includes designing bridging oligonucleotides to force recombination between, e.g., identifiable folding domains or domain segments, such as between helices and loops, loops and beta sheets, or between strands of a given beta sheet. For example, alph-beta barrel proteins are optionally recombined by aligning members of at least two alpha-beta barrel proteins from at least two subclasses of enzymes. For example, Xanthobacter haloalkane dehalogenase can be recombined with, e.g., at least one other gene encoding an epoxide hydrolase, a carboxypeptidase, an acetyl cholinesterase, a lactone hydrolase, a diene lactone hydrolase, a haloacid dehalogenase, a Renilla luciferinase-like monooxygenase, or the like. Members of any or all of these classes of alpha-beta barrel proteins can be aligned with the Xanthobacter haloalkane dehalogenase whose primary, secondary and tertiary structures are well known and available on the Entrez and other databases. The homologs can be aligned in such a way as to optimize homology in the defined folding regions and a plurality of oligonucleotides can be designed to facilitate gene recombination to occur across these folding elements or sub-elements. For example, any method of gene recombination can be used in the presence of a molar excess of one or more such oligonucleotides. The resulting library can be screened for dehalogenase or other alpha beta hydrolase activities by methods described herein. Clones expressing altered or elevated activities can be selected for further rounds of conventional or forced recombination and rescreened until the desired property is obtained. A further option includes using RNA templates, removing the template by RNase treatment, followed by, e.g., precipitation of ligated single-stranded DNA.

Generation of Chimeric Genes and Gene Pathways by Heteroduplex Repair

In addition to the methods noted above, the present invention includes methods of creating chimeric nucleic acids, e.g., genes or gene pathways, via heteroduplex repair that can optionally be used as additional upstream and/or downstream methods to the other methods noted herein. That is, this method can be used to produce templates or fragments for the other methods noted herein, or to further modify chimeric nucleic acids produced by any other method herein.

This heteroduplex repair method, which can be practiced separately from or in conjunction with the other methods of the invention, can be readily carried out at ambient (e.g., room temperature), as well as higher and lower temperatures. This method, when employed under ambient and lower temperature conditions, is particularly suitable for generating chimeric genes and pathways from low homology “parental” nucleic acid sequences, that would not otherwise hybridize together at higher temperatures.

In accordance with the present invention, chimeric nucleic acids are prepared by hybridizing a first plurality of first parental single-stranded nucleic acids and a second plurality of second parental single-stranded nucleic acids to form a heteroduplex, where the hybridized complex of first and second parental single-stranded nucleic acids includes at least one nonhybridized region of sequence diversity (i.e., a heteroduplex mismatch region). Following hybridization, at least one strand in the nonhybridized region of sequence diversity is nicked and the nicked strand in the at least one nonhybridized region of sequence diversity is cleaved (e.g., degraded such that nucleotides proximal to the nick are removed) to provide at least one sequence gap between hybridized regions. In preferred embodiments, only one strand in the at least one nonhybridized region of sequence diversity is nicked. The number of mismatch regions that are nicked determines the number of chimeric cross-overs in the progeny. Thereafter, the methods include elongating and/or ligating the sequence ends adjacent to sequence gap between the hybridized regions to generate chimeric progeny nucleic acids. Optionally, the hybridizing, nicking, cleaving, and elongating steps are repeated at least once.

The first and second parental single-stranded nucleic acids may encode one or more substantially full-length proteins, or portions thereof. Parental single-stranded nucleic acids suitable for use in the invention method include all of those described herein, as well as natural (e.g., allelic and species variants) and non-natural variants thereof. Typically, the sequences of the first parental single-stranded nucleic acids and the second parental single-stranded nucleic acids differ in at least two nucleotides

Single strands in the heteroduplex can be nicked at regions of mismatch (i.e., in the at least one nonhybridized region of sequence diversity) using, for example, any of a number of enzymes that are known in the art. Suitable enzymes include hairpin specific nucleases (for example, Mung bean nuclease, nickase, or the like) and uracil N-glycosylase. The latter is employed when at least one of the strands in the heteroduplex has uracil incorporated within its sequence. Nicking frequency can be controlled and readily varied by methods known in the art, such as, for example, varying the amount of enzyme employed, varying the amount of uracil in the uracil-containing sequence if uracil N-glycosylase is used, etc.

Uracil-containing nucleic acid sequences are typically prepared by random or nonrandom incorporation of dUTP into the first or second parental single-stranded nucleic acids during synthesis (i.e., synthesis of the parental single-stranded nucleic acids). During the nicking step, the at least one strand in the at least one nonhybridized region of sequence diversity is nicked at one or more sites of dUTP incorporation with a glycosylase (e.g., a Uracil N-Glycosylase) and an endonuclease (e.g., Endonuclease IV). The use of uracil-substituted nucleic acid sequences is discussed further above.

The nicked strands are then cleaved in at least one nonhybridized region of sequence diversity by incubating them with at least one nuclease (e.g., an Exonuclease VII) to degrade/remove the nucleotides proximal to the nicked non-homologous regions. All or just some of the non-hybridized regions of sequence diversity can be nicked, cleaved, and degraded.

The resulting sequence gaps between hybridized regions are typically filled in by elongating and/or ligating the sequence ends adjacent to the gap using, for example, a polymerase and/or ligase, respectively. Optionally, either or both elongation and ligation steps can be conducted in vivo in a suitable host, where the polymerase and/or ligase is provided by the host. Duplexed nucleic acids containing mismatched regions (i.e., regions that were either not nicked, cleaved, or degraded) can be introduced into a suitable host cell for in vivo repair of intact, mismatched regions as described in WO 99/29902. Thus, products of the invention method, which include, for example, heteroduplexes containing single-stranded sequence gaps and/or nicks, as well as mismatch regions, and intact heteroduplexes that still contain mismatch regions (i.e., regions that were either not nicked, cleaved, or degraded), can be transformed into a suitable host for optional repair of the mismatch regions, and expression.

For carrying out in vitro elongation, suitable polymerases include, for example, a Kornberg DNA polymerase I, a Klenow DNA polymerase I polymerase, a T4 DNA polymerase, a T7 DNA polymerase, a Taq DNA polymerase, a Micrococcal DNA polymerase, an alpha DNA polymerase, an AMV reverse transcriptase, an M-MuLV reverse transcriptase, an E. coli RNA polymerase, an SP6 RNA polymerase, a T3 RNA polymerase, a T7 RNA polymerase, an RNA polymerase II, or the like. In preferred embodiments, the polymerase lacks a strand displacement activity, such as, for example, a T4 polymerase, a T7 polymerase, and other non-strand displacing polymerases. Ligases that are suitable for use in the practice of the present invention include those that are well known in the art, such as, for example, a T4 RNA ligase, a T4 DNA ligase, an E. coli DNA ligase, and the like. The resulting chimeric nucleic acid sequences thus contain regions of crossovers.

The number of resulting crossovers incorporated in the progeny chimeric nucleic acid sequences can be defined and controlled such that all of the differences between the first and second parental single-stranded nucleic acids are incorporated into a single progeny chimeric nucleic acid sequence.

Even if a chimeric progeny sequence produced by these methods does not exhibit improved activity, the chimeric sequence can be optionally used as a diplomat sequence in other recombination reactions. As used herein, the term “diplomat sequence” refers to a nucleic acid sequence having an intermediate level of homology to each parental sequence to be recombined and thus facilitate cross-over events between the sequences and chimera formation. The use of diplomat sequences is further described in, e.g., “METHODS FOR MAKING CHARACTER STRINGS, POLYNUCLEOTIDES & POLYPEPTIDES HAVING DESIRED CHARACTERISTICS” by Selifonov and Stemmer, filed Feb. 5, 1999 (U.S. Ser. No. 60/118,854).

Single-stranded parental sequences can be prepared by any of the methods described herein for producing single stranded nucleic acid sequences. For example, the first or second parental single-stranded nucleic acids can be prepared by performing one or more cycles of an asymmetric polymerase chain reaction (e.g., with or without final addition of a double strand specific exonuclease, such as Exonuclease III). Optionally, the first or second parental single-stranded nucleic acids are provided by degrading specific single strands in double-stranded parental sequences with at least one nuclease (e.g., a Lambda exonuclease). Another option includes synthesizing the first or second parental single-stranded nucleic acids.

The hybridization, elongation, and/or ligation steps are typically carried out at the same temperature, although this is not required. The optimal temperature for carrying out the hybridization, elongation, and ligations steps can be readily determined by those having ordinary skill in the art, and will depend on the level of homology between first and second parental sequences, as well as the particular polymerase and/or ligase employed. The method can be readily carried out within a wide range of temperatures. For first and second parental nucleic acid sequences having relatively low level of homology with respect to each other (e.g., typically, about 70% or less, more typically about 60% or less, and usually about 50% or less) temperatures of about 45° C. or less, about 37° C. or less, about 25° C. or less, and even about 16° C. or less may be more suitable

The methods of generating chimeric progeny nucleic acids optionally include various downstream processing steps. For example, the chimeric progeny nucleic acids are typically amplified and/or expressed to provide at least one expression product. Expression products are optionally selected or screened for one or more desired traits or properties. Many suitable selecting and screening assays are described herein. The chimeric progeny nucleic acids are also optionally introduced into a cell, in which the introduced chimeric progeny nucleic acids are expressed to provide an expression product to the cell.

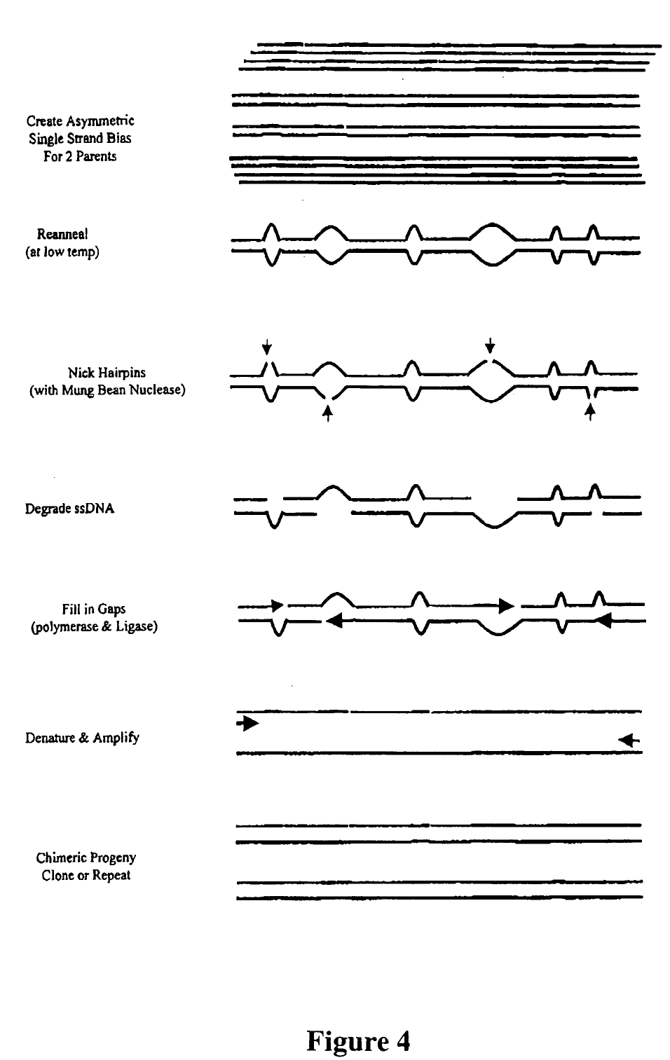

FIG. 4 schematically illustrates one embodiment of the methods of creating chimeric progeny by heteroduplex repair using Mung bean nucleases. As shown, asymmetric single-strand bias is created for two parents using, e.g., an asymmetric PCR. Single-strands of the two parental sequences are annealed at low temperature (e.g., 25° C.). In regions of sequence diversity between the two parent strands, the heteroduplex mismatch creates hairpin loops of nonhybridized sequences, which are nicked with a Mung bean nuclease. The level of nicking is typically controlled by varying the amount of nuclease used. Note, that overlapping regions of degradation will result in, e.g., truncated genes, but these are typically lost in subsequent amplification and cloning steps. Following strand nicking, a nuclease is generally used to cleave the nicked strands to produce sequence gaps, which are filled in using, e.g., a polymerase and a ligase to generate the chimeric progeny nucleic acids. Optional downstream steps include, e.g., amplifying or cloning the progeny, or repeating the method.

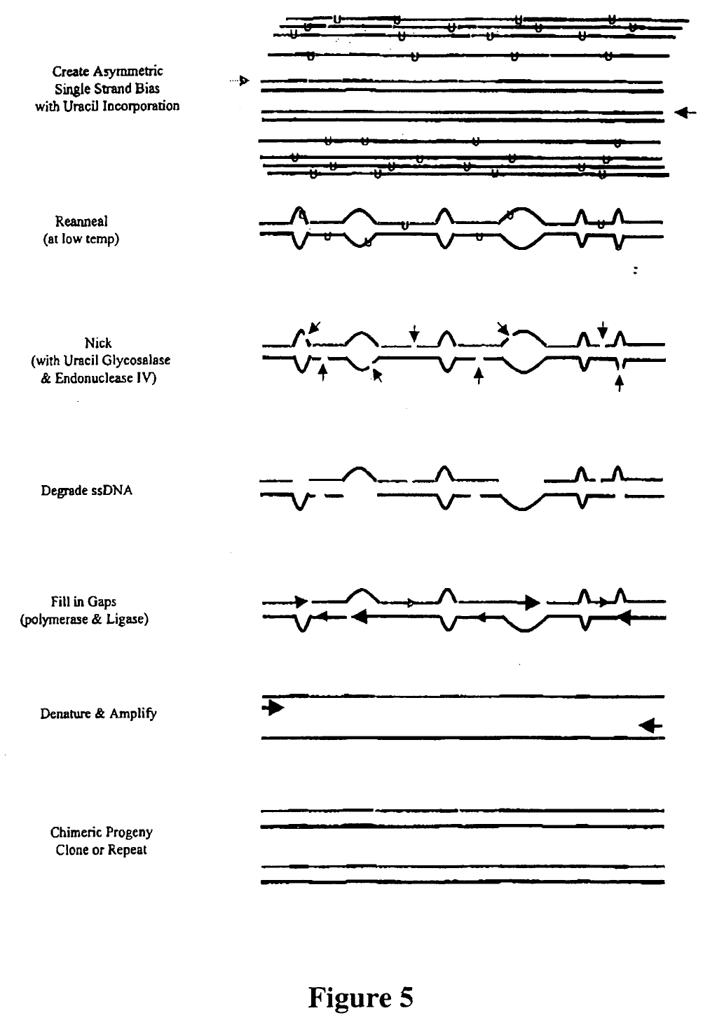

FIG. 5 schematically depicts one embodiment of the methods of creating chimeric progeny by heteroduplex repair that involve uracil incorporation. In this approach, asymmetric single strand bias is created with uracil incorporation and the resultant single-stranded parents are annealed at, e.g., room temperature. Again, the amount of uracil incorporated will determine the number of mismatch regions that are subsequently nicked. Heteroduplex mismatch regions that incorporate uracil are nicked using, e.g., Uracil Glycosylase and Endonuclease IV. Some of the nicks will be in heteroduplex mismatch regions and will result in single stranded ends. Nicks that result in hybridized regions will simply be repaired in the polymerase and ligation step. Following single strand degradation, sequence gaps are filled using, e.g., a polymerase and a ligase. As described above, the process can optionally be repeated to create more complex chimeras or the library of chimeric progeny can be cloned, expressed and screened.

Single-Stranded Nucleic Acid Template and Nucleic Acid Fragment Preparation

The methods of the present invention include using target sequences, such as single-stranded nucleic acid templates to mediate the isolation and/or recombination of a set of nucleic acid fragments. Single-stranded nucleic acid templates are selected from, e.g., sense cDNA sequences, antisense cDNA sequences, sense DNA sequences, antisense DNA sequences, sense RNA sequences, antisense RNA sequences, or the like. As illustrated above, each single-stranded nucleic acid template can also optionally include at least one affinity-label for use, e.g., in various separation steps of the invention. Additionally, single-stranded nucleic acid templates can include varying degrees of homology with corresponding target nucleic acid fragment populations to be isolated or recombined. Higher homology levels within a fragment pool can facilitate the polymerase-free recombination methods of the present invention. Many specific examples of target sequences for use in the methods described herein are described further below.

Single-stranded nucleic acid templates are prepared using various methods. One method for preparing single-stranded nucleic acid templates includes amplifying one or more double-stranded template nucleic acids in which each primer of a first of two primer sets comprises a 5′ terminal phosphate. Thereafter, one strand of each amplicon is degraded with a nuclease (e.g., a lambda exonuclease) in which the degraded strand includes the 5′ terminal phosphate, thus providing the single-stranded nucleic acid templates. The methods optionally include, e.g., synthesizing primers of the first primer set with the 5′ terminal phosphate, or phosphorylating a 5′ terminal of each member of the first primer set with, e.g., a kinase prior to the amplifying step. See, Higuchi and Ochman (1989) “Production of Single-Stranded DNA Templates by Exonuclease Digestion Following the Polymerase Chain Reaction,” Nucleic Acids Res. 17(14):5865. Another method for preparing single-stranded nucleic acid templates includes amplifying one or more double-stranded template nucleic acids in which each primer of a first of two primer sets comprises one or more 5′ terminal phosphorothioates. Following amplification, one strand of each amplicon is degraded with a nuclease (e.g., a T7 gene 6 exonuclease) in which the degraded strand lacks the one or more 5′ terminal phosphorothioates, thus providing the single-stranded nucleic acid templates. Each member of the first primer set typically includes 1, 2, 3, 4, 5, or more 5′ terminal phosphorothioates. See, Nikiforov et al. (1994) “The Use of Phosphorotioate Primers and Exonuclease Hydrolysis for the Preparation of Single-Stranded PCR Products and their Detection by Solid-Phase Hybridization,” PCR Methods and Applications 3:285-291. In another embodiment, nucleic acids are simply synthesized according to common available methods, which are discussed further below. Similarly, nucleic acids can be commercially ordered by one or skill, from any of a variety of commercial sources.

In another approach, single-stranded nucleic acid templates are obtained, e.g., from a double-stranded parental nucleic acid of interest, e.g., by digestion of a construct (e.g., a plasmid or the like) that includes the double-stranded parental nucleic acid insert, followed by, e.g., gel purification of the insert. Thereafter, the double-stranded parental nucleic acid insert is subjected to, e.g., recursive single primer extension in which the primer corresponds to either a sense or antisense sequence of the double-stranded parental insert. The extension reaction is conducted at a molar excess (e.g., about 30-fold) of the primer to double-stranded parental insert. Single strand amplification is performed by, e.g., about 10 reaction cycles (e.g., 30 seconds at 94° C., 30 seconds at 55° C., and one minute at 72° C.). Optionally, a two minute extension (e.g., incubation at 72° C.) is performed following the final cycle. The single-stranded product and template nucleic acids are isolated from other reaction components using, e.g., a Qiaex PCR clean-up kit (Qiagen, Inc.) or other method known in the art. The mixed population of nucleic acids is typically digested with, e.g., an appropriate restriction endonuclease, followed by, e.g., gel purification to obtain a pure population of single-stranded nucleic acids which corresponds to either the sense or antisense strand of the parental double-stranded parent.

As already discussed, the present invention also provides methods of preparing single-stranded nucleic acid fragments using a phagemid vector. In this approach, nucleic acids of interest are ligated into a phagemid (e.g., pGEM-T available from Promega) using a T-A cloning protocol (see, e.g., Zhou et al., (1995) Biotechniques 19:34-35 for cloning details) to generate phagemid derivatives bearing the nucleic acid of interest in either a sense or an antisense orientation with respect to the F1 origin of replication. Approaches described above can use double stranded nucleic acids (e.g., double stranded plasmid DNA) as the source of fragments. In contrast, phagemid-based technique often use single stranded phagemid DNA bearing the complement of the template as the source of nucleic acid fragments.

For example, if a phagemid construct that includes the antisense orientation of the nucleic acid of interest is selected as the source of single-strand nucleic acid template, other phagemids bearing sense orientations of the nucleic acid of interest are selected as sources of single-stranded nucleic acids to generate fragments that are complementary to the single-strand nucleic acid template. Thereafter, single-strand nucleic acids are prepared from the sense and antisense derivatives by, e.g., infecting cultures bearing the phagemids with helper phage (e.g., VCSM13 available from Stratagene) according to protocols known in the art. The resulting preparations of single-strand phagemid nucleic acids are digested with an appropriate restriction endocuclease. This digestion allows removal of unwanted double-strand phagemid nucleic acids from the samples and prevents the double-stranded phagemid nucleic acid from acting to reassemble the parental sequences. The sense strand derivatives are then fragmented with, e.g., DNase I, or by another method, and fragments (e.g., between about 25-75 bases) are gel-purified, phenol-chloroform extracted, ethanol precipitated, or the like.

As already discussed, the present invention also provides magnetic-based methods of isolating single-stranded nucleic acid templates. In this approach, one of two primers is synthesized with a 5′amino label (e.g. Aminolink, Clontech, Inc., Mountain View, Calif.) and followed by covalent coupling of the labeled primer to magnetic high density latex beads that are commercially available from many different sources. Following amplification in the presence of labeled and unlabeled primers, single-stranded nucleic acid templates that include the labeled primer are separated by magnetic separation at elevated temperatures, in which the labeled strand remains attached to a solid matrix or surface under application of a magnetic field while the other strand remains in solution.

Single-stranded nucleic acid templates are also optionally produced using selected nucleases. For example, certain exonucleases, such as Exonuclease III, Bal31, Mung bean nuclease, Lambda Exonucleoase, or the like are known to selectively degrade various forms of double stranded or partially double stranded nucleic acids (i.e., depending upon whether the double stranded nucleic acids include, e.g., 5′ overhangs or recesses, blunt 5′ ends, 3′ overhangs or recesses, or blunt 3′ ends). Nucleases can be used to selectively degrade double stranded nucleic acids such that the strand of interest is preserved. For example, ExoIII will progressively digest double stranded DNA starting from a blunt or recessed 3′ end, but not from a free single-stranded 3′ end. In one example, ExoIII is used to selectively degrade either the upper or lower strand of a nucleic acid duplex in which the non-degraded strand is protected by having a 3′ end that extends beyond the 5′ terminus of the opposite strand. This method is described further below.

In certain embodiments, RNA/DNA heteroduplexes can be used to generate single-stranded templates. For example, a gene, a pathway, a family or a fragment of a gene can be cloned into a vector for easy in vitro trancription of RNA corresponding to the target nucleic acid sequence. Transcripts are generated, e.g., using one of many commercially available in vitro transcription kits. The transcripts so generated are primed for second strand synthesis with an appropriately positioned primer and the second strand synthesized with reverse transcriptase. Reverse transcription provides single-stranded DNA from which the RNA can be selectively degraded using a variety of commercially available RNases (RNase A, RNase H, or the like).

The second set of nucleic acids can be derived from, e.g., cultured or uncultured microorganisms, complex biological mixtures (e.g. tissues, serum, pooled sera or tissues, multispecies consortia or the like), fossilized or other nonliving biological remains, environmental isolates (e.g. from soil, groundwater, waste facilities, deep-sea or other extreme environments), consensus populations computer-modeled nucleic acids, artificially selected sequences or the like. The second set of nucleic acids can also be derived from, e.g., individual cDNA molecules, cloned sets of cDNAs, cDNA libraries; extracted, natural and/or in vitro transcribed RNAs; or characterized, uncharacterized and cloned genomic DNA and genomic DNA libraries by enzymatic digestion, chemical or physical fragmentation or equivalent methods for providing a pool of gene fragments. Methods of isolating DNA or RNA are well-known. See e.g., Sambrook, Ausubel, and Berger, infra. Optionally, the first set of nucleic acids (e.g., the single-stranded nucleic acid templates) is also derived from the same sources as the second set of nucleic acids.

Nucleic acid fragment sizes typically vary according to, e.g., the size of the single-stranded nucleic acid template being used. Although any fragment size can be used, the methods of the invention generally include fragment sizes that are smaller on average than the corresponding single-stranded nucleic acid template. For example, in certain embodiments, fragments include about 1000 or fewer bases, more typically about 500 bases or less, sometimes about 100 bases or less, or, e.g., about 50, 25, 10 or fewer bases.

In one embodiment, a double stranded fragment pool is optionally prepared by initially preparing double stranded plasmid nucleic acids using, e.g., a commercial plasmid isolation kit (e.g., a Qiagen Maxi plasmid isolation kit). Once double stranded plasmids are obtained, trial fragmentation reactions (e.g., 1, 2, 3, 4, 5, or more) are typically performed using various amounts (e.g., 0, 0.1, 0.2, 0.5, 0.8 ml or the like) of a selected nuclease (e.g., an DNAse or a RNAse). For example, each selected amount of nuclease can be reacted with about 2 μg of the plasmid in about 20 μl of 50 mM Tris-Cl and 10 mM MnCl₂ at pH 7.5. Each reaction mixture is incubated for about 10 minutes at room temperature. Nuclease digestion is generally stopped by, e.g., being placed on ice along with the addition of about 1 μl of 0.5 M EDTA at pH 8.0. The reaction products are typically assessed using a preparative gel (e.g., 1.5% agarose/1× TBE), column, or other common method, e.g., with appropriate markers of between about 100-1000 base pairs. Typically, the reaction conditions yielding between about 50-500 base pair fragments are then identified, and a double stranded plasmid sample (e.g., about 20 μg) is digested using those conditions. Following digestion, the fragments are separated by electrophoresis (e.g., a 0.7% agarose/1×TBE preparative gel) or the like. Fragments of between about 50-500 base pairs are typically isolated and purified from the gel using, e.g., Whatman glass micro-fiber filter paper and a dialysis membrane. The purified fragments are typically subjected to purification, e.g., using phenol extraction and ethanol precipitation, washing in 70% EtOH, air drying, etc. Thereafter, the fragments (e.g., 1 μg) are generally resuspended in a useful buffer, e.g., TE.

Alternatively, nucleic acid fragments can be generated from single stranded phagemid DNA prepared as described herein and fragmented by physical (e.g., physical shearing), chemical, or enzymatic (e.g., digestion of double stranded or single stranded nucleic acid, such as by a DNase or an RNase) approaches. As noted, the ability to use double stranded nucleic acid populations as sources of fragments introduces versatility into the technique by allowing both in vitro, in vivo and synthetic methods of DNA preparation to be used. Furthermore, in preparative methods involving amplification or other use of synthetic primers, it can be advantageous to prepare phosphorylated primers when subsequent high efficiency ligation is desired. The fragment population is also provided by various other alternatives including, e.g., direct synthesis of either single or double stranded DNA sequences, direct extraction from environmental or uncharacterized biological materials, packaging of single stranded phagemids, selective strand degradation, magnetic separation methods, and many techniques.

As mentioned, the nucleic acid fragments used in the methods of recombination or of nucleic acid fragment isolation can include a standardized (or “normalized”) or a non-standardized set of nucleic acids. Populations of nucleic acids are typically normalized to prevent a few fragments from dominating the hybridization properties of a complex mixture by shear abundance or overrepresentation. Methods for normalization are known in the art. See, e.g., U.S. Pat. No. 6,001,574 “PRODUCTION AND USE OF NORMALIZED DNA LIBRARIES” issued Dec. 14, 1999 to Short, J. M and Mathur, E. J.

In general, the preparation of target sequences can include certain DNA synthetic techniques (e.g., mononucleotide- and/or trinucleotide-based synthesis, reverse-transcription, etc.), cloning, DNA amplification, nuclease digestion, etc. Searchable sequence information available from nucleic acid databases can also be utilized during the nucleic acid sequence selection and/or design processes. Genbank®, Entrez®, EMBL, DDBJ, GSDB, NDB and the NCBI are examples of public database/search services that can be accessed. These databases are generally available via the internet or on a contract basis from a variety of companies specializing in genomic information generation and/or storage. These and other helpful resources are readily available and known to those of skill.

The sequence of a polynucleotide to be used in any of the methods of the present invention can also be readily determined using techniques well-known to those of skill, including Maxam-Gilbert, Sanger Dideoxy, and Sequencing by Hybridization methods. For general descriptions of these processes consult, e.g., Stryer, L., Biochemistry (4^{th} Ed.) W.H. Freeman and Company, New York, 1995 (Stryer) and Lewin, B. Genes VI Oxford University Press, Oxford, 1997 (Lewin). See also, Maxam, A. M. and Gilbert, W. (1977) “A New Method for Sequencing DNA,” Proc. Natl. Acad. Sci. 74:560-564, Sanger, F. et al. (1977) “DNA Sequencing with Chain-Terminating Inhibitors,” Proc. Natl. Acad. Sci. 74:5463-5467, Hunkapiller, T. et al. (1991) “Large-Scale and Automated DNA Sequence Determination,” Science 254:59-67, and Pease, A. C. et al. (1994) “Light-Generated Oligonucleotide Arrays for Rapid DNA Sequence Analysis,” Proc. Natl. Acad. Sci. 91:5022-5026. Furthermore, commercially available services provide sequencing, nucleic acid synthesis and the like.

When recombining homologous sequences, e.g., nucleic acid fragments using single-stranded templates or other downstream processing steps following recombination, the present invention optionally includes aligning homologous nucleic acid sequences or regions of similarity. For example, in one aspect, the invention relates to a method of recombining nucleic acid fragments having high sequence homology with a single-stranded template using only a ligase (i.e., polymerase-free recombination) to fill in sequence gaps (e.g., from about one to about five nucleotides) and/or at least covalently link at least two parental nucleic acid fragments. Homology can be assessed, e.g., by aligning homologous nucleic acid sequences (e.g., in a computer) to select conserved regions of sequence identity and regions of sequence diversity. Suitable nucleic acid fragment populations can then be, e.g., synthesized to provide sufficient homology based upon data derived from such sequence alignments. Similarly, an aspect of the invention can include deriving the sequences of an additional set of nucleic acid fragments from, e.g., isolated nucleic acid fragments or chimeric nucleic acid sequences generated by the methods of the present invention, for subsequent downstream recombination by aligning the fragments or chimeric sequences to identify regions of identity and regions of diversity.

In the processes of sequence comparison and homology determination, one sequence, e.g., one fragment or subsequence of a gene sequence to be recombined, can be used as a reference against which other test nucleic acid sequences are compared. This comparison can be accomplished with the aid of a sequence comparison instruction set, i.e., algorithm, or by visual inspection. When an algorithm is employed, test and reference sequences are input into a computer, subsequence coordinates are designated, as necessary, and sequence algorithm program parameters are specified. The algorithm then calculates the percent sequence identity for the test nucleic acid sequence(s) relative to the reference sequence, based on the specified program parameters. Among other things, a sequence comparison algorithm can provide sets of nucleic acid sequences to be synthesized and used to facilitate, e.g., single-strand mediated recombination or downstream recombination processes. Integrated systems that are relevant to the invention are discussed further below.

For purposes of the present invention, suitable sequence comparisons can be executed, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection. See generally, Current Protocols in Molecular Biology, F. M. Ausubel et al., eds., Current Protocols, a joint venture between Greene Publishing Associates, Inc. and John Wiley & Sons, Inc., (supplemented through 1999).

One example search algorithm that is suitable for determining percent sequence identity and sequence similarity is the Basic Local Alignment Search Tool (BLAST) algorithm, which is described in Altschul et al., J. Mol. Biol. 215:403-410 (1990). Software for performing BLAST analyses is publicly available through the National Center for Biotechnology Information (http://www.ncbi.nlm.nih.gov/).

After sequence information has been obtained as described above, that information can be used to design and synthesize target nucleic acid sequences corresponding to, e.g., the single-stranded nucleic acid templates or the nucleic acid fragment populations (e.g., for single-strand-mediated recombination, or for other approaches, such as oligonucleotide and in silico recombination which are discussed below). These sequences can be synthesized utilizing various solid-phase strategies involving mononucleotide- and/or trinucleotide-based phosphoramidite coupling chemistry. In these approaches, nucleic acid sequences are synthesized by the sequential addition of activated monomers and/or trimers to an elongating polynucleotide chain. See e.g., Caruthers, M. H. et al. (1992) Meth. Enzymol. 211:3-20.

In the formats involving trimers, trinucleotide phosphoramidites representing codons for all 20 amino acids are used to introduce entire codons into the growing oligonucleotide sequences being synthesized. The details on synthesis of trinucleotide phosphoramidites, their subsequent use in oligonucleotide synthesis, and related issues are described in, e.g., Virnekäs, B., et al. (1994) Nucleic Acids Res., 22, 5600-5607, Kayushin, A. L. et al. (1996) Nucleic Acids Res., 24, 3748-3755, Huse, U.S. Pat. No. 5,264,563 “PROCESS FOR SYNTHESIZING OLIGONUCLEOTIDES WITH RANDOM CODONS,” Lyttle et al., U.S. Pat. No. 5,717,085 “PROCESS FOR PREPARING CODON AMIDITES,” Shortle et al., U.S. Pat. No. 5,869,644 “SYNTHESIS OF DIVERSE AND USEFUL COLLECTIONS OF OLIGONUCLEOTIDES,” Greyson, U.S. Pat. No. 5,789,577 “METHOD FOR THE CONTROLLED SYNTHESIS OF POLYNUCLEOTIDE MIXTURES WHICH ENCODE DESIRED MIXTURES OF PEPTIDES,” and Huse, WO 92/06176 “SURFACE EXPRESSION LIBRARIES OF RANDOMIZED PEPTIDES.”

The chemistry involved in these synthetic methods is known by those of skill. In general, they utilize phosphoramidite solid-phase chemical synthesis in which the 3′ ends of nucleic acid substrate sequences are covalently attached to a solid support, e.g., controlled pore glass. The 5′ protecting groups can be, e.g., a triphenylmethyl group, such as, dimethoxyltrityl (DMT) or monomethyoxytrityl, a carbonyl-containing group, such as, 9-fluorenylmethyloxycarbonyl (FMOC) or levulinoyl, an acid-cleavable group, such as, pixyl, a fluoride-cleavable alkylsilyl group, such as, tert-butyl dimethylsilyl (T-BDMSi), triisopropyl silyl, or trimethylsilyl. The 3′ protecting groups can be, e.g., β-cyanoethyl groups.

These formats can optionally be performed in an integrated automated synthesizer system that automatically performs the synthetic steps. See also, Integrated Systems, infra. This aspect includes inputting character string information into a computer, the output of which then directs the automated synthesizer to perform the steps necessary to synthesize the desired nucleic acid sequences. Automated synthesizers are available from many commercial suppliers including PE Biosystems and Beckman Instruments, Inc.

To further ensure that target nucleic acid or gene sequences, e.g., single-stranded nucleic acid templates or nucleic acid fragments are ultimately obtained, certain techniques can be utilized following DNA synthesis. For example, gel purification is one method that can be used to purify synthesized polynucleotides. High-performance liquid chromatography (HPLC) can be similarly employed. Furthermore, translational coupling can be used to assess gene functionality, e.g., to test whether full-length sequences such as full-length single-stranded nucleic acid templates, e.g., that correspond to a selected gene are generated. In this process, the translation of a reporter protein, e.g., green fluorescent protein or β-galactosidase is coupled to that of the target gene product. This enables one to distinguish, e.g., full-length enzyme sequences from those that contain deletions or frame shifts.

In lieu of synthesizing the desired sequences, essentially any nucleic acid can optionally be custom ordered from any of a variety of commercial sources, such as The Midland Certified Reagent Company (mcrc@oligos.com), The Great American Gene Company (www.genco.com), ExpressGen, Inc. (www.expressgen.com), Operon Technologies, Inc. (www.operon.com), and many others.

Target nucleic acid sequences, such as the single-stranded templates or the nucleic acid sequences to be fragmented, or the fragments themselves, can be derived from expression products, e.g., mRNAs expressed from genes within a cell of a plant or other organism, or from genomic DNA, cDNA libraries or the like. For example, a number of techniques are available for isolating and detecting RNAs. For example, northern blot hybridization is widely used for RNA detection, and is generally taught in a variety of standard texts on molecular biology, including Current Protocols in Molecular Biology, F. M. Ausubel et al., eds., Current Protocols, a joint venture between Greene Publishing Associates, Inc. and John Wiley & Sons, Inc., (supplemented through 1999) (Ausubel), Sambrook et al., Molecular Cloning—A Laboratory Manual (2nd Ed.), Vol. 1-3, Cold Spring Harbor Laboratory, Cold Spring Harbor, N.Y., 1989 (Sambrook), and Berger and Kimmel, Guide to Molecular Cloning Techniques, Methods in Enzymology volume 152 Academic Press, Inc., San Diego, Calif. (Berger). Furthermore, one of skill will appreciate that essentially any RNA can be converted into a double stranded DNA using a reverse transcriptase enzyme and a polymerase. See, Ausubel, Sambrook and Berger. Messenger RNAs can be detected by converting, e.g., mRNAs into cDNAs, which are subsequently detected in, e.g., a standard “Southern blot” format.

Examples of techniques sufficient to direct persons of skill through in vitro amplification methods, useful e.g., for amplifying synthesized template strands and nucleic acid fragments, or in certain downstream amplifying steps involving, e.g., chimeric nucleic acid sequences and isolated nucleic acid fragments, include the polymerase chain reaction (PCR), the ligase chain reaction (LCR), Qβ-replicase amplification, and other RNA polymerase mediated techniques (e.g., NASBA). These techniques are found in Ausubel, Sambrook, and Berger, as well as in Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; Kwoh et al. (1989) Proc. Nail. Acad. Sci. USA 86, 1173; Guatelli et al. (1990) Proc. Natl. Acad. Sci. USA 87, 1874; Lomell et al. (1989) J. Clin. Chem 35, 1826; Landegren et al. (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids, e.g., full-length chimeric nucleic acid sequences other nucleic acid sequences, by PCR are summarized in Cheng et al. (1994) Nature 369: 684-685 and the references therein, in which PCR amplicons of up to 40 kb are generated.

In one preferred method, assembled sequences are checked, e.g., for incorporation of specific subsequences of genes. This can be done by cloning and sequencing the nucleic acids, and/or by restriction digestion, e.g., as essentially taught in Ausubel, Sambrook, and Berger, supra. In addition, sequences can be PCR amplified and sequenced directly. Thus, in addition to, e.g., Ausubel, Sambrook, Berger, and Innis, additional PCR sequencing methodologies are also particularly useful. For example, direct sequencing of PCR generated amplicons by selectively incorporating boronated nuclease resistant nucleotides into the amplicons during PCR and digestion of the amplicons with a nuclease to produce sized template fragments has been performed (Porter et al. (1997) Nucleic Acids Res. 25(8): 1611-1617).

Single-Stranded Nucleic Acid Template and Nucleic Acid Fragment Sources

Essentially any nucleic acid can be modified using the methods described herein. Common sequence repositories for known proteins include GenBank, EMBL, DDBJ and the NCBI. Other repositories can easily be identified by searching the internet. Suitable nucleic acids include those that are commercially available. Specific target sequences of interest typically include commercially important coding sequences or sequences complementary thereto. These include, e.g., various pharmaceutically, agriculturally, and/or industrially relevant nucleic acids, including those noted above (and in the references herein) and those described herein below. The exemplary enzymes listed herein, and sequences corresponding to them, are offered to illustrate but not to limit the present invention. Additional sequences corresponding to these and to other potential targets are known in the art and are readily obtainable by cloning, PCR, synthesis or the like. Any of the following proteins, nucleic acids, enzymes, pathways, or other systems can be modified, produced, or otherwise developed according to the methods herein. For example, any of the proteins, nucleic acids, enzymes, pathways, or other systems can be modified via the single-strand mediated recombination methods herein, or any other method described herein.

Pharmaceutically-Related Parental Nucleic Acids and Expression Products

One class of parental nucleic acid sequences well suited for use as substrates in the methods described herein include those encoding expression products with at least potential pharmaceutical relevance. These expression products include, e.g., therapeutic proteins, transcriptional and expression activators, vaccines, small proteins, antibodies, or the like. Some specific examples of these molecules are described further below.

Therapeutic Proteins

Suitable targets for use in the methods of the invention include nucleic acids encoding therapeutic proteins such as erythropoietin (EPO), insulin, peptide hormones such as human growth hormone, growth factors and cytokines such as epithelial Neutrophil Activating Peptide-78, GROα/MGSA, GROβ, GRO, MIP-1α, MIP-1, MCP-1, epidermal growth factor, fibroblast growth factor, hepatocyte growth factor, insulin-like growth factor, the interferons, the interleukins, keratinocyte growth factor, leukemia inhibitory factor, oncostatin M, PD-ECSF, PDGF, pleiotropin, SCF, c-kit ligand, VEGEF, G-CSF etc. Many of these proteins are commercially available (See, e.g., the Sigma BioSciences 1997 catalogue and price list), and the corresponding genes are well-known.

Transcriptional and Expression Activators

Another class of preferred targets are transcriptional and expression activators. Example transcriptional and expression activators include genes and proteins that modulate cell growth, differentiation, regulation, or the like. Expression and transcriptional activators are found in prokaryotes, viruses, and eukaryotes, including fungi, plants, and animals, including mammals, providing a wide range of therapeutic targets. It will be appreciated that expression and transcriptional activators regulate transcription by many mechanisms, e.g., by binding to receptors, stimulating a signal transduction cascade, regulating expression of transcription factors, binding to promoters and enhancers, binding to proteins that bind to promoters and enhancers, unwinding DNA, splicing pre-mRNA, polyadenylating RNA, and degrading RNA. Expression activators include cytokines, inflammatory molecules, growth factors, their receptors, and oncogene products, e.g., interleukins (e.g., IL-1, IL-2, IL-8, etc.), interferons, FGF, IGF-I, IGF-II, FGF, PDGF, TNF, TGF-α, TGF-β, EGF, KGF, SCF/c-Kit, CD40L/CD40, VLA-4/VCAM-1, ICAM-1/LFA-1, and hyalurin/CD44; signal transduction molecules and corresponding oncogene products, e.g., Mos, Ras, Raf, and Met; and transcriptional activators and suppressors, e.g., p53, Tat, Fos, Myc, Jun, Myb, Rel, and steroid hormone receptors such as those for estrogen, progesterone, testosterone, aldosterone, the LDL receptor ligand and corticosterone. RNases such as Onconase and EDN are also preferred targets. Any of these proteins or corresponding nucleic acids can be made, modified, evolved or otherwise developed according to the methods described herein.

Vaccines

Nucleic acids encoding proteins from, e.g., infectious organisms can be recombined according to the methods described herein, e.g. for vaccine and other applications, including those from, infectious fungi, e.g., Aspergillus, Candida species; bacteria, particularly E. coli, which serves a model for pathogenic bacteria, as well as medically important bacteria such as Staphylococci (e.g., aureus), Streptococci (e.g., pneumoniae), Clostridia (e.g., perfringens), Neisseria (e.g., gonorrhoea), Enterobacteriaceae (e.g., coli), Helicobacter (e.g., pylori), Vibrio (e.g., cholerae), Campylobacter (e.g., jejuni), Pseudomonas (e.g., aeruginosa), Haemophilus (e.g., influenzae), Bordetella (e.g., pertussis), Mycoplasma (e.g., pneumoniae), Ureaplasma (e.g., urealyticum), Legionella (e.g., pneumophilia), Spirochetes (e.g., Treponema, Leptospira, and Borrelia), Mycobacteria (e.g., tuberculosis, smegmatis), Actinomyces (e.g., israelii), Nocardia (e.g., asteroides), Chlamydia (e.g., trachomatis), Rickettsia, Coxiella, Ehrilichia, Rocholimaea, Brucella, Yersinia, Francisella, and Pasteurella; protozoa such as sporozoa (e.g., Plasmodia), rhizopods (e.g., Entamoeba) and flagellates (Trypanosoma, Leishmania, Trichomonas, Giardia, etc.); viruses such as (+) RNA viruses (examples include Poxviruses e.g., vaccinia; Picornaviruses, e.g. polio; Togaviruses, e.g., rubella; Flaviviruses, e.g., HCV; and Coronaviruses), (−) RNA viruses (examples include Rhabdoviruses, e.g., VSV; Paramyxovimses, e.g., RSV; Orthomyxovimses, e.g., influenza; Bunyaviruses; and Arenaviruses), dsDNA viruses (Reoviruses, for example), RNA to DNA viruses, i.e., Retroviruses, e.g., especially HIV and HTLV, and certain DNA to RNA viruses such as Hepatitis B virus. Any of these can be made, modified or developed according to the methods described herein.

Small Proteins

Small proteins such as defensins (antifungal proteins of about 50 amino acids, EF40 (an anti fungal protein of 28 amino acids), peptide antibiotics, and peptide insecticidal proteins are also targets and exist as families of related proteins which can be used to provide templates, parental nucleic acids, or fragments according to the present invention. Any of these proteins or corresponding nucleic acids can be made, modified, evolved or otherwise developed according to the methods described herein.

Antibodies

In another application, antibody genes are recombined according to the methods of the invention. For example, a wide variety of antibodies and antibody genes which can be recombined by the methods herein are set forth in U.S. Ser. No. 60/176,002, “ANTIBODY SHUFFLING” by Karrer et al. Any of these can be made, modified or developed according to the methods described herein.

Other Targets

Preferred known genes/proteins suitable for modification according to the methods herein also include the following: Alpha-i antitrypsin, Angiostatin, Antihemolytic factor, Apolipoprotein, Apoprotein, Atrial natriuretic factor, Atrial natriuretic polypeptide, Atrial peptides, C—X—C chemokines (e.g., T39765, NAP-2, ENA-78, Gro-a, Gro-b, Gro-c, IP-10, GCP-2, NAP-4, SDF-1, PF4, MIG), Calcitonin, CC chemokines (e.g., Monocyte chemoattractant protein-1, Monocyte chemoattractant protein-2, Monocyte chemoattractant protein-3, Monocyte inflammatory protein-1 alpha, Monocyte inflammatory protein-1 beta, RANTES, 1309, R83915, R91733, HCC1, T58847, D31065, T64262), CD40 ligand, Collagen, Colony stimulating factor (CSF), Complement factor 5a, Complement inhibitor, Complement receptor 1, Factor IX, Factor VII, Factor VIII, Factor X, Fibrinogen, Fibronectin, Glucocerebrosidase, Gonadotropin, Hedgehog proteins (e.g., Sonic, Indian, Desert), Hemoglobin (for blood substitute; for radiosensitization), Hirudin, Human serum albumin, Lactoferrin, Luciferase, Neurturin, Neutrophil inhibitory factor (NIF), Osteogenic protein, Parathyroid hormone, Protein A, Protein G, Relaxin, Renin, Salmon calcitonin, Salmon growth hormone, Soluble complement receptor I, Soluble I-CAM 1, Soluble interleukin receptors (IL-1, 2, 3, 4, 5, 6, 7, 9, 10, 11, 12, 13, 14, 15), Soluble TNF receptor, Somatomedin, Somatostatin, Somatotropin, Streptokinase, Superantigens, i.e., Staphylococcal enterotoxins (SEA, SEB, SEC1, SEC2, SEC3, SED, SEE), Toxic shock syndrome toxin (TSST-1), Exfoliating toxins A and B, Pyrogenic exotoxins A, B, and C, and M. arthritides mitogen, Superoxide dismutase, Thymosin alpha 1, Tissue plasminogen activator, Tumor necrosis factor beta (TNF beta), Tumor necrosis factor receptor (TNFR), Tumor necrosis factor-alpha (TNF alpha) and Urokinase. Any of these can be made, modified or developed according to the methods described herein.

Agriculturally-Related Parental Nucleic Acids and Expression Products

Other proteins relevant to non-medical uses, such as inhibitors of transcription or toxins of crop pests, e.g., insects, fungi, weed plants, and the like, are also preferred targets for recombination by one or more of the methods herein. Many agriculturally-related target sequences which are suitably used in the methods of the invention are disclosed in a variety of patent-related publications and the references noted herein, including, e.g., WO 00/09727 “DNA Shuffling to Produce Herbicide Selective Crops;” WO 99/57128 “Optimization of Pest Resistance Genes Using Shuffling;” U.S. Ser. No. 60/167,452 “Shuffling of Agrobacterium and Viral Genes, Plasmids and Genomes for Improved Plant Transformation;” WO 00/20573 “DNA Shuffling to Produce Nucleic Acids for Mycotoxin Detoxification;” WO 00/28018 “Modified ADP-Glucose Pyrophosphorylase for Improvement and Optimization of Plant Phenotypes;” WO 00/28017 “Modifed Phosphoenoylpyruvate Carboxylase for Improvement and Optimization of Plant Phenotypes;” WO 00/28008 “Modified Ribulose 1,5-Bisphosphate Carboxyl ase/Oxygenase;” PCT/US00/09285 “Modified Lipid Production;” PCT/US00/09840 “Modified Starch Metabolism Enzymes and Encoding Genes for Improvement and Optimization of Plant Phenotypes;” and U.S. Ser. No. 60/202,233 “Evolution of Plant Disease Response Pathways to Enable the Development of Plant Based Biological Sensors and to Develop Novel Disease Resistance Strategies;” which are each incorporated by reference herein in their entirety for all purposes. Any of these can be made, modified or developed according to the methods described herein.

Herbicide Resistance/Selectivity

For example, WO 00/09727 “DNA Shuffling to Produce Herbicide Selective Crops” describes the use of various diversity generation methods, including recombination, mutation and the like, e.g., in combination with various exemplar selection methods, for modifying genes that have (or even which can be modified to have) herbicide resistance/selectivity. The targets and selection assays noted in this case (e.g., genes that are recombined to provide herbicide selectivity and/or resistance and assays used to detect these properties) are also suitable for use in the methods described herein. For example, the targets for diversity generation noted in WO 00/09727 can be used as template nucleic acids, or can be digested and hybridized to template nucleic acids or otherwise used in the methods noted herein. The selection assays for selecting for desirable activities as taught in WO 00/09727 can be used to select for new or improved properties of interest following application of the methods described. Any of these can be made, modified or developed according to the methods described herein.

For example, two major classes of enzymes involved in conferring natural crop selectivity to herbicides are (a) monooxygenases such as cytochrome P450 monooxygenases (P450s) and (b) glutathione sulfur-transferases (GSTs) and homoglutathione sulfur-transferases (HGSTs). Several hundred cytochrome P450 genes, which encode enzymes that mediate a variety of chemical processes in the cell, have been cloned or otherwise characterized. For an introduction to cytochrome P450, see, Ortiz de Montellano (ed.) (1995) Cytochrome P450 Structure Mechanism and Biochemistry, Second Edition Plenum Press (New York and London) (“Ortiz de Montellano, 1995”) and the references cited therein.

Thus, exemplar parental nucleic acids for modification according to the methods of the invention include genes encoding P450 monooxygenases, glutathione sulfur transferases, homoglutathione sulfur transferases, glyphosate oxidases, phosphinothricin acetyl transferases, dichlorophenoxyacetate monooxygenases, acetolactate synthases, 5-enol pyruvylshikimate-3-phosphate synthases, and UDP-N-acetylglucosamine enolpyruvyltransferases. The choice of parental nucleic acid may depend in part on the specificity of herbicide tolerance desired with respect to the expression product of the progeny chimeric nucleic acid. For example, P450 monooxygenase genes from corn and wheat encode activities which confer tolerance to the herbicide dicamba, making these genes suitable targets for recombination. Other candidate nucleic acids include, for example, glutathione sulfur transferase genes from maize, homoglutathione sulfur transferase genes from soybean, glyphosate oxidase genes from bacteria, phosphinothricin acetyl transferase genes from bacteria, dichlorophenoxyacetate monooxygenase genes from bacteria, acetolactate synthase genes from plants, protoporphyrinogen oxidase genes from plants and algae, 5-enolpyruvylshikimate-3-phosphate synthase genes from plants and bacteria, and UDP-N-acetylglucosamine enolpyruvyltransferase genes from bacteria.

One target, Acetolactate synthase (ALS; also known as acetohydroxyacid synthase or AHAS) is involved in the plant branched-chain amino acid biosynthetic pathway. ALS is inhibited by and is the target site for herbicides such as sulphonylureas, imidazolinones, and triazolopyrimidines. ALS sequences from Arabidopsis (GenBank accession T20822), cotton (GenBank accession Z46960), barley (GenBank accession AF059600) and other plant and non-plant sources are available and can be used to, e.g., synthesize nucleic acids for use as recombination substrates, or as probes for isolation of ALS genes from other sources.

In general, as with all targets noted herein, allelic and interspecific variants of a parental nucleic acid or mutated or otherwise engineered nucleic acids can be employed in the invention methods described herein. Variant forms produced by recursive recombination, chemically synthesizing a plurality of nucleic acids homologous to the parental nucleic acid, produced by error-prone transcription of the parental nucleic acid, produced by replication of the parental nucleic acid in a mutator cell strain or the like, can also be used in the methods described herein. Any other source for nucleic acid starting materials, as noted herein, in the references noted herein, or as otherwise noted in the art, can be used in the methods described herein.

A variety of screening methods can be used to screen recombinant chimeric nucleic acids produced by the invention methods, including those described in WO 99/57128. In this example, the precise screen that is used depends on the herbicide against which a library of variant chimeric nucleic acids is selected. By way of example, the library to be screened can be present in a population of cells. The library is screened by growing the cells in or on a medium comprising the herbicide and selecting for a detected physical difference between the herbicide and a modified form of the herbicide in the cell. Exemplary herbicides include dicamba, glyphosate, bisphosphonates, sulfentrazones, imidazolinones, sulfonylureas, and triazolopyrimidines. For example, oxidation of the herbicide can be monitored, preferably by spectroscopic methods, thereby providing a measure of how effective the activities encoded by the library are at metabolizing the herbicide. Similarly, glutathione conjugation to an herbicide or herbicide metabolite, or homoglutathione conjugation to an herbicide or herbicide metabolite can also be selected for, based upon a difference in the physical properties of an herbicide before and after conjugation. Alternatively, the library is screened by growing the cells in or on a medium comprising the herbicide and selecting for enhanced growth of the cells in the presence of the herbicide. Enhanced growth of the cell could require the presence of the activity encoded by the recombinant herbicide tolerance nucleic acid. In one variation, the encoded activity is a herbicide metabolic activity, and the cells require the metabolic product of the herbicide for growth. Herbicide tolerance activity to more than one herbicide can simultaneously be screened or selected for in a library, i.e., with the goal of identifying a recombinant herbicide tolerance nucleic acid (or nucleic acids) that encode tolerance activities to more than one herbicide.

Iterative screening and selection for the activities noted herein, including herbicide tolerance and the other targets herein, is also a feature of the invention. In these methods, a chimeric nucleic acid identified as conferring, e.g., an herbicide tolerance activity to a cell can be further modified, e.g., by recombination, either with parental nucleic acids, or with other nucleic acids (e.g., variant forms of the parental nucleic acid), e.g., as templates or fragments, to produce a second library or nucleic acid set. The second library is then screened, e.g., in the case of herbicide activity, for one or more herbicide tolerance activity, which can be a tolerance activity to the same herbicide as in the first round of screening, or to a different herbicide. This process can be optionally iteratively repeated as many times as desired, until a recombinant herbicide tolerance chimeric nucleic acid with optimized properties is obtained. If desired, recombinant herbicide tolerance chimeric nucleic acids identified by any of the methods described herein can be cloned and, optionally, expressed. For example, the chimeric nucleic acid can be transduced into a plant to confer a herbicide tolerance activity to the plant. If desired, herbicide tolerance activity conferred to the plant can be tested, e.g., by field testing the herbicide tolerance of the plant.

Insect Resistance

Other suitable target nucleic acids for recombination/selection in the methods herein include insect resistance genes, such as those described in WO 99/57128 “Optimization of Pest Resistance Genes Using Shuffling.” These genes can be used as template nucleic acids, or can be digested and hybridized to template nucleic acids or otherwise used in the methods as noted herein. Selection assays suitable for use in the practice of the present invention for selecting for desirable activities include those described in WO 99/57128. Exemplar pest resistance genes suitable for use in the practice of the present invention include Bt toxins, including one or more of: cry1Aa1, cry1Aa2, cry1Aa3, cry1Aa4, cry1Aa5, cry1Aa6, cry1Ab1, cry1Ab2, cry1Ab3, cry1Ab4, cry1Ab5, cry1Ab6, cry1Ab7, cry1Ab8, cry1Ab9, cry1Ab10, cry1Ac1, cry1Ac2, cry1Ac3, cry1Ac4, cry1Ac5, cry1Ac6, cry1Ac7, cry1Ac8, cry1Ac9, cry1Ac10, cry1Ad1, cry1Ae1, cry1Af1, cry1Ba1, cry1Ba2, cry1Bb1, cry1Bc1, cry1Bd1, cry1Ca1, cry1Ca2, cry1Ca3, cry1Ca4, cry1Ca5, cry1Ca6, cry1Ca7, cry1Cb1, cry1Da1, cry1Db1, cry1Ea1, cry1Ea2, cry1Ea3, cry1Ea4, cry1Eb1, cry1Fa1, cry1Fa2, cry1Fb1, cry1Fb2, cry1Ga1, cry1Ga2, cry1Gb1, cry1Ha1, cry1Hb1, cry1Ia1, cry1Ia2, cry1Ia3, cry1Ia4, cry1Ia5, cry1Ib1, cry1Ic1, cry1Ja1, cry1Jb1, cry1Ka1, cry2Aa1, cry2Aa2, cry2Aa3, cry2Aa4, cry2Ab1, cry2Ab2, cry2Ac1, cry3Aa1, cry3Aa2, cry3Aa3, cry3Aa4, cry3Aa5, cry3Aa6, cry3Ba1, cry3Ba2, cry3Bb1, cry3Bb2, cry3Ca1, cry4Aa1, cry4Aa2, cry4Ba1, cry4Ba2, cry4Ba3, cry4Ba4, cry5Aa1, cry5Ab1, cry5Ac1, cry5Ba1, cry6Aa1, cry6Ba1, cry7Aa1, cry7Ab1, cry7Ab2, cry8Aa1, cry8Ba1, cry8Ca1, cry9Aa1, cry9Aa2, cry9Ba1, cry9Ca1, cry9Da1, cry9Da2, cry9Ea1, cry10Aa1, cry11Aa1, cry11Aa2, cry11Ba1, cry11Bb1, cry11Bb1, cry12Aa1, cry13Aa1, cry14Aa1, cry15Aa1, cry16Aa1, cry17Aa1, cry18Aa1, cry19Aa1, Cry19Ba1, cry20Aa1, cry21Aa1, cry22Aa1, cry24Aa1, cry25Aa1, cry26Aa1, cry28Aa1, cyt1Aa1, cyt1Aa2, cyt1Aa3, cyt1Aa4, cyt1Ab1, cyt1Ba1, cyt2Aa1, cyt2Ba1, cyt2Ba2, cyt2Ba3, cyt2Ba4, cyt2Ba5, cyt2Ba6, cyt2Bb1, 40 kDa, cryC35, cryTDK, cryC53, vip1A, vip2A, vip3A(a), vip3A(b), and p21med. Any of these can be made, modified or developed according to the methods herein.

Other candidate parental nucleic acids relevant to pest resistance include protease and α or β-amylase inhibitors, cholesterol oxidases, polyphenol oxidases, insecticidal proteases, vegitative insecticidal proteins, pathways for polyketides, natural products from microorganisms, fungi, plants, etc., baculoviruses, and the like. A variety of assays for screening modified chimeric nucleic acids are suitable for use in connection with the present invention, including bioassays (e.g., whole organism and cell-based assays), high throughput assays, ATPase release assays, cell morphology assays, alamar blue assays, ³H incorporation assays, trypan blue cell viability tests, competitive binding assays, receptor binding assays, phage display of insect resistance proteins, and many others are described, e.g., in the WO 99/57128 publication. A variety of activities (increased target range, decreased susceptibility to development of resistance by pests, increased potency, increased expression level, etc.) can be monitored. As with herbicide resistance genes noted above, chimeric insect resistance genes made according to the methods herein can be cloned, transduced into plants or other organisms (e.g., to create insect resistant plants or other organisms), and the like. Any activity of interest can be produced according to the methods described herein.

Mycotoxin Detoxification

Other target proteins/nucleic acids/pathways that are suitable for use in the present invention include those that are relevant to mycotoxin detoxification as described, for example, in WO 00/20573. Exemplar targets for mycotoxin detoxification activity include, e.g., enzymes that modify mycotoxins, including monooxygenase such as p450s. P450s are a superfamily of enzymes capable of catalyzing a wide variety of reactions including epoxidation, hydroxylation, O-dealkylations, desaturation etc. One particularly preferred source of p450 parental nucleic acids is the cyp 1, 2 and 3 families of genes, e.g., from humans. Other suitable nucleic acids include those that encode structurally and functionally similar peroxidases and chlorperoxidases, as well as structurally unrelated iron-sulfur methane monooygenases, trichothecene-3-O-acetyltransferase, 3-O-Methyltransferase, glutathione S-transferase, epoxide hydrolases, isomerases, macrolide-O-acytyltransferases, 3-O-acytyltransferases, and cis-diol producing monooxygenases for furan, as well as for non-monooxygenase genes which can catalyze detoxification reactions such as epoxidations, hydroxylations, O-dealkylations, desaturations, etc. can also be used as substrates according to the present invention. Methods for screening for mycotoxin detoxification relevant activities can be screened for using methods such as those described in WO 00/20573. Mycotoxin detoxification relevant activities include, e.g., inactivation or modification of a polyketide, an aflatoxin, inactivation or modification of a sterigmatocystin, inactivation or modification of a trichothecene, inactivation or modification of a fumonisin, an increased ability to chemically modify a mycotoxin, an increase in the range of mycotoxin substrates which the distinct or improved nucleic acid operates on, an increased expression level of a polypeptide encoded by the nucleic acid, a decrease in susceptibility of a polypeptide encoded by the nucleic acid to protease cleavage, a decrease in susceptibility of a polypeptide encoded by the nucleic acid to high or low pH levels, a decrease in susceptibility of the protein encoded by the nucleic acid to high or low temperatures, and a decrease in toxicity to a host cell of a polypeptide encoded by the selected nucleic acid. Suitable screening assays include those that detect, for example, changes (e.g., oxidation, thiol attack, epoxidation) in properties of targets for detoxification (e.g., by physical detection means), oxidation in yeast, selection of cells in the presence of a mycotoxin, pathogen resistance in food products expressing modified mycotoxin detoxification nucleic acids, detection of demethylation (e.g., using scintillating polymeric beads), etc.

Improved Plant Phenotypes

Other parental nucleic acids that are suitable for use in the practice of the present invention include those that encode metabolic enzymes from plants and/or photosynthetic microbes and/or bacteria, including, for example, those described in WO 00/28018 “Modified ADP-Glucose Pyrophosphorylase for Improvement and Optimization of Plant Phenotypes.” Metabolic genes that are suitable for use as parental nucleic acids include ADP-glucose pyrrophosphorylase (ADGPP), ribulose 1,5-bisphosphate carboxylase/oxygenase (RUBISCO) and other genes encoding Calvin cycle enzymes or Krebs cycle enzymes, phosphoenolpyruvate (PEP) carboxylase genes, or the like. For ADGPP, genes encoding both catalytic subunits (small subunit, S; gene designation, S) and allosteric regulatory subunit (large subunit, L; gene designation, L), as appropriate for plant and algal (S₂L₂), as well as bacterial (S₄), can be recombined, selected or otherwise modified or developed according to the methods described herein.

RUBISCO genes suitable for use in the present invention as parental nucleic acids include those described in “Modified Ribulose 1,5-Bisphosphate Carboxylase/Oxygenase,” WO 00/28008. In brief, Rubisco exists in at least two forms: form I rubisco is found in proteobacteria, cyanobacteria, and plastids, e.g., as an octo-dimer composed of eight large subunits, and eight small subunits; form II rubisco is a dimeric form of the enzyme, e.g., as found in proteobacteria. Form I rubisco is encoded by two genes (rbcL and rbcS,) while form II rubisco has clear similarities to the large subunit of form I rubisco, and is encoded by a single gene, also called rbcL. Thus, the method is broadly applicable to evolving biosynthetic enzymes having desired properties, e.g., RUBISCO, including both regulatory subunit (small subunit, S; gene designation, rbcS) and catalytic subunit (large subunit, L; gene designation, rbcL), respectively, as appropriate for Form I (L₈S₈) and Form II (L₂) Rubisco. Nucleic acids encoding either form of RUBISCO can be modified according to the present invention and screened for activity as taught herein or, e.g., in WO 00/28008. For example, a bacterial single subunit Rubisco gene, such as that from Rhodospirillum rubrum (Falcone et al. (1993) J. Bacteriol. 175: 5066), or a fragment thereof, is obtained as a polynucleotide (isolated, synthesized, etc.) and used in the methods of the present invention (e.g., as single-stranded templates or as fragments bound to such templates). Example photosynthetic bacterial sources for the rbcL gene(s) include those from Rhodobacter shaeroides, Rhodospirrilum rubrum and the like. Example photsynthetic dinoflagellate sources for rbcL genes include those from Gonyaulax polyedra (Morse et al. (1995) Science 263: 1522), Amphidinium carterae (Whitney et al. (1998) Aust. J. Plant Physiol. 25: 131), and Symbiodinium (Rowan et al. (1996) Plant Cell 8: 539). A preferred host cell is a strain of photosynthetic bacterium that is transformable and which can be complemented to photoheterotrophic growth by expression of a functional rbcL gene. Phenotype selection of modified genes is performed, e.g., by biochemical assays for RuBP carboxylase and/or RuBP oxygenase activity, or other suitable assay methods. Example photosynthetic bacteria for the rbcL gene(s) include Rhodobacter sphaeroides (Falcone et al. (1998) J. Bact. 170: 5), Rhodospirrilum rubrum (Falcone and Tabita (1993) J. Bact. 175: 5066; Falcone et al. (1991) J. Bact. 173: 2099) and the like. Example cyanobacteria that can serve as a source of rbcL genes include Synechococcus, Cocochloris peniocystis, and Aphanizomenon flos-aquae. Example green algae that can serve as sources of rbcL genes include Euglena gracilis, Chlamadomonas reinhardii, and Anacystis nidulans. Any of these can be made, modified or developed according to the methods herein.

Similarly, further details regarding PEP targets and selection methods are described in “Modifed Phosphoenoylpyruvate Carboxylase for Improvement and Optimization of Plant Phenotypes,” WO 00/28017. For example, Phosphoenolpyruvate (PEP) carboxylase (PEPC; EC 4.1.1.31) is a key enzyme of photosynthesis in those plant species exhibiting the C4 or CAM pathway for CO₂ fixation. The principal substrate of PEPC is the free form of PEP. PEPC catalyzes the conversion of PEP and bicarbonate to oxalacetic acid inorganic phosphate (Pi). This reaction is the first step of a metabolic route known as the C4 dicarboxylic acid pathway, which minimizes losses of energy produced by photorespiration. PEPC is present in plants, algae, cyanobacteria, and bacteria; the enzymatic properties differ based on the source. Nucleic acids encoding PEPC can be modified according to the present invention and screened for activity as taught herein or, e.g., in WO 00/28107.

Lipid Production Genes

Other suitable targets for modification according to the present invention include lipid production genes. Many such suitable genes, pathways and associated screens are described in PCT/US00/09285 “Modified Lipid Production.” A variety of lipid biosynthetic activities can be selected, separately or in combination, including: modulation of lipid saturation for one or more selected lipids produced by a lipid synthetic pathway comprising activity encoded by the one or more selected chimeric lipid biosynthetic nucleic acids, modulation of fatty acid composition in a transgenic plant, algae, animal, bacteria, fungus or other organism expressing the selected chimeric lipid biosynthetic nucleic acid, modulation of fatty alcohol composition in a transgenic plant, algae, animal, bacteria, fungus or other organism expressing the selected chimeric lipid biosynthetic nucleic acid, modulation of a wax composition in a transgenic plant, algae, animal, bacteria, fungus or other organism expressing the selected chimeric lipid biosynthetic nucleic acid, modification of acyl chain length in a lipid produced by a lipid synthetic pathway comprising activity encoded by the selected chimeric lipid biosynthetic nucleic acid, location of fatty acid accumulation in a transgenic plant, algae, animal, bacteria fungus or other organism expressing the selected chimeric lipid biosynthetic nucleic acid, modulation of lipid yield of a transgenic plant, algae, animal, bacteria, fungus or other organism expressing the selected chimeric lipid biosynthetic nucleic acid, an increased ability of a molecule encoded by the selected chimeric lipid biosynthetic nucleic acid, or a cell transduced with the selected chimeric lipid biosynthetic nucleic acid, to chemically modify a lipid or lipid precursor, an increase or alteration in the range of lipid substrates for a cell transduced with the selected chimeric lipid biosynthetic nucleic acid, an increased expression level of a lipid biosynthetic polypeptide in a cell transduced with the selected chimeric lipid biosynthetic nucleic acid, a decrease in susceptibility of a lipid biosynthetic polypeptide in a cell transduced with the selected chimeric lipid biosynthetic nucleic acid to protease cleavage, a decrease in susceptibility of a lipid biosynthetic polypeptide encoded by the selected chimeric lipid biosynthetic nucleic acid in a cell to high or low pH levels, a decrease in susceptibility of a protein encoded by the selected chimeric lipid biosynthetic nucleic acid in a cell to high or low temperatures, and a decrease in toxicity to a cell by a lipid biosynthetic polypeptide encoded by the selected chimeric lipid biosynthetic nucleic acid, as compared to one of the parental nucleic acids, when expressed in a cell.

The chimeric lipid biosynthetic nucleic acid is selected e.g., by detecting one or more of: a change in a physical property of one or more lipid, fatty acid, wax or oil in the presence of a polypeptide or RNA encoded by the selected chimeric lipid biosynthetic nucleic acid, a protein-protein interaction in a two hybrid assay, expression of a reporter gene in a one hybrid assay, growth or survival of a recombinant cell expressing the selected chimeric lipid biosynthetic nucleic acid in an elevated temperature environment, growth or survival of a recombinant cell expressing the selected chimeric lipid biosynthetic nucleic acid in a medium comprising a membrane active compound, relative bioluminescence of a recombinant cell comprising at least one gene from the Lux operon and the selected chimeric lipid biosynthetic nucleic acid, detection of cellular localization of a protein encoded by the selected chimeric lipid biosynthetic nucleic acid, detection of cellular localization of a protein encoded by the selected chimeric lipid biosynthetic nucleic acid to a chloroplast, or endoplasmic reticulum, and detection of cellular localization of a product produced as a result of expression of the selected chimeric lipid biosynthetic nucleic acid in a cell.

A variety of parental nucleic acids are suitable for use in the methods of the invention, including nucleic acids which are the same as, fragments of, or homologous to a nucleic acid encoding a protein such as any of the following: an Acetyl-CoA carboxylase (an ACCase), a homomeric acetyl-CoA carboxylase, a heteromeric acetyl-CoA carboxylase BC subunit, a heteromeric acetyl-CoA carboxylase, a BCCP subunit, a heteromeric acetyl-CoA carboxylase (alpha)-CT subunit, a heteromeric acetyl-CoA carboxylase (beta)-CT subunit, an acyl carrier protein (ACP) (plastidial isoform or mitochondrial isoform), a malonyl-CoA:ACP transacylase, a ketoacyl-ACP synthase (KAS), a KAS I, a KAS II, a KAS III, a ketoacyl-ACP reductase, a 3-hydroxyacyl-ACP, an enoyl-ACP reductase, a stearoyl-ACP desaturase, an acyl-ACP thioesterase (Fat), a FatA, a FatB, a glycerol-3-phosphate acyltransferase, a 1-acyl-sn-glycerol-3-phosphate acyltransferase, a plastidial cytidine-5′-diphosphate-diacylglycerol synthase, a plastidial phosphatidylglycero-phosphate synthase, a plastidial phosphatidylglycerol-3-phosphate phosphatase, a phosphatidylglycerol desaturase (palmitate specific), a plastidial oleate desaturase (fad6), a plastidial linoleate desaturase (fad7/fad8), a plastidial phosphatidic acid phosphatase, a monogalactosyldiacyl-glycerol synthase, a monogalactosyldiacyl-glycerol desaturase (palmitate-specific), a digalactosyldiacyl-glycerol synthase, a sulfolipid biosynthesis protein, a long-chain acyl-CoA synthetase, an ER glycerol-3-phosphate acyltransferase, an ER 1-acyl-sn-glycerol-3-phosphate acyltransferase, an ER phosphatidic acid phosphatase, a diacylglycerol cholinephosphotransferase, an ER oleate desaturase (fad2), an ER linoleate desaturase (fad3), an ER cytidine-5′-diphosphate-diacylglycerol synthase, an ER phosphatidylglycero-phosphate synthase, an ER phosphatidylglycerol-3-phosphate phosphatase, a Phosphatidylinositol synthase, a diacylglycerol kinase, a cholinephosphate cytidylyltransferase, a phosphatidylcholine transfer protein, a choline kinase, a Lipase, a phospholipase C, a phospholipase D, a phosphatidylserine decarboxylase, a phosphatidylinositol-3-kinase, a ketoacyl-CoA synthase (KCS), a (beta)-keto-acyl reductase, and a transcription factor such as CER 2 controlling lipid biosynthetic activity, a fatty acid isomerase, a fatty acid hydroxylase, a fatty acid epoxidase, a fatty acid acetylenase, a methyl transferase related enzyme which alters lipids, (e.g., cyclopropane fatty acid synthases, meromycolic acid synthases, cyclopropane mycolic acid synthases), a diacylglycerol acyltransferases (DGAT), an acyl CO-A reductases, a wax synthase, a Cholesterol:Acyl-CoA acyltransferases (ACAT), and/or a leci then:Acyl-CoA Acyltransferases (LCAT).

For example, in one aspect, one or more of the parental nucleic acids which are used in the methods herein are the same as, or homologous to, a nucleic acid encoding a protein which affects oil yield, such as an ACCase, an sn-2 acyltransferase, an acyltransferase other than sn-2 acyltransferase, a malonyl-CoA:ACP transacylase, an oleosin, a fatty acid binding protein, an Acyl-CoA synthase, or an acyl-ACP synthase. Similarly, at least one of the parental nucleic acids can be the same as, or homologous to, a nucleic acid encoding a protein which affects fatty acid acyl chain length or composition, such as a thioseterase or an elongase. Again, similarly, at least one of the parental nucleic acids can be the same as, or homologous to, a nucleic acid encoding a protein which affects fatty acid saturation, such as a desaturase, a cis-trans isomerase, or a lipoxygenase (LOX). The parental nucleic acids can also be the same as, or homologous to, a nucleic acid encoding a protein which affects fatty acid branch structures, such as a reductase, or to a nucleic acid encoding a protein which affects flavor, such as a Lox protein, a desaturase, a beta-oxidation enzyme, or a hydroperoxide lyase. The parental nucleic acid can be the same as, or homologous to, a nucleic acid encoding a protein which affects polyunsaturation, such as a protein in the polyketide synthase-like operon, a desaturase, or an elongase. The parental nucleic acid can be the same as, or homologous to, a nucleic acid encoding a lipase or a DNA binding protein.

Starch Metabolizing Enzymes

In another aspect, the present invention relates to the modification of starch metabolizing enzymes, to produce novel starch metabolizing enzymes. Candidate starch metabolizing enzyme-encoding parental nucleic acids and assays to screen for novel starch metabolizing enzymes are described in detail in PCT/US00/09840 “Modified Starch Metabolism Enzymes and Encoding Genes for Improvement and Optimization of Plant Phenotypes.” In addition, the present invention also provides new starch compositions produced by novel starch metabolizing enzymes made by the methods herein.

Novel starch metabolizing enzyme activities include one or more of the following enzymatic activities: starch synthase (starch synthetase), amylase (alpha or beta type), branching enzyme (BE, BEI, BEIIa, BEIIb, BEIII, and the like), debranching enzyme (isoamylase or pullulanase), starch phosphorylase, or modified activities thereof. Examples of parental nucleic acids that are suitable for use in the practice of the present invention include genes that encode: starch synthase (both soluble isozymes and bound isozymes), branching enzymes, debranching enzymes (isoamylases and pullulanases), amylase (alpha and beta), and starch phosphorylase, with respect to gene sequences that are derived from higher plants. In certain embodiments, gene sequences encoding microbial starch metabolic enzymes such as glycogen synthase (“GS”; glgA gene product), glgC gene product (ADP glucose pyrophosphorylase), phosphoglucomutase (“pgm”), and the like are employed in the invention methods. In certain embodiments, gene sequences encoding animal liver glycogen synthase or yeast glycogen synthase are used.

As with any relevant parental nucleic acid described herein, relevant nucleic acids can be obtained, e.g., by cloning, synthesis, PCR, from deposited materials, or using any other available source or method.

Plant Disease Responses

For example, the invention provides methods for identifying and improving R genes and elicitors involved in plant defense responses. Plant defense responses include plant disease responses to pathogens, such as viral, bacterial, fungal, insect or nematode pathogens and pests, as well as responses to environmental stresses such as heat, drought, uv irradiation and wounding. One aspect of the present invention relates to methods for identifying plant disease resistance genes (R) with novel characteristics, e.g., novel elicitor interactions, kinase activation and downstream signalling. Embodiments of the invention provide methods of identifying such novel R genes by modifying R genes according to the methods herein to produce a diversified library of R genes, and identifying library members with specified characteristics.

Identification of R genes with characteristics of interest is performed, e.g., by expressing the R gene product in a plant cell, and screening for improved traits, or other desirable outcomes. Expression occurs, e.g., following stable integration of the recombinant R gene operably linked to a functional promoter, or via cytoplasmic expression after introduction of the recombinant R gene via a non-integrating viral vector. Such vectors include both RNA and DNA viruses, e.g., tobamoviruses, petexviruses, potyviruses, tobraviruses, and geminiviruses. In some embodiments expression is regulated by a viral subgenomic promoter. In other embodiments, the recombinant R gene is introduced to the plant via infection with a plant pathogen, such as a bacterial pathogen, that transfers the recombinant R gene, optionally including a target signal, according to pathogen infection mechanisms into the plant cell. Currently, there are more than 20 R genes cloned from different plant species. Many of them are members of large gene families, which provide excellent pools of candididate genes for modification, because members of each gene family usually have relatively high sequence homology as well as ample diversity. A variety of R genes are suitable for use as parental nucleic acids according to the methods described herein, including: Bs2, Cf2, Cf4, Cf9, Hcr2, Hcr9, Xa21, Rp1-D, Rpp5, Rpp8, RPM1, RPS2, RPS4, PRF, L6, M, I2, N, Rx, Mi, Dm3, Xa1, Pib, Pto, Pti1, Mlo, Hs1pro-1, LRK10, Fen, etc. A description of these and other suitable parental nucleic acids, as well as screens and assays, is provided in U.S. Ser. No. 60/202,233.

Other Targets

In addition to the use of genes, gene fragments, pathways etc., as substrates for the diversity generating/screening processes noted herein, other suitable components can also be used as substrates for the reactions. For example, viruses, viral vectors, agrobacterium vectors, plasmids, and genomes are all suitable targets for the methods herein. For example, U.S. Ser. No. 60/167,452 “Shuffling of Agrobacterium and Viral Genes, Plasmids and Genomes for Improved Plant Transformation,” describes a variety of vectors, viruses and the like, all of which can be modified according to the methods herein. For example, targets for the procedures herein include agrobacterium and its components (e.g., the right and left T-DNA borders, which can include engineered features such as PCR primer binding sites and the like. Furthermore, relevant genes (e.g., in the case of agrobacterium, the vir genes (e.g., vir A, vir B, vir C, vir D, vir E, vir G, chvE)) can be modified. Any property relevant to the vector of interest can be selected for. For example, U.S. Ser. No. 60/167,452 describes a variety of properties that can be selected for, including one or more of: insert precision, targeted insertion, improved host range, transformation efficiency, in planta transformation of leaves, in planta transformation of cut stems, in planta transformation in the absence of exogenous phytohormones, transformation without in vitro culture, and chloroplast targeting. A number of other references noted herein provide additional suitable targets for vector/virus recombination, which can be adapted to the present invention.

Industrially-Related Parental Nucleic Acids and Expression Products

Industrially important enzymes such as monooxygenases (e.g., p450s, DBT monooxygenases encoded by the dszC gene from, e.g., Rhodococcus spp., or the like), dioxygenases, lipases, esterases, proteases, glycosidases, glycosyl transferases, phosphatases, kinases, haloperoxidases, lignin peroxidases, diarylpropane peroxidases, epoxide hydrolases, nitrile hydratases, nitrilases, transaminase, amidases, acylases, dehalogenases, isomerases, epimerases, glucose isomerases, amino acid racemases, and nucleases are also generally preferred targets. Proteins which aid in folding such as the chaperonins are preferred targets. Many of these and other industrial enzymes, and corresponding nucleic acid sequences, are provided in various published documents including, e.g., WO 00/01712 “CHEMICALLY MODIFIED PROTEINS WITH A CARBOHYDRATE MOIETY,” WO 00/37658 “CHEMICALLY MODIFIED ENZYMES WITH MULTIPLE CHARGED VARIANTS,” WO 00/28007 “CHEMICALLY MODIFIED MUTANT SERINE HYDROLASES SHOW IMPROVED CATALYTIC ACTIVITY AND CHIRAL SELECTIVITY,” WO 99/37324 “MODIFED ENZYMES AND THEIR USE FOR PEPTIDE SYNTHESIS,” WO 99/34003 “PROTEASES FROM GRAM POSITIVE ORGANISMS,” WO 99/31959 “ACCELERATED STABILITY TEST,” and WO 98/23732 “CHEMICALLY MODIFIED ENZYMES,” all of which are incorporated herein by reference in their entirety for all purposes. These and additional nucleic acids are present in GENBANK® or other publicly accessible databases.

The following present a series of non-limiting examples of industrial enzymes suitable for improvement by the methods disclosed herein. Accordingly, nucleic acids which correspond to any of the noted proteins can be recombined by the methods herein and selected for new or improved activities.

Proteases

Proteases are enzymes that hydrolyze peptide bonds in proteins. The extent to which a protease acts on a protein is referred to as its degree of hydrolysis (% DH); or simply, the percentage of peptide bonds hydrolyzed. The necessary amount of hydrolysis of a protein varies depending on the end-use. For example, with proteases in detergents the objective is typically to achieve as much hydrolysis of the protein-based stain as possible. On the other hand, in cheese making, the goal may be only to break a single bond in the casein molecule in order to coagulate the milk. Applications for proteases include in, e.g., laundry detergents, cheese making, bating (softening) leather, modifying food ingredients (e.g., soy protein), and flavor development.

The subtilisin family of serine proteases constitute the largest volume and highest value segment of the industrial enzyme industry, due to its use in a wide variety of household and industrial cleaning products. Its improvement has been the subject of, perhaps, more protein engineering and more scientific publications than any other protein. For example, bacterial proteases can be used for improving fermentative yeast growth, in laundry detergents, and many other applications.

Bacillus subtilisin sequences known in the art include those corresponding to subtilisin BPN′ from B. amyloliquefaciens (Vasantha et al., (1984) J. Bacteriol. 159:811-819) subtilisin Carlsberg from B. licheniformis (Jacobs et al., (1985) Nucleic Acids Res. 13:8913-8926), subtilisin DY (Nedkov et al., (1985) Biol. Chem. Hoppe-Seyler 366:421-430), subtilisin amylosacchariticus (Kurihara et al. (1972) J. Biol. Chem. 247:5619-5631), and mesenticopeptidase (Svendsen et al. (1986) FEBS Lett. 196:228-232). See also, Von der Osten et al., (1993) J. Biotechnol. 28:55-68.

Variants of Bacillus subtilisins for use in a wide variety of commercial applications are described in, for example, PCT publications WO 99/20770, WO 99/20769, WO 99/20727, WO 99/20726, WO 98/55634, and WO 95/10615, and many other publications. See also, U.S. Pat. Nos. 5,801,038, 5,763,257, 5,700,676, 5,441,882, 5,346,823, 5,316,941, and 5,310,675.

The sequence of a subtlisin-like protease from a human source is described in PCT Publication No. WO 99/53078. That publication, and WO 99/53038, describe proteases exhibiting reduced allergenicity for a variety of commercial applications such as, e.g., personal care products.

Fungal subtilisins include: proteinase K from Tritirachium albam (Jany et al. (1985) Biol. Chem. Hoppe-Seyler 366:485-492) and thermomycolase from the thermophilic fungus, Malbranchea pulchella (Gaucher et al. (1976) Methods Enzymol. 45:415-433). Additional sequences of subtilisins and subtilisin-like proteases (subtilases) are found in Siezen et al. (1991) Protein Engineering 4: 719-737 and in Siezen & Leunissen (1997) Protein Sci 6:501-523.

Nucleic acid and amino acid sequences of cysteine proteases from Bacillus subtilis are provided in PCT publication No. WO 99/04016. Nucleic acid and amino acid sequences are available for plant cysteine proteases, such as papain (Cohen, L. W. et al (1986) Gene 48:219-227), actinidin (Praekelt, U. M., et al. (1988) Plant Mol. Biol. 10: 193-202 (1988), and bromelain (Muta, E. et al. (1993) GenBank Nucleotide Accession No. D14058).

Sequences of metalloproteases from Bacillus are provided, for example, in PCT publication Nos. WO 99/34003, WO 99/34002, WO 99/34001, WO 99/33960, WO 99/33959, WO 99/14342, and WO 99/14341.

Other protease examples include, savinases, thermitases, subtilisin BLAP from B. licheniformis, mutant/modified subtilisins (see, e.g., U.S. Pat. Nos. 5,972,682 and 5,955,340), serine proteases SP1, SP2, SP3, SP4 and SP5 (see, e.g., WO 99/03984), subtilisin sprC (see, e.g., U.S. Pat. No. 5,677,163), and naturally-occurring or recombinant non-human proteases with altered net charges (see, e.g., WO 99/20771). Accordingly, all of these enzymes can be modified using the methods on the invention.

Amylases—Enzymes that Hydrolyze Starch

Native starch is a polymer made up of glucose molecules linked together to form either a linear polymer called amylose or a branched polymer called amylopectin. In amylose, glucose units are linked by 1-4 bonds. In amylopectin, glucose is also linked by 1-4 bonds but in addition, branch points occur every 20 to 25 glucose units where an additional glucose is linked by 1-6 bonds. Amylases of commercial importance include the following:

Alpha-Amylases

These enzymes rapidly cleave internal 1-4 bonds in an “endo” fashion to yield shorter water-soluble chains called dextrins. Some of these alpha-amylases are more thermostable than others. Certain alpha-amylase enzymes and nucleic acids, such as, the bacillus alpha-amylase genes are described by Gray et al. (1986) J. Bacteriology 166:635-64 and Ihara et al. (1985) J. Biochem. 98:95-103 (B. licheniformis and B. stearothermophilus), and Takkinen et al. (1983) J. Biol. Chem. 258:1007-1013 (B. amyloliquefaciens). Mutant alpha-amylases which are, e.g., oxidatively-stable, or show altered pH and/or altered thermal stability profiles are described in, for example, PCT Publication Nos. WO 99/29876, WO 99/09183, WO 98/26078, WO 96/39528, WO 96/30481, WO 99/02702, WO 96/05295, WO 94/18314, WO 95/35382, WO 96/23873, WO 97/43424, WO 94/02597, WO 94/18314, WO 91/00353, WO 96/30481, WO 96/05295, and WO 94/18314. See also, U.S. Pat. Nos. 6,080,568, 6,008,026, 5,958,739, 5,736,499, 5,849,549, 5,824,532, and 5,763,385. Accordingly, all of these enzymes can be modified using the methods on the invention.

Beta-Amylases

Beta-amylases cleave 1-4 bonds but attack soluble starch in a different manner than alpha-amylases, i.e., they attack in an “exo” fashion. That is, the enzyme splits off maltose (a disaccharide) in a step-by-step manner from one end of the starch polymer.

The nucleic acid and amino acid sequences of beta-amylase genes from two barley cultivars have been reported (Kreis M et al. (1987) Eur. J. Biochem. 169:517; and Yoshigi N. et al (1994) J. Biochem. 115: 47-51). U.S. Pat. No. 5,863,784 describes barley beta-amylase variants showing improved thermostability. The nucleic acid and protein sequences of a beta-amylase from potato in described in PCT publication No. WO 00/08185.

Kitamoto, N., et al (1988; J. Bacteriol. 170: 5848-5854) describe the nucleic acid and protein sequence of a thermophilic beta-amylase from Clostridium thermosulfurogenes. Siggens, K. W. (1987; Mol. Microbiol. 1: 86-91) provides a beta-amylase gene from Bacillus circulans. Kawazu, T., et al (1987; J. Bacteriol. 169: 1564-1570) provide a beta-amylase gene from Bacillus (Paenibacillus) polymyxa.

Fungal Amylases

These are alpha-amylases with a slightly different pattern of action. They are more “aggressive” in the hydrolysis of starch, yielding mostly maltose and some oligomers. They are an alternative to beta-amylases for making maltose syrups. Applications of alpha-amylases include, e.g., in the corn syrup industry for the production of syrups containing up to 60% maltose and in the baking industry for flour improvers. Fungal amylase is also used, e.g., to decrease fermentation time. Genes encoding fungal alpha-amylases are described in, for example, Matsuura et al. (1984) J. Biochem. (Tokyo) 95:697-702 (Taka-amylase A from Aspergillus oryzae) and in Boel et al., (1990) Biochemistry 29:6244-6249 (acid alpha-amylase from A. niger).

Glucoamylases

Glucoamylase or amyloglucosidase is another amylase that catalyzes the hydrolysis of 1-4 linkages in starch. Single molecules of glucose are cleaved in a step-by-step manner from one end of the starch molecule. Glucoamylases can also hydrolyze 1-6 bonds but at a much slower rate than the 1-4 bonds. Applications for these enzymes include, e.g., in the corn syrup industry to break down dextrins in the production of glucose syrups.

PCT publication WO 00/04136 describes the Aspergillus niger GI glucoamylase gene (AMG, Novo-Nordisk) and variants having improved thermal stability and/or increased specific activity.

Hata, Y., et al (1991; Agric. Biol. Chem. 55:941-949) provide glucoamylase cDNA from Aspergillus oryzae. Dohmen, J. R., et al., (1990; Gene 95, 111-121) provide a Schwanniomyces (Debaryomyces) occidentalis glucoamylase gene

Pullulanases

This debranching enzyme hydrolyzes the 1-6 bonds in amylopectin molecules thus eliminating the 1-6 branch “barriers.” For example, a beta-amylase cannot bypass a branched 1-6 linkage to attack linear 1-4 bonds on the other side. However, with a debranching enzyme such as pullulanase, beta-amylase can be used to convert a starch slurry into a syrup with high amounts of maltose. They can also be used with glucoamylase in the saccharification of dextrins to glucose in the corn syrup industry.

WO 98/50562 describes a pullulanase gene from corn, and protein sequences of related plant pullulanases from Oryza sativa and Spinacia oleracea. Genes and/or protein sequences corresponding to pullulanases from Bacillus deramificans, B. naganoensis, B. acidopullulyticus, and B. sectorramus are described in U.S. Pat. No. 5,721,127, U.S. Pat. No. 5,055,403, U.S. Pat. No. 4,560,651, and U.S. Pat. No. 4,902,622, respectively. WO 99/45124 provides the sequences a number of pullulanases from microbial sources, such as B. subtilis and Klebsiella pneumonia, and sequences of modified pullulanases. Other pullulanase examples include those described in, e.g., PCT publication Nos. and WO 99/45124, and U.S. Pat. Nos. 6,074,854, 5,817,498, 5,736,375, 5,721,128, and 5,721,127. Accordingly, all of these enzymes can be modified using the methods on the invention.

Cellulases

Many different enzymes are needed to totally hydrolyze fibre. For example, endocellulases are capable of hydrolyzing the 1-4 bonds randomly along the cellulose chain. Exocellulases cleave off glucose molecules from one end of the cellulose strand. Cellulases and cellobiases are often used in conjunction to transform complex cellulose-containing raw materials into glucose.

Cellulases produced in microorganisms may comprised several different enzyme classes, including cellobiohydrolases (“CBH”), endoglucanases (“EG”), and beta-glucosidases (“BG”) (Wood et al. (1988) Meth. Enzymol. 160, 234). The classifications of CBH, EG and BG can be further expanded to include multiple components within each classification. Various bacteria and fungi contain multiple CBHs and EGs; for example, the filamentous fungus Trichoderma reesei contains 2 CBHs (denoted CBH I and CBH II), and at least 3 EGs (denoted EG I, EG II, and EG III).

Endoglucanases for obtaining a “stonewashed” look in colored fabric are described in U.S. Pat. No. 5,650,322. Sheppard et al. (1994; Gene 150:163-167) provides the DNA and amino acid sequence of a Fusarium oxysporum C-family endoglucanase. PCT publication WO 91/17244 describes the DNA and amino acid sequence of a Humicola insolens endoglucanase 1 (EGI). FIG. 1 of U.S. Pat. No. 5,912,157 provides an alignment of the amino acid sequences of three endoglucanases and one cellobiohydrolase: Fusarium oxysporum endoglucanase EG1 (EG1-F); Humicola insolens endoglucanase EGI (EG1-H); Trichoderma reesei endoglucanase EG1 (EGI-T); and Trichoderma reesei cellobiohydrolase.

Sequences of EGIII and EGIII-like cellulases and variants thereof are provided in PCT publications WO 00/37614 and WO 99/31255 (from Trichoderma reesei and other sources)(see also, U.S. Pat. No. 5,770,104), and WO 94/21801 (from Trichoderma longibrachiatum) (see also, U.S. Pat. No. 5,475,101). Variant EGIII cellulases with altered properties are also described in WO 00/14208 and WO 00/14206.

Beta-glucosidases from Trichoderma reesei are described in U.S. Pat. No. 6,022,725. Beta-glucosidases are also described in, e.g., U.S. Pat. No. 5,997,913.

Combinations of fungal CBH I type components and EG type components are described in U.S. Pat. Nos. 5,668,009 and 5,654,193. Multmeric cellulases are also described in PCT publication WO 98/28411 and U.S. Pat. No. 5,989,899.

Various Bacillus cellulases are described in PCT publications WO 97/34005 (see also, U.S. Pat. No. 6,063,611) and WO 96/34108 (see also, U.S. Pat. No. 5,586,165). U.S. Pat. No. 6,074,867 describes the DNA and amino acid sequence of an endoglucanase from a thermophilic archaeal bacteria.

Other cellulase examples include actinomycetes-derived cellulases (see, e.g., WO 00/09707, WO 99/25847, and WO 99/25846), cellulases from Trichoderma longibrachiatum (see, e.g., PCT publication No. WO 98/15619 and U.S. Pat. Nos. 6,017,870, 5,874,276, and 5,753,484), cellulase mutants including ES cellulase (see, e.g., PCT publication Nos. WO 99/10481 and WO 98/13465, and U.S. Pat. No. 5,871,550), WO 99/29821, WO 00/34565, WO 00/09707, WO 99/25847, and WO 99/25846. Accordingly, all of these enzymes can be modified using the methods on the invention.

Hemicellulases

Hemicelluloses may be made up of 5 or 6 different sugar components. By comparison, cellulose and other beta-glucans have only glucose molecules. Many have branched structures while cellulose does not. Hemicelluloses are usually named according to the predominant sugar making up the main chain. Hence they are referred to as xylans, mannans, glucomannans and galactoglucomannans. There are a corresponding variety of hemicellulases capable of degrading them, some of which are described below.

Xylanases are frequently used paper pulp bleaching/delignification, reducing the need for chlorine and/or peroxide-containing chemicals in the pulp bleaching process, and for—treating feed compositions. Xylanases from various sources are described in, e.g., U.S. Pat. Nos., 5,902,581, 5,683,911, and 5,437,992, and PCT publication Nos. WO 95/29998 and WO 97/20920.

Sequences of xylanases from fungal sources are described in WO 92/17573 (Humicola insolens); WO 92/01793 (Aspergillus tubigensis); WO 91/19782 and EP 463 706 (Aspergillus niger).

Mannanases from Bacillus amyloliquefaciens are described in WO 97/11164. Accordingly, all of these enzymes can be modified using the methods on the invention.

Pectinases

Pectins differ from other common carbohydrates because the main component is not a simple sugar, but a sugar acid, i.e., galacturonic acid. Commercial pectinase preparations usually contain a complex of enzymes including endo- and exopectinases, pectinesterases and pectin lyases. Applications include, e.g., extraction of fruit juice, de-pectinization of fruit juice, winemaking, and cotton scouring.

WO 99/27083 and WO 99/27084 describe the sequences of pectate lyases, pectin lyases, and polygalacturonases (collectively known as “pectinases”) from Bacillus licheniformis. Pectate lyases from a wide variety of microbial and plant sources have been described, including Bacillus subtilis (Nasser et al. (1993) FEBS Lett. 335:319-326), Bacillus sp. YA-14 (Kim et al. (1994) Biosci. Biotech. Biochem. 58:947-949). Two pectin lyase genes, pelA and pelB, have been cloned from Aspergillus niger (Kusters-van Someren, M., et al. (1991) Curr. Genet. 20:293-299, and Kusters-van Someren, M., et al. (1992) Mol. Gen. Genet. 234:113-120). Accordingly, all of these enzymes can be modified using the methods on the invention.

Isomerases

Isomerases are a class of enzymes that catalyze isomer conversion reactions. One of these reactions that is carried out industrially is the conversion of glucose to fructose. This is one of the key enzyme reactions in the high fructose corn syrup industry. Isomerization is usually carried out, e.g., in large packed-bed reactors. Some of the columns contain up to 3.5 metric tons of enzyme.

Glucose isomerases are described in WO 90/00601 and in U.S. Pat. Nos. 5,916,789, 5,900,364, and 5,811,280. WO 00/27215 describes the use of glucose isomerases in baking and describes sequences suitable for this purpose. Plant xylose isomerases are described in WO 96/24667. Disulfide bond isomerases are described in, e.g., PCT Publication No. WO 99/04019. Accordingly, all of these enzymes can be modified using the methods on the invention.

Lipases

Lipases act on triglycerides. Sometimes a particular lipase will act on specific types of fatty acids within the triglyceride structure. One of the best-known applications is the removal of fatty stains from laundry. Other applications include, e.g., the de-greasing of hides, in flour improvers, the development of cheese flavours, and pitch removal in paper mills.

WO 92/05249, WO 94/25577, WO 95/22615, WO 97/04079, WO 97/07202 and WO 99/42566 disclose the sequences of wild-type Humicola lanuginosa lipase (Lipolase®, Novo-Nordisk) and variants thereof. WO 98/45453 describes a lipase from Aspergillus tubigensis and its variants. WO 98/08939, WO 95/35381, and WO9530744 provide sequences of various Pseudomonas lipases and variants having altered properties. See also, U.S. Pat. No. 6,017,866.

Cutinases and lipases from Fusarium solanii are described in U.S. Pat. No. 5,990,069. Variants of fungal cutinases having altered properties are described in WO 00/34450. See also, U.S. Pat. Nos. 5,512,203 and 5,389,536. Accordingly, all of these enzymes can be modified using the methods on the invention.

Oxidoreductases

Oxidoreductases are a major class of enzymes existing in nature. As the general name indicates, these catalyze chemical reductions and oxidations and are involved in the breakdown and synthesis of many biochemicals. They account for approximately one quarter of all known enzymes. Some examples which can be modified according to the methods of the invention are described below.

Glucose oxidase catalyzes the conversion of glucose to gluconic acid. One major use of the enzyme is to prevent undesirable Maillard browning reactions, which can affect food color and flavor. Another application involves the use of glucose oxidase as an oxygen scavenger, which can be used to prevent off-flavors in juices. It also helps to preserve color and to maintain the stability of sensitive food ingredients, e.g., ascorbic acid.

Catalases catalyze the decomposition of hydrogen peroxide, which is converted into oxygen and water and are used, e.g., in bleach cleanup in the textile industry. Cotton is normally bleached with hydrogen peroxide before dyeing and this can be neutralized easily with catalase. Catalase is also used to neutralize hydrogen peroxide after it has been used to disinfect contact lenses.

Glucose oxidases are described in PCT publication WO 97/24454 and U.S. Pat. Nos. 5,783,414 and 5,998,179. Catalases from, e.g., Aspergillus niger are described in U.S. Pat. No. 5,360,901 and PCT publications WO 93/18166 and WO 93/17721. Sequences of laccases from a variety of microbial sources, and variants having altered properties, are described in PCT publications WO 98/55628, WO 98/27198, WO 98/38286, and WO 98/38287. See also, U.S. Pat. No. 5,980,579 and PCT publication Nos. WO 98/27264 and WO/98/13474.

Glycosidase

Various glycosidases including, endo-D, endo-H, endo-F, PNGaseF (or endo-beta-N-acetylglucosamimidase, endo-alpha-N-acetylgalactosamimidase or endo-beta-N-galactosidase) are described in, e.g., U.S. Pat. Nos. 5,356,803 and 5,258,304. Accordingly, all of these enzymes can be modified using the methods on the invention.

Laccase

Laccase, which oxidizes certain dyes, is also known as polyphenol oxidase. A laccase transfers electrons from dye precursors to oxygen in the air. This produces dye radicals that react with each other to dye, e.g., hair. Laccases can be modified using the methods on the invention.

Secretion Factors

Secretion factors, e.g., for increasing the secretion of proteins from gram-positive microorganisms, such as secretion factors SecDF and SecG from Bacillus

Other Enzymes

Alpha beta hydrolase-fold enzymes are described in, e.g., WO 99/27081, while isatin hydrolases are described in, e.g., WO 97/19175. Mannanases, such as those form Bacillus amyloliquefaciens are described in, e.g., PCT publication No. WO 97/11164. Accordingly, these enzymes can also be modified using the methods on the invention.

### INDUSTRIAL APPLICATIONS

The following present a series of non-limiting examples of industrial enzyme applications and the nature of the kinds of properties which such applications involve. Many of the enzymes are also described above. In nearly all ensuing applications, development of enzymes with a combination of inexpensive production methodologies, high activity under defined operational conditions and long term storage and process stability are suitable improvement targets for the methods of the invention. In many cases the cost-limiting performance attribute will be enzyme lifetime (total turnover) under process conditions. The relevant enzymes or other proteins can be modified according to the methods herein and selected for activities relevant to any of those noted below.

Distillation

Starch Liquefaction

Before enzymes can attack starch, it must be gelatinized. Traditionally, this is done by pressure cooking. Potatoes, for example, are heated to 150° C. at a pressure of five atmospheres. Upon sudden release of pressure, the cell walls of the potatoes explode, releasing the starch. In this case, the enzymes are added to the mash after cooking, but in other cases a highly heat-stable enzyme can be used in the cooker itself. Recently, the older, non-pressure cooking method has been gaining popularity in smaller distilleries. Instead of temperatures around 150° C., the maximum temperature is from 60° C. to 95° C. There are obvious energy savings and there is no need to invest in pressure vessels. In either processing technique, alpha-amylases are used to break down the gelatinized starch into short molecular fragments (dextrins).

One target for the improvement of enzymes for this process, e.g., according to the present invention, include the development of hyperthermostable cell wall degrading enzymes (cellulases, pectinases and glycosidases) and alpha amylases capable of functioning at or above 90° C., and preferably above 100° C. in the presence of potatoes and slightly elevated pressures. Thus, appropriate enzymes as noted above are developed according to the methods of the invention and screened for these activities.

Starch Saccharification

Following liquefaction, the second step in a typical distillery operation is saccharification. In this step, an amyloglucosidase is used to degrade the starch molecules and the dextrins. If left for sufficient time, these enzymes are capable of achieving the complete degradation of starch into fermentable sugars (e.g., glucose). Low activity of currently available amyloglucosidases, cellulases and other polysaccahride-degrading and debranching enzymes limit the practicality of single step saccharification and fermentation for both the production of spirits and fuel alcohol. By screening enzymes, recombined using the methods disclosed herein, of these classes for a combination of beneficial properties (such as efficient expression in a heterologous host and elevated forward rate kinetics under fermentor-like conditions yields enzyme with improved ability to liberate fermentable sugars from insoluble or otherwise intractable biopolysaccharide.

In one example, host cells containing recombined amyloglucosidase and dextrinase genes can be plated and picked into microwell cultures each containing 20 colonies of transformed bacteria from the resulting library. Each of these minicultures (200 μl in 96 well microtiter plates) is allowed to grow for 8-48 hours in media containing only starch and dextrin as sole carbohydrate sources. The optical densities at 600 nm can be measured every hour and plotted. Wells exhibiting increased opacity within the first 48 hours are scored and the fastest growing cultures are deconvoluted either by serial dilution strategies or by repacking parental clones from copies of the parental plates.

Clones preliminarily identified as positive for enhanced growth can be reexamined at the 24 well level and then in micro chemostats containing 1-10 ml medium. Those clones remaining positive for enhanced growth on the selected carbon sources can be identified as positive and subjected either to additional rounds of mutagenesis, recombination, template-directed recombination (with one another) or other forms of protein improvement. Accordingly, appropriate enzymes can be modified using the methods on the invention and screened for these activities.

Aiding Fermentation

Enzymes can also be used as processing aids. For example, starch-containing cereals, such as corn, tend to be low in soluble nitrogen compounds. This results in poor yeast growth and increased fermentation time. The addition of proteases releases nitrogen from the cereal proteins, thus supplying the yeast's nitrogen requirement. Accordingly, appropriate enzymes can be modified using the methods on the invention and screened for activities, e.g., which aide fermentation.

Fuel Alcohol

Ethanol produced from excess cereal and bio-mass production may represent an important source of fuel extenders or octane boosters. Some carbohydrate raw materials (sugar cane extract or molasses, for example) can be fermented without further treatment. However, this is not true for starch-based raw materials which are at least partially processed into fermentable sugars.

Though the equipment is different, the principles for using enzymes to aid in production of fuel alcohol from starch are the same as for producing alcoholic beverages. Classes of enzymes, whose improvement according to the methods of the invention, will help decrease the cost and complexity of distiller and fuel alcohol production include the following:

Bacterial Amylase

Bacterial amylase is typically used for liquefaction of mashes containing starch at mid-range temperatures. Screening of improved bacterial amylases is done by creating microwell arrays containing simulated or actual mash from a starch containing biological material, such as potatoes. Space-time yield of glucose and short-chain glucose oligomers is done by rapid glucose detection using either glucose sensitive electrodes or rapid colorimetric methods under standard reaction conditions. In a simple form of the test glucose monitoring devices such as blood glucose analyzers are used. Additional performance requirements can be incorporated into the same or a separate screen such as by measuring appearance of sugar monomers and/or oligomers in the presence of elevated an elevated temperature. Clones exhibiting increased rates at process-optimal temperatures (e.g., 60° C.<T<90° C.) are identified, optionally sequenced, and recursively mutagenized using template recombination, recombination, stochastic and nonstochastic mutagenesis methods.

Alternative bacterial alpha amylases can be used for high temperature liquefaction of starch containing mashes (e.g. Novo Nordisk's Liquozyme®, Termamyl).

Dextrinases

Dextrinases can be used to break down dextrins completely to fermentable sugars. Dextrins represent a diverse family of cyclic and linear glucose containing polymers and oligomers. To enhance the breadth of present dextrinases via the present invention, clones can be obtained, converted to single-stranded versions of one strand and single stranded fragments of the other, followed by fragment extension, ligation, parental strand elimination, second strand synthesis, ligation and transformation into a suitable expression construct and host.

Transformants can be identified by, e.g., selection on agar plates containing 50 μg/ml ampicillin. Transformants can be re-gridded onto master plates, pooled into micro-wells containing growth media, grown to saturation. To each well is added 1/10th volume of 1% Triton X-100 and 10 mM polymixin B as permeabilizing agents. Ten μl each of these suspensions are added in parallel to corresponding wells on microtiter plates containing pH 7.4 buffered solutions each plate with a different commercially purchased or synthesized linear or cyclic dextrin. Incubation of each plate at room temperature for 4 hours is followed by glucose detection as described herein. Individual wells are characterized by both the magnitude and breadth of their dextrinase activity. Those exhibiting elevated activity along both dimensions are selected for further characterization and improvement, if necessary. Subsequent rounds of mutagenesis and/or recombination and screening can be conducted as described herein.

Animal Feed

Enzymes are added to feed either directly or as a pre-mix along with vitamins, minerals, and other feed additives. Enzyme products for animal feed are now available to degrade substances such as phytate, glucan, starch, protein, pectin-like polysaccharides, xylan, raffinose, stachyose, hemicellulose and cellulose. All of these can be improved by the methods described herein for specific animal digestive tracts and specific feed materials. In particular, there is a need for a “scaffold set” of proteins with which most feeds can be treated and from which improved derivatives can be easily developed. The main benefits of supplementing feed with enzymes, as revealed by the many feed trials carried out to date, are faster growth of the animal, better feed utilization (feed conversion ratio), more uniform production, and, e.g., an improved environment for birds, e.g., due to reductions in “sticky droppings” from chickens. Enzymes, in this area, that can be improved by the methods described herein include the following:

Phytases

Approximately 50-80% of the total phosphorus in pig and poultry diets is present in the form of phytate (also known as phytic acid). The phytate-bound phosphorus is largely unavailable to monogastric animals, as they do not naturally have the enzyme needed to break it down, i.e., phytase. Phytase in the diet helps to reduce the environmental impact of phosphorus from animal manure in areas with intensive livestock production and to release bound phosphorus other essential nutrients to give the feed a higher nutritional value.

Polysaccharide-Degrading (Non-Starch) Enzymes

Much of the energy in cereals, such as wheat, barley, and rye remains unavailable to monogastrics such as pigs and poultry due to the presence of non-starch polysaccharides (NSP) which interfere with digestion. This prevents access of the animal's own digestive enzymes to the nutrients contained in the cereals. Also, NSP can become solubilized in the gut and increase gut viscosity, resulting in digestive complications, including loss of other nutrients. Carbohydrases which aid in the break down of NSP, help to release energy and nutrients from the gut contents. This results in improved feed utilization, especially in monogastric animals.

In addition, multi-component feed additives may have several of the following, any of which can be improved by the methods described herein, depending on the diet of the livestock.

Beta Glucanases

Beta glucanases and related multi-component enzymes are used in poultry and pig feeds to aid in digestion of high barley diets. Note, they often contains alpha glucanase activity as well.

Alpha Glucanases

Alpha glucanases are generally dual component enzymes containing alpha-amylase and beta-glucanase activities for use in high barley. It would be desirable to rebalance the alpha and beta activities of the enzymes to match the ideal feeds that exist here. Accordingly, one aspect of the present invention includes the application of the methods herein to Alpha glucanase modification to provide this rebalancing.

Digestive Proteases

Digestive proteases (e.g. trypsin, pepsin, or the like) are used to improve the digestibility (and nutritional capture) of feed proteins. Accordingly, these enzymes can be modified according to the present invention, including selection for improved digestibility and and nutritional capture) of feed proteins.

Endoxylanases

Endoxylanase is used to enhance polysaccharide digestion and utilization in poultry and pig feeds wherein the major (or only) cereal ingredient is wheat. Accordingly, this enzyme is modified according to the methods herein to enhance polysaccharide digestion and utilization in poultry and pig feeds in these applications.

Baking

Amylogluosidase

Amylogluosidase is added to certain doughs to increase the release of glucose, which is advantageous for quick-recovery of doughs that will be chilled or frozen. It also improves resulting crust color. Accordingly, these enzymes can be modified using the methods on the invention.

Fungal Alpha Amylases

Fungal alpha amylases are used to assure reliable rising properties doughs containing wheat flour, such as for used in bread production. Accordingly, these enzymes can be modified using the methods on the invention.

Fungal Amylases

Fungal amylases may be combined with pentosanase to treat either high-wheat or other flours to assure reliable rising properties (timing and volume). Typically, both are of a fungal origin. All of these enzymes can be modified using the methods described herein.

Glucose Oxidase

Glucose oxidase is used to improve of dough stability and can be developed according to the methods disclosed herein.

Neutral Protease

Neutral protease can be used to degrade proteins in flour such as for making biscuits, crackers, and cookies (e.g., controls swelling or rising properties). Accordingly, these enzymes can be modified using the methods on the invention and screened, e.g., for these properties.

Maltogenic Amylase

Maltogenic amylase (usually bacterial in origin) is used for antistaling. Accordingly, these enzymes can be modified using the methods described herein and selected for these properties.

Lipase

Purified or semi-purified 1,3-specific lipase is used to control the lipid content and structure in certain baking operation. It is desirable to develop lipases, according to the methods of the invention, with the appropriate selectivity, e.g., which can be used in a less pure form without resulting in contamination with unwanted hydrolase activities.

Pentosanases

Pentosanases are xylanases/hemicellulases used for improving both dough handling and bread quality. Typically they lack and are used in a formulation which lacks fungal alpha-amylase activity. Accordingly, these enzymes can be modified using the methods described herein.

Brewing

The mashing process used in traditional beer making consists of mixing crushed barley malt and hot water in a large circular vessel (a ‘mash copper’). Other cereals and cereal starches such as maize (corn), sorghum, rice and barley, or pure starch, are also optionally added to the mash. These are known as mash adjuncts. After mashing, the mash is filtered in a lauter tun. The resulting liquid, known as “sweet wort,” is then run off to the copper, where it is boiled with hops. The “hopped wort” is cooled and transferred to the fermentation vessels where yeast is added. After fermentation, the resulting “green beer” is matured before final filtration and bottling. Enzymes that are involved in these processes can be developed according to the methods of the invention and include the following.

Amyloglucosidase

Amyloglucosidase is used for producing “light” or low-carbohydrate beers.

Beta-Glucanase

Beta-glucanase is added to enhance glucan breakdown and/or to improve run-off and yield. Specialty versions (e.g., Finizym® from Novo Nordisk) are used to improve beer filtering properties and decrease haziness. Other specialty versions (e.g. Ultraflo® also from Novo Nordisk) are heat stable and flow stable and are used to improve filtration or worts, beers and intermediate liquors.

Alpha Amylases

Alpha amylases are used to increase the fermentability of worts.

Alpha-Acetolactate Decarboxylase

Alpha-acetolactate decarboxylase is used to decrease the time required for beer production time by reducing the level of the inhibitor diacetyl in the fermentation mix.

Neutral Proteases

Neutral proteases are used to catalyze release of sufficient nitrogen from malt and barley proteins to satisfy the nutritional needs of the fermenting yeast.

Pullanase

Pullanase is used for producing “light” or low-carbohydrate beers.

Alpha-Amylase

Alpha-amylase is used in the brewing process to enhance liquefaction of cereal adjuncts.

General Carbohydrase Complexes

General Carbohydrase complexes and mixtures are used for improving the filterability of wort and beer. In particular, carbohydrase and glucanase mixtures can be used to replace malt's own enzyme complement when brewing is done with barley.

Detergents

Proteases

Proteases are the most widely used enzymes in the detergent industry and are used to remove protein soils and stains derived from grass, blood, egg, human sweat, or the like. Most commercial proteases are suited to detergent formulations with pH values above 9. At low wash temperatures, subtilisin-derived proteases are particularly suitable. For bleach-containing formulations, oxidation-stable proteases (e.g., Everlase®) are commonly used. Accordingly, these enzymes can be modified using the methods described herein.

Lipases

Oil and fat-based stains historically have been more problematic than protein stains. The trend towards lower washing temperatures has further complicated the problem, especially for cotton and polyester blends.

A number of fungal lipases find use for alkaline cleaning applications conditions (up to pH 12 approximately) and are used over a broad temperature range. Some engineered variants exhibit improved performance at high ionic strength, low temperatures and/or high pH. Some also exhibit improved oil and fat removal properties. It would be desirable to develop lipases that exhibit improvement in combinations of properties. One aspect of the invention provides for lipases improved for all these properties plus high level secreted expression.

Amylases

Amylases are used to remove residues of starchy foods such as mashed potatoes, spaghetti, oatmeal porridge, custards, gravies and chocolate. Specialty versions have been developed for chlorine-containing and non-chlorine formulations and for use with and without bleach. Accordingly, amylases can be modified using the methods described herein.

Cellulases

The development of detergent enzymes has focused mainly on enzymes capable of removing stains by modifying the structure of cellulose fibrils such as those found on cotton and cotton blends. This has been observed to produce effects, such as color brightening, softening, and particulate soil removal.

Cellulases are most often of fungal origin. Enzymes of this category are generally supplied as a complex of active enzymes and used at the neutral to moderately alkaline pH for color brightening, softening, and removal of particulate soil. It works best on garments made of cotton and cotton blends. Monocompenent cellulases have also been developed to improve color brightening and fabric restoring properties of the complexed enzymes. Accordingly, these enzymes can be modified using the methods of the invention.

Bacterial Alkaline Proteases

Bacterial alkaline proteases are effective under neutral and mildly alkaline conditions (pH 7-10). These are useful for soaking preparations and liquid as well as powder detergents. Subtilisin-like proteases are typically effective under alkaline (pH 8-11) and medium-temperature wash conditions. Bleach-stabilized subtilisin and alkaline proteases have also demonstrated premier value in the marketplace. Variants and non-subtilisin alkaline proteases have been developed for use under extremely alkaline conditions (up to pH 12), such as Novo Nordisk's Esperase®. Accordingly, these enzymes can be modified using the methods described herein.

Alkaline Bacterial Amylase

Alkaline Bacterial amylases which work at (alkaline) pH values up to pH 11 and at high temperatures (up to 100° C.) are also desired and used in detergent applications. Accordingly, these enzymes can be modified using the methods described herein.

Neutral Bacterial Amylases

Neutral Bacterial Amylases are traditionally used at neutral to mildly alkaline conditions and at low and moderate wash temperatures. These enzymes are often used in granular form and in combination with subtilisins.

Food Functionality

Bacterial Proteases

Bacterial proteases are used for improving the functional, nutritional, and flavor properties of proteins. Accordingly, these enzymes can be modified using the methods described herein.

Fungal Exopeptidases and Endoproteases

Fungal complexes of exopeptidases and endoproteases are used for extensive hydrolysis of proteins. Fungal endo/exopeptidase boosts the fermentation of soy sauce. Accordingly, these enzymes can be modified using the methods described herein.

Trypsin

Trypsin is derived from porcine pancreas and can be improved using the methods of the invention.

Chrymotrypsin

Chrymotrypsin is present as a minor constituent in the porcine pancreas. Accordingly, the enzyme can be modified using the methods described herein.

Lipases

A 1,3-specific lipase is used, e.g., for improving the lipid palatability of pet food and for the production of cheese flavors. Accordingly, lipases can be modified using the methods described herein and screened for these properties.

Catalase

Catalase is used for the removal of residual hydrogen peroxide in foods and food ingredients. Accordingly, these enzymes can be modified using the methods described herein.

Bacterial Amylase

Bacterial amylase is used for reducing starch viscosity and can be improved using the methods described herein.

Multienzyme Complexes

Multienzyme complexes of carbohydrases, cellulases, hemicellulase, and xylanase are used, e.g., for breaking down plant cell walls. Accordingly, these enzymes can be modified using the methods described herein.

Lactase

Lactase preparations are used, e.g., for lactose-free or reduced lactose milk and yogurt. For example, beta-galactosidases are described in, e.g., U.S. Pat. No. 5,736,374. Accordingly, these enzymes can be modified using the methods described herein.

Phospholipase

Phospholipase is used for partial hydrolysis of phospholipids and can be developed according to the methods described herein.

Leather

The processing of skin and hides into leather has been based on enzymes since 1908 when Otto Röhm patented the first standardized bate containing pancreatic enzymes. Before the hides and skins can be tanned, protein and fat between the collagen fibres must be partially or totally removed. The protein can be removed by proteases and the fat can be removed by lipases, as well as by surfactants and organic solvents. Specific enzymes used for leather treatment which can be developed according to the methods described herein include the following:

Proteases

Proteases are used mainly in the soaking, bating, and enzyme-assisted unhairing steps. Salt stable proteases are commonly used to rehydrate dried and salted hides. Trypsin and trypsin-like protease, and neutral and alkaline proteases, are used for neutral and alkaline bating of hides and skins.

Lipases

Lipases are used for degreasing by hydrolyzing fat on the flesh side and inside the skin structure. Lipases reduce the need for surfactants or organic solvents and this has clear environmental benefits. For example, alkaline and acid lipases are used for degreasing hides and skins.

Oils & Fats

The food industry uses enzymes to modify food-grade oils and fats. Some uses are proven sufficiently that enzyme products are now on the market to address these applications. The following provides a brief discussion of such approaches:

Fat Modification

Fat modification typically involves the specific esterification or de-esterification of triglyceride 1,2 and 3 positions. This allows processors to produce “custom-made” fats and oils. These include oils, such as palm oil which provides an alternative to expensive supply limited cocoa butter for chocolate production. Palm oil is upgraded in a reaction with stearic acid using enzymatic interesterification. Palm oil can also be upgraded by a large number of other enzymatic modifications and used in a wider variety of applications. Furthermore, the melting point, spreadability, shelf-life or nutritional properties of a natural fat or oil can be modified, such as in margarine production. Accordingly, these enzymes can be modified using the methods described herein.

Ester Synthesis

Ester synthesis, including the production of fatty esters has traditionally been done by chemical catalysis. Poor yields and unwanted side-reactions, however, limit value and utility. Enzymes offer an advantage due to low temperature of catalysis and high selectivity. Additionally, flavors and fragrances often consist of esters, as do surfactants in cosmetic products (e.g. moisturizing creams and shampoos). Esterases are described in, e.g., PCT publication No. WO 98/14594. Accordingly, these enzymes can be modified using the methods described herein.

Lysolecithin

Lecithin is a by-product of seed oil refining that can be used as an emulsifier. Esterases are used to produce lysolecithin. The latter has superior emulsifying properties to normal lecithin and finds importance in margarines and cosmetics.

Specific enzymes of interest in this area include, e.g., phospholipase for the modification of lecithins; immobilized lipase for ester synthesis; immobilized 1,3-specific lipase for the production of tailor-made oils, fats and esters; 1,3-specific lipase for the hydrolysis of esters; 1,3-specific lipase for the hydrolysis of esters; and non-specific lipase for the hydrolysis of esters. Accordingly, these enzymes can be modified using the methods described herein.

Pulp & Paper

In general, bacterial and fungal amylases have been used for low-temperature modification of starch. Cellulase preparations are used for the de-inking of mixed office waste materials, such as for recycling. Enzymes, such as xylanase preparations are used, e.g., for reducing the need of bleaching chemicals when bleaching kraft pulp. Other enzymes such as resinase are used to eliminate pitch/resin-related problems. Accordingly, these enzymes can be modified using the methods described herein.

Starch Production

Enzymes of interest in this area include the following: amyloglucosidase—for conversion dextrin into glucose; bacterial amylase—for traditional two-step liquefaction of starch to dextrin; dextranase—for breaking down dextran in raw sugar juice; fructoamylase—for hydrolysis of inulin to fructose; fungal alpha amylase—for making high maltose and special glucose syrups; bacterial (malto)alpha amylase—for making high maltose and special glucose syrups; pullulanase—for debranching starch after liquefaction and reducing the oligosaccharide content of glucose syrups; xylanase—for improved wheat gluten/starch separation; glucose isomerase—for converting glucose into fructose; heat-stable bacterial alpha-amylase—for one-step liquefaction of starch to dextrin; alpha amylase-heat-stable bacterial alpha-amylase for one-step liquefaction of starch to dextrin; and heat stable cyclomaltodextrin glucanotransferase (CGTase)—for cyclodextrin production. Any of these enzymes can be modified and selected for improved properties according to the methods described herein.

Textiles

In recent years, the use of enzymes has resulted in improved production and finishing methods for a number of fabrics. For example, the use of amylase to remove starch sizing agents is among the oldest enxyme-based applications within textile manufacturing. Moreover, coating the longitudinal threads of fabrics (i.e. the “warp”) with starch is often used to prevent damage or breaking of these threads during the weaving process.

As a class, few enzymes have found as high a value in fabric finishing as the cellulases. In polishing operations, such enzymes are used to remove pills and restore a smooth, high luster look to cotton-based fabrics. More recently, cellulases have proven effective at enhancing and even creating the “stone-washed” look which traditionally required the abrasive action of pumice stones.

Hydrogen peroxide has to be removed before dyeing. Catalases are used for degrading residual hydrogen peroxide after the bleaching of cotton.

Proteases are used for wool treatment and the degumming of raw silk.

Any of these enzymes can be modified according to the methods described herein.

Desizing of Cotton Fabric

For almost a century, starch has been a favored sizing agent in many areas of the fabric production industry. However, the sizing agents must be removed prior to bleaching, dyeing or other finishing steps. Enzymes capable of mediating the breakdown of starch are often capable of removing the carbohydrate without affecting other micro- or macro-properties of the yarn or woven fabric. Most commonly, desizing operations are conducted using a jigger which allows fabric from one roll to be passed through a bath and rewound on another roll. The bath generally contains hot water hot water (80-95° C.) which allows the starch to gelatinize. For desizing, the liquor is then adjusted to pH 5.5-7.5 and temperatures of 60-80° C. depending on the enzyme. Degraded starch (in the form of dextrins) is then removed by washing at 90-95° C. for two minutes.

Enzymes produced according to the methods described herein which allow this to be a smoother more continuous process such as by eliminating the need for adjusting the temperature or pH between steps can be produced.

In some cases, enzymes facilitate conversion from a batch type process to a continuous one. In some such operations, however, desizing on pad rolls is continuous in terms of the passage of the fabric but then requires a holding time of 2-16 hours at 20-60° C. due to low temperature and slow speed of many low-temperature alpha-amylases. The higher the temperature stability of amylases, the more likely it becomes that the desizing reactions can be conducted, such as in steam chambers at 95-100° C. Accordingly, thermostable enzymes produced by the methods herein are a feature of the Invention.

Denim Finishing

Finish of denim has become an industry of its own within the textile and garment industry. Most denim jeans or other denim garments are subjected to a wash treatment to give them a slightly worn look. In the traditional stone-washing process, the abrasive action of lightweight pumice stones on the blue denim surface in facilitated in specially modified washng machines. The process requires the later removal of rocks, dust and debris and often results in unwanted damage to the product. Today, denim finishers often opt instead for the use of cellulases to accelerate the abrasion by loosening the indigo dye on the denim. Even a small dose of enzyme can typically replace several kilograms of stones, allowing the use of fewer stones and lessening damage to garments. With stone-free processes, the removal of dust and small stones from the finished material or garment becomes almost a non-issue, minimizing the generation of both sediment and waste water.

The mechanism of stone washing relies on the priniciple that denim garments are dyed with indigo. The dye adheres primarily to the surface of the yarn. The cellulase molecule binds to an exposed fibril on the surface of the yarn and hydrolyzes it. Importantly, such action leaves the interior part of the cotton fiber (responsible for the strength of the yarn) intact. When cellulases partially hydrolyze the surface of the fiber surface, however, it results in the release of some of the indigo from the surface, thereby creating the characteristic “bleached” or stone-washed appearance.

Both neutral cellulases acting at pH 6-8 and acid cellulases acting at pH 4-6 are used for the abrasion of denim. There are a number of cellulases available, each with its own special properties. These can be used either alone or in combination in order to obtain a specific look. Research in the denim finishing is focused on preventing or reducing redeposition of dye on the enzyme-treated surface. At low pH values (pH 4-6) redeposition rates are high. At near neutral pHs, it is much less significant. Therefore, interest in discovering or otherwise generating neutral cellulases is high and a number have been commercialized. These enzymes have resulted in an increase in the variety of denim finishes available. For example, low damage denim “bleaching” is now possible and is being used to create lighter denim garments. Improving both activities, stabilities, fibril specificity, and pH and thermal properties of current enzymes can be performed according to the methods described herein for these high fashion applications.

Cellulases for Polishing of Cotton Fabric

Microfibrils (observed as hairs or fuzz) protruding from the surface of yarn or a fabric provide an ideal substrate for certain classes of cellulases due both to the extended structure of the fibril and its exposure to solvent. Attack of these microfibrils by cellulase weakens them allowing them to break off from the main body of the fiber and thus leave a smoother surface. An observable ball of fuzz on a garment or fabric surface is generally referred to as a “pill” in the textile trade. Pilling of yarns, fabrics or garments upon use result in an unattractive, knotty fabric appearance and thereby constitute a quality control issue at each stage of the process leading up to and including manufacture of a finished garment. Depending on the yarn and the enzyme used, polishing the fabric with cellulases can both remove existing pills and reduce pilling tendency in downstream operations. Furthermore, removal of fuzz results in a softer and smoother feel, and superior color brightness.

Enzymes for Wool and Silk Finishing

Polishing of yarn, fabric and garment surfaces works similarly for materials comprised of non-cellulosic fibers as well. For example, wool and silk are proteinaceous (amino acid-based fibers) and are polished via treatment with a suitable proteases. Such enzymatic treatment reduces pilling and increases softness of garments made from the treated fabrics. Proteases are also used to treat silk both for degumming of raw silk and depilling silk-containing garments and fabrics. Accordingly, these enzymes can be modified using the methods described herein.

Scouring

Before cotton yarn or fabric can be dyed, the non-cellulosic components found in native cotton must be removed. This complete removal of unwanted components, referred to as scouring, gives a fabric high, even wettability so it can be bleached and dyed successfully. Today, highly alkaline chemicals such as sodium hydroxide are used for scouring. These chemicals not only remove the impurities but also attack the cellulose leading to a reduction in strength and loss of weight of the fabric. Furthermore, the resulting waste water has a high COD (chemical oxygen demand), BOD (biological oxygen demand) and salt content. Accordingly, these enzymes can be modified using the methods described herein.

Recently, an alkaline pectinase (e.g., Novo Nordisk's BioPrep™ 3000 L) was introduced. This enzyme promises to reduce environmental impact, decrease weight loss and strength loss due to the scouring process and leave the cellulosic structure intact and, in most cases, work out more economical to use. Accordingly, these enzymes can be improved using the methods described herein.

Wine & Fruit Juice

Pectin is an important natural biopolymer which helps hold plant cell walls together. When producing juice from any type of fruit or berry a manufacture must contend with the “gummy” properties of this very important natural polymer. As a fruit ripens, the hard, insoluble protopectin begins to undergo partial hydrolysis, resulting in decreased molecular weight and increased, but partial solubility. This solubility allows some of the pectin to pass into the juice during the pressing of fruits and berries. By doing so, it increase viscosity and decreases juice recovery (yield) in downstream operations. While the pectin is difficult to remove by filtration and other cost effective processing methods, its presence in the juice results in both cloudiness (lack of clarity) and taste alteration.

Pectinases

Addition of pectinases to the fruit pulp prior to pressing facilitates the release of the juice, increases yield and pressing capacity. Moreover, complete depectinization by treatment with additional pectinase(s) preparations ensure good clarification and filtration of the juices through downstream operations and good stability for the juices produced. Accordingly, these enzymes can be modified using the methods described herein.

Other Enzymes

Some juices, such as apple juice contain high amounts of starch, especially early in the growing season. To produce clear, stable juice or concentrate, this starch must be degraded. This is achieved by addition of amylases and pectinases together during depectinization of the juice. Cellulases are also important for improving juice yields and color extraction in certain berry extract. Other polysaccharides such as araban can also be selectively degraded by specific degradative enzymes. Accordingly, these enzymes can be modified using the methods described herein.

Enzymes for the Citrus Industry

Special pectolytic enzyme preparations (Citrozym®, Citropex™) are used in the citrus industry. In the pulp wash process, enzymes are used to reduce viscosity in order to avoid jellification of pectin during concentration. Tailor-made pectolytic enzymes are used for the clarification of citrus juices (particularly lemon and lime juice), for the recovery of essential oils and the production of highly turbid extracts from the peels of citrus fruit. These cloudy concentrates are used in the manufacture of soft drinks.

The enzymatic peeling of citrus fruit is a relatively new application for the production of fresh peeled fruit, fruit salads and segments. Enzymatic treatment with Peelzym™ results in citrus segments with improved freshness as well as texture and appearance compared with the traditional process using caustic soda. Accordingly, these enzymes can be modified using the methods described herein.

Special Enzymes for Winemakers

The ideal enzyme preparations for winemaking are different to those for fruit juice processing. In winemaking, very specific enzyme activities are required in order to obtain the desired effect while at the same time ensuring the best quality.

In fruit juice processing, the enzymes are inactivated very shortly after they have done their job, for example by pasteurization. In winemaking, no such heat treatment takes place. The enzymes, therefore maintain their activity over a longer period. Side activities that may be beneficial for fruit juice processing can be less desirable for winemaking as they may negatively influence wine quality during storage. Specific enzyme preparations for winemaking have been developed in order to improve wine quality while at the same time bringing about the desired technological advantages.

In winemaking, one aim is to extract as many flavour compounds as possible. In the case of red wine, color extraction is also very important.

One problem very specific to winemaking is the extremely difficult clarification and filtration of wines made from grapes attacked by the fungus Botrytis cinerea. The Botrytis fungus produces beta-glucans (polymers of glucose with a high molecular weight) which pass into the wine. These large molecules hinder clarification and rapidly clog filters. The troublesome beta-glucans can easily be removed by adding a highly specific beta-glucanase to the wine.

Research into the chemical composition of grapes is opening up new enzyme applications. One example is the Novo Nordisk enzyme Novoferm® 12 for aroma liberation. The glycosidases in Novoferm® 12 hydrolyze terpenyl glycosides (also known as bound terpenes) found in grapes. Terpenes are released and these are one of the important constituents of the bouquet. Winetasters can usually detect a noticeable improvement in the bouquet after treatment with Novoferm® 12.

Wine

Pectinase

Unique pectinases preparations are used for grape maceration in red wine making and thermovinification. They are also used for grape maceration and clarification in white and rose wine making. Accordingly, these enzymes can be modified using the methods described herein.

Beta-Glucanase or Pectinase/Glucanase Blends

These enzymes are used, e.g., for aroma enchancement in young wines, for improvement of aging and filtration in young wines, and for improvement of filtration of young wines with Botrytis glucan. Accordingly, these enzymes can be modified using the methods described herein.

Fruit Juice

Mash Treatment

There are a variety of different pectinases containing a range of hemicellulotic side activities. They are used, e.g., for apple and pear mash treatment resulting in higher yield and capacity. Accordingly, these enzymes can be modified using the methods described herein.

Pomace Treatment

Pectinase preparations with a relatively broad spectrum of side activities, such as cellulases and hemicellulases, are used for enzymatic pomace treatment to increase yield. Accordingly, these enzymes can be improved using the methods described herein.

Juice Depectinization

A combination of pectintranseliminase, polygalacturonase and pectinesterase with arabanase side activity in various strengths for juice treatment. Accordingly, these enzymes can be modified using the methods described herein.

Starch Degradation of Juice

Amyloglucosidase is often used for hot treatment of juice to break down the starch. Accordingly, theremostable amyloglucosidaes produced according to the methods described herein are a feature of the invention.

Juice Filtration

A pectinase preparation with rhamnogalacturonase side activity can be used to increase the filterability (ultra and microfiltration) of juice. Accordingly, these enzymes can be modified using the methods described herein.

Berry Treatment

Pectinase preparations typically include pH spectrums particularly well suited to berries which maximixes yield and improves color extraction. Accordingly, these enzymes can be modified using the methods described herein.

Membrane Cleaning

A multi-active enzyme preparation can be used as a cleaning agent to remove colloids from membranes. Accordingly, these enzymes can be modified using the methods described herein.

Cellobiases

A cellobiase preparation can be used to prevent the formation of cellobiose in fruit juice concentrates. Accordingly, these enzymes can be modified using the methods described herein.

Citrus

A hemicellulase-pectinase is used, e.g., for improved recovery of citrus essential oils, reduction in clear juices, and other juice clarification. Pectinase preparations are used, e.g., for extraction and viscosity reduction in cloudy citrus juices. A pectinase-arabanase is commonly used for lemon juice clarification.

In conclusion, any of the many targets noted above can be modified according to the methods of the present invention, optionally including selection for one or more activity as noted. In all cases, new or improved properties, e.g., corresponding to those noted above can be selected for.

Upstream/Downstream Processing

The template nucleic acids, isolated nucleic acid fragments and chimeric nucleic acid sequences produced by the methods described herein can optionally be used as substrates for various upstream and/or downstream processing steps. For example, the chimeric sequences or isolated fragments can be amplified by PCR or a comparable technique, as discussed above. Additionally, encoded expression products of amplified chimeric nucleic acid sequences can be selected for desired traits or properties following, e.g., in vitro expression. The chimeric nucleic acid sequences can also optionally be introduced into suitable host cells and be expressed to provide, e.g., an enzyme or structural protein to the cells.

Other processing options can include fragmenting the amplified chimeric nucleic acid sequences by, e.g., nuclease digestion to provide chimeric nucleic acid sequence fragments. Thereafter, chimeric sequence fragments or isolated nucleic acid fragments can be used, e.g., as substrates for further recombination (e.g., additional single-stranded nucleic acid template-mediated recombination, reiterative nucleic acid recombination, and the like), as substrates for the methods of isolating a set of nucleic acids fragments, and the like. Similarly, the chimeric nucleic acids can be used as templates according to the methods herein.

The chimeric nucleic acid sequences or isolated nucleic acid fragments can also be used as substrates for various mutagenic methods, such as recombination, cassette mutagenesis, site-directed mutagenesis, chemical mutagenesis, error-prone PCR, and the like. These and other techniques for creating diversity are well-known and set forth in the references below.

Recombination and Mutagenesis

A variety of diversity generating protocols are available and described in the art. The procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics. These methods can be used in combination with any of the methods herein, either to provide substrates for the methods herein, or to further modify, mutate or evolve any chimeric nucleic acid produced herein, or both.

While distinctions and classifications are made in the course of the ensuing discussion for clarity, it will be appreciated that the techniques are often not mutually exclusive. Indeed, the various methods can be used singly or in combination, in parallel or in series, with each other or with the methods herein, to generate diverse sequence variants and to screen for desirable activity in such diverse variants.

The result of any of the diversity generating procedures described herein can be the generation of one or more nucleic acids, which can be selected or screened for nucleic acids that encode proteins with or which confer desirable properties. Following diversification by one or more of the methods herein, or otherwise available to one of skill, any nucleic acids that are produced can be selected for a desired activity or property. This can include identifying any activity that can be detected, for example, in an automated or automatable format, by any of the assays in the art as discussed below. A variety of related (or even unrelated) properties can be evaluated, in serial or in parallel, at the discretion of the practitioner.

Descriptions of a variety of diversity generating procedures for modifying nucleic acid sequences are found the following publications and the references cited therein: Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp. 447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270:1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity, which can be practiced in combination with other diversity generation methods including those noted herein, include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundström et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications, and EPO publications; U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/13487 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Certain U.S. applications provide additional details regarding various diversity generating methods, including “SHUFFLING OF CODON ALTERED GENES” by Patten et al. filed Sep. 28, 1999, (U.S. Ser. No. 09/407,800); “EVOLUTION OF WHOLE CELLS AND ORGANISMS BY RECURSIVE SEQUENCE RECOMBINATION”, by del Cardayre et al. filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-BASED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); and “METHODS OF POPULATING DATA STRUCTURES FOR USE IN EVOLUTIONARY SIMULATIONS” by Selifonov and Stemmer, filed Jan. 18, 2000 (PCT/US00/01138).

In brief, several different general classes of sequence modification methods, such as mutation, recombination, etc. are applicable to the present invention and set forth, e.g., in the references above. The following exemplify some of the different types of preferred formats for diversity generation that are optionally adapted to the present invention to create further diversity in, e.g., the chimeric nucleic acid or gene sequences, or in the substrates for recombination (e.g., single-stranded nucleic acid templates, fragments, etc.) discussed herein, to produce new proteins or other expression products with improved properties.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., PCT/US99/15972 by del Cardayre et al., also entitled “Evolution of Whole Cells and Organisms by Recursive Sequence Recombination.”

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-BASED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202); “METHODS OF POPULATING DATA STRUCTURES FOR USE IN EVOLUTIONARY SIMULATIONS” by Selifonov and Stemmer (PCT/US00/01138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579).

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids which correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) “METHODS OF POPULATING DATA STRUCTURES FOR USE IN EVOLUTIONARY SIMULATIONS” by Selifonov and Stemmer (PCT/US00/01138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579). Extensive details regarding in silico recombination methods are found in these applications. This methodology is generally applicable to the present invention in providing, e.g., for template-mediated recombination in silico and/or the generation of corresponding nucleic acids or proteins.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods which result in the alteration of individual nucleotides or groups of contiguous or non-contiguous nucleotides can be favorably employed to introduce nucleotide diversity. Many mutagenesis methods are found in the above-cited references; additional details regarding mutagenesis methods can be found in the following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “METHODS FOR GENERATING AND SCREENING NOVEL METABOLIC PATHWAYS,” and Thompson, et al., (1998) U.S. Pat. No. 5,824,485 METHODS FOR GENERATING AND SCREENING NOVEL METABOLIC PATHWAYS) and their use to identify protein activities of interest has been proposed (In addition to the references noted above, see, Short (1999) U.S. Pat. No. 5,958,672 “PROTEIN ACTIVITY SCREENING OF CLONES HAVING DNA FROM UNCULTIVATED MICROORGANISMS”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above descibed procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215:471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “PRODUCTION OF ENZYMES HAVING DESIRED ACTIVITIES BY MUTAGENESIS.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described herein.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods can be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 51F to lambda repressor” J. Biol. Chem. 264:13355-60); and “Walk-Through Mutagenesis” (Crea, R; U.S. Pat. Nos. 5,830,650 and 5,798,208, and EP Patent 0527809 B 1.

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides. Any of the methods in the references above can be used in combination with any method herein, to provide substrates to the reactions noted herein, or to further modify the chimeric nucleic acids produced according to the methods herein.

Introduction of Nucleic Acid Sequences into the Cells of Organisms of Interest

In certain embodiments of the present invention, chimeric nucleic acids or other sequences are introduced into the cells of particular organisms of interest. There are several well-known methods of introducing target nucleic acids into, e.g., bacterial cells, any of which may be used in the present invention. These include: fusion of the recipient cells with bacterial protoplasts containing the DNA, electroporation, projectile bombardment, and infection with viral vectors, etc. Bacterial cells can be used to amplify the number of plasmids containing DNA constructs of this invention.

Bacteria are typically grown to log phase and the plasmids within the bacteria can be isolated by a variety of methods known in the art (see, for instance, Sambrook). In addition, a plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAexpress Expression System™ from Qiagen). The isolated and purified plasmids are then further manipulated to produce other plasmids.

Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith, Gene 8:81 (1979); Roberts, et al., Nature, 328:731 (1987); Schneider, B., et al., Protein Expr. Purif 6435:10 (1995); Ausubel, Sambrook, Berger (all supra). A catalogue of Bacteria and Bacteriophages useful for cloning is provided, e.g., by the ATCC, e.g., The ATCC Catalogue of Bacteria and Bacteriophage (1992) Gherna et al. (eds) published by the ATCC.

Additional basic procedures for sequencing, cloning and other aspects of molecular biology and underlying theoretical considerations are also found in Watson et al. (1992) Recombinant DNA Second Edition Scientific American Books, NY. Furthermore, a wide variety of cloning kits and associated products are commercially available from, e.g., Pharmacia Biotech, Stratagene, Sigma-Aldrich Co., Novagen, Inc., Fermentas, and 5 Prime→3 Prime, Inc.

Selection of a Desired Trait or Property

The present invention includes various recombination and nucleic acid isolation methods mediated by single-stranded nucleic acid templates to derive, e.g., chimeric nucleic acid sequences, isolated nucleic acid fragments, and the like. These products can subsequently be further recombined or otherwise bred for desired traits or properties. There are various “breedable” properties for which, e.g., evolved biocatalysts can be selected including assorted kinetic constants, stability, selectivity, inhibition profiles, altered substrate specificity, increased enantioselectivity, increased activity, increased gene expression, activity under diverse environmental conditions (i.e., increased thermostability, increased activity in various organic solvents, pH tolerance, etc.), and the like. Generally, one or more recombination cycle(s) is/are optionally followed by at least one cycle of selection for molecules having one or more of these or other desired traits or properties. A wide variety of desirable properties to be screened for are noted above and others will be apparent to one of skill.

If a recombination cycle is performed in vitro, the products of recombination, i.e., recombinant or evolved nucleic acids, are sometimes introduced into cells before the selection step. Recombinant nucleic acids can also be linked to an appropriate vector or to other regulatory sequences before selection. Alternatively, products of recombination generated in vitro are sometimes packaged in viruses (e.g., bacteriophage) before selection. If recombination is performed in vivo, recombination products may sometimes be selected in the cells in which recombination occurred. In other applications, recombinant segments are extracted from the cells, and optionally packaged as viruses or other vectors, before selection.

The nature of selection depends on what trait or property is to be acquired or for which improvement is sought. It is not usually necessary to understand the molecular basis by which particular recombination products have acquired new or improved traits or properties relative to the starting substrates. For instance, a gene has many component sequences, each having a different intended role (e.g., coding sequences, regulatory sequences, targeting sequences, stability-conferring sequences, subunit sequences and sequences affecting integration). Each of these component sequences are optionally varied and recombined simultaneously. Selection is then performed, for example, for recombinant products that have an increased ability to confer activity upon a cell without the need to attribute such improvement to any of the individual component sequences of the vector.

Depending on the particular protocol used to select for a desired trait or property, initial round(s) of screening can sometimes be performed using bacterial cells due to high transfection efficiencies and ease of culture. However, yeast, fungal or other eukaryotic systems may also be used for library expression and screening when bacterial expression is not practical or desired. Similarly, other types of selection that are not amenable to screening in bacterial or simple eukaryotic library cells, are performed in cells selected for use in an environment close to that of their intended use. Final rounds of screening are optionally performed in the precise cell type of intended use.

When further improvement in a trait is sought, at least one and usually a collection of recombinant products surviving a first round of screening/selection are optionally subject to a further round of recombination. These recombinant products can be recombined with each other or with exogenous segments representing the original substrates or further variants thereof. Again, recombination can proceed in vitro or in vivo. If the previous screening step identifies desired recombinant products as components of cells, the components can be subjected to further recombination in vivo, or can be subjected to further recombination in vitro, or can be isolated before performing a round of in vitro recombination. Conversely, if the previous selection step identifies desired recombinant products in naked form or as components of viruses, these segments can be introduced into cells to perform a round of in vivo recombination. The second round of recombination, irrespective of how performed, generates additionally recombined products which encompass more diversity than is present in recombinant products resulting from previous rounds.

The second round of recombination may be followed by still further rounds of screening/selection according to the principles discussed for the first round. The stringency of selection can be increased between rounds. Also, the nature of the screen and the trait or property being selected may be varied between rounds if improvement in more than one trait or property is sought. Additional rounds of recombination and screening can then be performed until the recombinant products have sufficiently evolved to acquire the desired new or improved trait or property.

Multiple cycles of recombination can be performed to increase library diversity before a round of selection is performed. Alternately, where the library is diverse, multiple rounds of selection can be performed prior to recombination methods.

In the context of a particular experiment, a variety of related (or even unrelated) properties can be selected for using any available assay. For example, screening assays for an evolved dehalogenase activity can be performed, e.g., by detecting protons, hydronium ions or halide ions liberated upon hydrolysis of, e.g., carbon-halogen bonds in reactant or substrate molecules. Other suitable techniques can include alcohol dehydrogenase-linked enzyme assays, fluorescence resonance energy transfer (FRET) assays, gas chromatography mass spectroscopy (GCMS) analysis, or the like.

Screening is optionally performed using a plate assay. For example, cells expressing a library of, e.g., the at least substantially full-length chimeric nucleic acid sequences of the invention are optionally plated onto a suitable medium (e.g., nutrient agar) containing a substrate which develops zones of clearing or color change (“halos”) surrounding cells expressing, e.g., an active enzyme. For example, one well-known plate assay substrate for protease is casein (e.g., 1-2% skim milk powder in agar; see, e.g., Ness J. E. et al. (1999) Nature Biotechnol. 17:893-896). A variety of colorimetric substrates suitable for plate assays are commercially available; for example, azo-labeled or azurine-crosslinked (AZCL)-polysaccharides and polypeptides and can be used as substrates in plate assays according to protocols supplied by the manufacturer (Megazyme; Wicklow, Rep. of Ireland). Exemplary enzymes and substrates include: AZCL-Amylose (for the assay of alpha-amylases); AZCL-Arabinoxylan, AZCL-Xylan (xylanases); AZCL-Barley Beta-Glucan, AZCL-HE-Cellulose, AZCL-Xyloglucan (cellulases); AZCL-Pullulan (pullulanases); AZCL-Dextran, AZCL-Curdlan (endo-glucanases); AZCL-Collagen and AZCL-Casein (proteases).

Screening may also be performed using a filter assay. Cells expressing a library of, e.g., the at least substantially full-length chimeric nucleic acid sequences are optionally plated onto a pair of filters placed atop a suitable medium (e.g., nutrient agar) and incubated under suitable conditions for the enzyme to be secreted. The pair of filters include a lower protein-binding filter and, on top of that, an upper filter exhibiting a low protein binding capability. Cells are retained on the upper filter, while secreted enzymes pass through the upper filter and bind to the lower filter. The lower filter may be any protein binding filter, e.g., nylon or nitrocellulose. The upper filter carrying the colonies of the expression organism may be any filter that has no or low affinity for binding proteins, e.g. cellulose acetate or Durapore™.

Following incubation to express secreted enzymes (e.g., one to several days), the lower filter is separated from the upper filter. The lower filter is subjected to assays for the desired enzymatic activity, and the corresponding cellcolonies present on the upper filter are identified. The lower filter may be pretreated with any of the conditions to be used for screening, or may be treated during the assay itself.

Enzymatic activity on the filter may be detected by a dye, fluorescence, precipitation, pH indicator, or any other known technique for detection of enzymatic activity. A wide variety of assays suitable for detection of specific enzymes on filters and gel-based formats (e.g., agarose, agar, gelatin, polyacrylamide, etc.) is provided, e.g., in Manchenko, G. P., Handbook of Detection of Enzymes on Electrophoretic Gels (CRC Press, Boca Raton, Fla., 1994) and references cited therein.

The conditions for screening may be chosen to correspond with the desired properties or uses of the enzymes being screened. Desired properties for enzymes used in commercial or industrial applications include, but are not limited to, thermal stability, pH (e.g., acid or alkaline) stability, oxidative stability, solvent stability, builder(chelator) stability, and/or detergent(surfactant) stability. These properties can be assayed by methods known in the art. For example, using the filter assay format described above, the filter containing bound enzyme variants can be incubated in solutions containing, e.g., low or high pH buffer, calcium, detergents, EDTA, peroxide, etc., at a desired temperature for a desired length of time, prior to assaying the filter-bound enzymes for activity.

For example, in screening for enzymes for use in the cleaning industry, it may be relevant to screen for an enzyme (for example, a lipase) having increased stability in alkaline conditions, an increased temperature stability, and increased stability towards chelators and surfactants. To illustrate, a filter with bound lipase variants is incubated in a buffer at pH 10 containing 2 mM EDTA and detergent at 60° C. for a specified time, rinsed briefly in deionized water and placed on an olive-oil agarose matrix for activity detection. The agarose matrix contains an olive oil emulsion (2% PVA:olive oil=3:1) and Brilliant Green indicator (0.004%). Active lipase is indicated by the presence of blue-green spots. The incubation conditions are chosen to be such that activity due to a predetermined control lipase (e.g. a parental lipase) can barely be detected. Improved lipase variants show, under the same conditions, increased color intensity on the detection plate.

Likewise, in screening for enzymes for use in the paper and pulp industry, it may be relevant to screen for acid-stable enzymes having an increased temperature stability. This may be performed by incubating the filters in a buffer at acidic pH (e.g., of about pH 4) and at higher temperature before or during the assay.

For screening for variants with an activity optimum at a lower temperature and/or over a broader temperature range (which is desirable, e.g., for low-temperature fabric washing applications), the filter with bound variants is placed directly on the activity detection plate and incubated at the desired temperature (e.g., about 10° C. or about 15° C.) for a specified time. After this time activity due to the control enzyme can barely be detected, while variants with optimum activity at a lower temperature will show increased activity.

Alkaline stability can be measured, for example, as the residual enzyme activity following incubation of a test enzyme for a predetermined time (e.g., about 10 minutes) at a predermined alkaline pH (e.g., a pH about 10) as compared to the residual activity of a control enzyme reaction incubated at, e.g., neutral pH (or, the optimal pH for that particular enzyme) but under otherwise equivalent conditions. Likewise, acid stability can be measured as above but at a predetermined acidic pH (e.g., a pH of about 4).

Thermal stability can be measured, for example, as the residual enzyme activity following incubation of a test enzyme for a predetermined time (e.g., about 5 minutes) at a predermined temperature (e.g., about 70° C.) as compared to the residual activity of a control enzyme reaction incubated at, e.g., about 25° C., and otherwise equivalent conditions.

Oxidative stability can be measured, for example, as the residual enzyme activity following incubation of a test enzyme for a predetermined time (e.g., about 5 minutes) in the presence of a predermined amount of oxidizing agent (e.g., hydrogen peroxide, or diperdodecanoic acid (DPDA)) as compared to the residual activity of a control enzyme reaction incubated without oxidizing agent but under otherwise equivalent conditions.

Solvent stability can be measured, for example, as the activity of a test enzyme assayed in the presence of a predetermined amount of solvent (e.g., 35% dimethylformamide (DMF)) as compared to the activity of the enzyme assayed in the absence of the solvent but under otherwise equivalent conditions. Likewise, detergent stability can be measured, for example, as the activity of a test enzyme assayed in the presence of a predetermined amount of detergent as compared to the activity of the enzyme assayed in the absence of the detergent but under otherwise equivalent conditions.

Libraries generated via the methods described herein may be screened for specified enzyme activities, e.g., for one or more of the six IUB classes; oxidoreductases, transferases, hydrolases, lyases, isomerases and ligases. The recombinant enzymes which are determined by sequence or activity to be positive for one or more of the IUB classes may then be rescreened for a more specific enzyme activity. Alternatively, bacterial colonies containing a functional open reading frame may be identified by including an in-frame downstream cistron encoding an easily detectable protein such as green fluorescent protein. Colonies expressing complete open reading frames may be selected for more detailed kinetic and physical characterization.

Alternatively, the library may be screened directly for a more specialized enzyme activity. For example, instead of generically screening for hydrolase activity, the library may be screened for a more specialized activity, i.e. the type of bond on which the hydrolase acts; e.g. a surrogate substrate or even the specific substrate of interest. Thus, for example, the library may be screened to ascertain those hydrolases which act on one or more specified chemical functionalities, such as: (a) amide (peptide bonds), i.e. proteases; (b) ester bonds, i.e. esterases and lipases; (c) acetals, i.e., glycosidases etc.

The clones which are identified as having the specified enzyme activity may then be sequenced to identify the DNA sequence encoding an enzyme having the specified activity. Thus, in accordance with the present invention it is possible to isolate and identify: (i) DNA encoding an enzyme having a specified enzyme activity, (ii) enzymes having such activity (including the amino acid sequence thereof) and (iii) combinatorial properties which may each be essential for commercial viability. The invention also provides methods for producing recombinant enzymes having such desired activities.

The present invention may be employed, for example, to identify new enzymes having the following activities and/or uses. For examples, enzymes having lipase and/or esterase activity, such as enantio- and/or chemoselective hydrolysis of polyesters, esters (lipids), thioesters, proteins, polyamides, amides, or the like may be used, e.g., to resolve racemic mixtures; in the synthesis of optically active acids or alcohols from meso-diesters; in the synthesis, polymerization and/or resolution of acid-SCoA esters; and for the polymerization and/or depolymerization of activated and nonactivated hydroxy esters. Enzymes with lipase and/or esterase activity may used, e.g., for selective syntheses, such as regiospecific and enantiospecific hydrolysis of carbohydrate esters; selective hydrolysis of cyclic secondary alcohols; selective hydrolysis polyhydroxy esters. They can also be screened for an ability to synthesize optically active esters, lactones, acids, alcohols, e.g., the transesterification of activated/nonactivated esters; interesterification; the synthesis of optically active lactones from hydroxyesters; the synthesis of optically active hydroxyester polymers and oligomers; or the regio- and enantioselective ring opening of anhydrides. Lipases and/or esterase enzymes can also be used in detergents. They can be screened for optimization of temperature range and stability; optimization of fabric and soil binding properties; optimization of stability and/or activity in presence of one or more surfactants, builders, stabilizers and chelators used in domestic or industrial detergent formulations; and for the enhancement of expression and/or yield of commercial enzyme preparations or the cell expressing such an enzyme, including but not limited to altering the preferred production host to allow for use of less expensive raw materials. Enzymes with lipase and/or esterase activity may also be used, e.g., in fat/oil conversions and in cheese ripening.

Enzymes exhibiting a protease activity may be selected for, e.g., an ability to synthesize esters, amides, and polyamides, e.g., for use in the resolution of racemic amide, ester or thioester mixtures; and in the synthesis of optically active acids or alcohols from meso-diamides or diesters. Protease active enzymes can also be screened for an ability to synthesize peptides and/or polyesters, e.g., to synthesize, polymerize and/or resolve acid-SCoA esters; to polymerize and depolymerize activated and nonactivated hydroxy esters; and to polymerize and depolymerize activated and nonactivated hydroxy amides (acids). These enzymes can also be screened for an ability to resolve racemic mixtures of amino acid esters; for an ability to synthesize non-natural amino acids. As detergents (e.g., in protein hydrolysis), proteolytic enzymes may be developed, e.g., for the optimization of temperature range and stability; for the optimization of fabric and soil binding properties; for the optimization of stability and/or activity in presence of one or more soils, surfactants, builders, stabilizers, oxidants and chelators used in domestic or industrial detergent formulations; and/or for the enhancement of expression and/or yield of commercial enzyme preparation or the cell expressing such an enzyme, including but not limited to altering the preferred production host to allow for use of less expensive raw materials. Protease may also be screened for an ability to catalyze acylations, alkylations and/or acetylations. Other protease screens might include, e.g., thermostability and/or thermoactivation.

Glycosidases and glycosyl transferases are optionally selected or screened for many different characteristics, e.g., sugar/polymer synthesis; cleavage of glycosidic linkages to form mono, di- and oligosaccharides; synthesis of complex oligosaccharides; glycoside synthesis using UDP-galactosyl transferase; transglycosylation of disaccharides, glycosyl fluorides, aryl galactosides; glycosyl transfer in oligosaccharide synthesis; diastereoselective cleavage of P-glucosylsulfoxides; asymmetric glycosylations; food processing; and paper processing.

Phosphatases and kinases are optionally selected or screened for an ability, e.g., to synthesize/hydrolize phosphate esters (e.g., regio-, enantioselective phosphorylation; the introduction of phosphate esters; the synthesis of phospholipid precursors; and controlled polynucleotide synthesis. They can also be screened, e.g., for an ability to activate biological molecules and/or selective phosphate bond formations without protecting groups.

Mono/Di-oxygenases can be screened or selected for many different properties including, e.g., direct oxyfunctionalization of unactivated organic substrates; hydroxylation of alkanes, aromatics, steroids; epoxidation of alkenes; enantioselective sulphoxidation; regio- and stereoselective Bayer-Villiger oxidations; oxidation of thiophenes, including benzothiophenes, dibenzothiophenes, polycyclic and polyaromatic thiophenes, including coal suspensions and extracts, crude oil fractions, including the middle distillate fractions those derived from it including those with 10-10000 ppm sulfur; enhancement of electron transfer efficiency of the thioredoxin and other components and other polypeptide components of the monooxygenase complex; stabilization and enhancement of mono-/di-oxygenase expression in non-source organisms; and/or stabilization and enhancement of mono-/di-oxygenase stability and performance in solvent, crude oil and mixtures containing them.

Haloperoxidases can be screened for various properties including, e.g., oxidative addition of halide ion to nucleophilic sites; addition of hypohalous acids to olefinic bonds; ring cleavage of cyclopropanes; activated aromatic substrates converted to ortho and para derivatives; 1,3 diketones converted to 2-halo-derivatives; heteroatom oxidation of sulfur and nitrogen containing substrates; and/or oxidation of enol acetates, alkynes and activated aromatic rings.

Lignin peroxidase/Diarylpropane peroxidase can be screened, e.g., for the oxidative cleavage of C—C bonds; the oxidation of benzylic alcohols to aldehydes; the hydroxylation of benzylic carbons; phenol dimerization; hydroxylation of double bonds to form diols; and/or the cleavage of lignin aldehydes.

Epoxide hydrolases can be screened for various abilities, including, e.g., the synthesis of enantiomerically pure bioactive compounds; the regio- and enantioselective hydrolysis of epoxide; the aromatic and olefinic epoxidation by monooxygenases to form epoxides; the resolution of racemic epoxides; and/or the hydrolysis of steroid epoxides

Nitrile hydratase/nitrilase can be screened for different abilities, including, e.g., the hydrolysis of aliphatic nitriles to carboxamides; the hydrolysis of aromatic, heterocyclic, unsaturated aliphatic nitriles to corresponding acids; the hydrolysis of acrylonitrile, adiponitrile and other dinitriles; the production of aromatic and carboxamides, carboxylic acids (nicotinamide, picolinamide, isonicotinamide); the regioselective hydrolysis of acrylic dinitrile; and/or catalyzation of alpha-amino acids from alpha-hydroxynitriles.

Transaminases can be screened for an ability to transfer amino groups to oxo-acids. Amidases/Acylases can be screened for abilities, such as the hydrolysis of amides, amidines, and other C—N bonds and/or the resolution and synthesis non-natural amino acids. Dehalogenase screens can include, e.g., enhanced rates of hydrolysis of polychlorinated alkanes; enhanced stabilities and activities of dichloropropane and trichloropropane hydrolysis; altered specificities toward new substrates; improved stereospecificities of dehalogenase enzymes; and/or improved activity retention during and after immobilization.

Some other general physicochemical properties which can be improved or altered by the instant invention include, e.g., substrate or product specificity; substrate or product spectrum; substrate or product affinity (or Kₘ); inhibitor spectrum and inhibitor properties (or Kᵢ); substrate, product or inhibitor spectrum; metal, cofactor, or prosthetic group requirements, sensitivities and specificities; kinetic constants under standard and specific operational conditions; turnover numbers; maximal and operational reaction velocities; operational temperature optima and ranges; operational pH optima and ranges; oxidative sensitivity; solvent compatibility and stability; salt stability or concentration ranges and optima; surfactant, emulsifier and chelator compatibilities; host-specific expression properties; coordinated improvements in multiple physicochemical properties; relative kinetic performance of soluble, solublized, immobilized, emulsified; and/or, encapsulated, crystallized or differentially prepared enzyme mixtures.

Note, that expression products or hosts expressing those products made by the methods described herein are optionally screened or assayed for multiple traits or properties. For example, a host expressing, e.g., an enzyme produced by the methods of the invention may be screened initially for the efficient catalyzation of a particular reaction of interest, and subsequently screened for stability under shearing conditions or any other property. Any number or combination of desired traits or properties may be screened. Furthermore, in certain embodiments, multiple properties can be screened in a single assay.

Integrated Systems

The present invention also provides computers, computer readable media and integrated systems comprising character strings corresponding to single-stranded nucleic acid templates, chimeric nucleic acid sequences, nucleic acid fragments, and the like. Sequences that can be manipulated in a computer system include upstream and/or downstream sequences that are provided or produced by the methods described herein. In addition, integrated systems can be used to model the recombinational approaches set forth herein. That is, single-stranded templates or fragments are optionally designed in silico. These fragments or templates can then be synthesized and physical recombination can be performed as noted herein. Accordingly, the present invention can use computer-assisted design and synthesis in combination with the other methods herein (or separately from the other methods). In any case, sequences of interest can be manipulated by in silico recombination methods, or by standard sequence alignment (also discussed, supra), word processing software, or the like. A variety of in silico sequence manipulation methods are described, e.g., in Selifonov et al., filed Jan. 18, 2000, (PCT/US00/01202) and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); and “METHODS OF POPULATING DATA STRUCTURES FOR USE IN EVOLUTIONARY SIMULATIONS” by Selifonov and Stemmer, filed Jan. 18, 2000 (PCT/US00/01138).

For example, different types of similarity and considerations of various stringency and character string length can be detected and recognized in the integrated systems herein. For example, many homology determination methods have been designed for comparative analysis of sequences of biopolymers, for spell-checking in word processing, and for data retrieval from various databases. With an understanding of double-helix pair-wise complement interactions among four principal nucleobases in natural polynucleotides, models that simulate annealing of complementary homologous polynucleotide strings can also be used as a foundation of recombination according to the methods herein, sequence alignment or other operations typically performed on the character strings corresponding to the sequences herein (e.g., word-processing manipulations, construction of figures comprising sequence or subsequence character strings, output tables, etc.). An example of a software package which can perfom genetic operations for calculating sequence similarity is BLAST, which can be adapted to the present invention by inputting character strings corresponding to the sequences herein.

As mentioned above, BLAST is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915). Thus, BLAST can be used to align any sequences to be recombined, e.g., to check for any homology parameter of interest.

An additional example of a useful sequence alignment algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS5:151-153 (1989). The program can align, e.g., up to 300 sequences of a maximum length of 5,000 letters. The multiple alignment procedure begins with the pairwise alignment of the two most similar sequences, producing a cluster of two aligned sequences. This cluster can then be aligned to the next most related sequence or cluster of aligned sequences. Two clusters of sequences can be aligned by a simple extension of the pairwise alignment of two individual sequences. The final alignment is achieved by a series of progressive, pairwise alignments. The program can also be used to plot a dendogram or tree representation of clustering relationships. The program is run by designating specific sequences and their amino acid or nucleotide coordinates for regions of sequence comparison. Thus, PILEUP can be used to align any sequences to be recombined, e.g., to check for any homology parameter of interest.

Standard desktop applications such as word processing software (e.g., Microsoft Word™ or Corel WordPerfect™) and database software (e.g., spreadsheet software such as Microsoft Excel™, Corel Quattro Pro™, or database programs such as Microsoft Access™ or Paradox™) can be adapted to the present invention by inputting character strings corresponding to, e.g., single-stranded nucleic acid template sequences, chimeric gene sequences or subsequences thereof, or other nucleic acid sequences. For example, the integrated systems can include the foregoing software having the appropriate character string information, e.g., used in conjunction with a user interface (e.g., a GUI in a standard operating system such as a Windows, Macintosh or LINUX system) to manipulate strings of characters. As noted, specialized alignment programs such as BLAST or PILEUP can also be incorporated into the systems of the invention for alignment of nucleic acids or proteins (or corresponding character strings).

Integrated systems for analysis in the present invention typically include a digital computer with software for aligning or manipulating single-stranded nucleic acid templates, chimeric gene sequences or subsequences thereof, or other nucleic acid sequences, as well as data sets entered into the software system comprising any of the sequences herein. The computer can be, e.g., a PC (Intel x86 or Pentium chip-compatible DOS™, OS2™ WINDOWS™ WINDOWS NT™, WINDOWS95™, WINDOWS98™ LINUX based machine, a MACINTOSH™, Power PC, or a UNIX based (e.g., SUN™ work station) machine) or other commercially common computer which is known to one of skill. Software for aligning or otherwise manipulating sequences is available, or can easily be constructed by one of skill using a standard programming language such as Visual basic, Fortran, Basic, Java, or the like.

Any controller or computer optionally includes a monitor which is often a cathode ray tube (“CRT”) display, a flat panel display (e.g., active matrix liquid crystal display, liquid crystal display), or others. Computer circuitry is often placed in a box which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of single-stranded nucleic acid template sequences, chimeric gene sequences or subsequences thereof, or other nucleic acid sequences to be compared or otherwise manipulated in the relevant computer system.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the system to carry out any desired operation, e.g., nucleic acid sequence alignment, nucleic acid synthesis, etc.

In one aspect, the computer system is used to perform in silico recombination of character strings that correspond to, e.g., chimeric nucleic acid sequences or subsequences, isolated nucleic acid fragment sequences, and the like. A variety of methods that can be adapted to the present invention are set forth in, e.g., in Selifonov et al., filed Jan. 18, 2000, (PCT/US00/01202) and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); and “METHODS OF POPULATING DATA STRUCTURES FOR USE IN EVOLUTIONARY SIMULATIONS” by Selifonov and Stemmer, filed Jan. 18, 2000 (PCT/US00/01138). In addition to performing in silico recombination which models or assists in the present methods, any of the in silico manipulations described in the preceeding references can be performed as upstream or downstream operations, e.g., to provide single-stranded nucleic acids or fragments, or to further modify or otherwise manipulate any product produced by any method herein.

For example, in the references previously noted, genetic operators are used in genetic algorithms to change given sequences, e.g., by mimicking genetic events such as mutation, recombination, death and the like. Multi-dimensional analysis to optimize sequences can also be performed in the computer system, e.g., as described in the '375 application.

A digital system can also instruct an oligonucleotide synthesizer to synthesize single-stranded nucleic acid templates, chimeric gene sequences or subsequences, or other nucleic acid fragment sequences, e.g., used for gene reconstruction or recombination, or to order those sequences from commercial sources (e.g., by printing appropriate order forms or by linking to an order form on the internet).

The digital system can also include output elements for controlling nucleic acid synthesis (e.g., based upon a sequence or an alignment of nucleic acid sequences as herein), i.e., an integrated system of the invention optionally includes an oligonucleotide synthesizer or an oligonucleotide synthesis controller for synthesizing, e.g., single-stranded nucleic acid templates, chimeric gene sequences or subsequences, or other nucleic acid fragment sequences. The system can include other operations which occur downstream from an alignment or other operation performed using a character string corresponding to a sequence herein, e.g., as noted above with reference to assays.

Kits

The present invention also provide a kit for performing the methods of single-stranded nucleic acid template-mediated recombination or nucleic acid fragment isolation described herein. The kit or system can optionally include a set of instructions for practicing one or more of the methods described herein; one or more assay components that can include at least one single-stranded nucleic acid template or nucleic acid sequences, and one or more reagents (e.g., affinity labels, binding agents with linked magnetic beads, and the like); and a container for packaging the set of instructions and the assay components.

### EXAMPLES

The following examples illustrate various aspects of the invention. The examples are not intended to be limiting; one of skill will recognize a variety of non-critical parameters that can be altered while achieving substantially similar results.

I. Single-Stranded Nucleic Acid Template and Nucleic Acid Preparative Approaches

This section illustrates various non-limiting approaches for generating single-stranded nucleic acid templates and nucleic acid fragment populations for use in the methods described herein. The methods for producing single-stranded nucleic acid templates include, e.g., unidirectional nucleic acid amplifications, magnetic-based separations, nuclease-mediated methods, and selective RNA/DNA herteroduplex degradations. In these examples, nucleic acid fragment populations are optionally derived from, e.g., previously isolated single-stranded nucleic acids or uncharacterized environmental nucleic acid fragment isolates, or are directly synthsized.

### Example 1

### Preparation of Single-Stranded Template Subtilisin RC1 Sense DNA

A. Unidirectional “Amplification” of Subtilisin Sense Strand

Subtilisin variants RC1 and RC2 (Zhou et al., (1998) “Regulatory Roles of the P Domain of the Subtilisin-like Prohormone Convertases,” J. Biol. Chem., 273(18):11107) are obtained from the pBE3 Shuttle vector described by Zhao and Arnold (1997) “Functional and nonfunctional mutations distinguished by random recombination of homologous genes,” Proc. Natl. Acad. Sci. U.S.A. 94(15):7997-8000. In this approach, single-stranded sense DNA is obtained by first obtaining the RC1 double stranded DNA by digestion of the RC1-pBE3 construct with BamHI and NdeI, followed by subsequent gel purification of the subtilisin insert. Approximately 50 ng of the insert DNA is subjected to recursive single primer (P3B) extension. DNA extension is conducted at a 30-fold molar excess of the primer to template. Single strand copying and accumulation is mediated by 10 rounds for 30 seconds at 94° C., 30 seconds at 55° C. and 1 minute at 72° C.; plus a 2 minute extension (incubation at 72° C.) following the final round. The single strand product and template DNAs are isolated from other reaction components using the Qiaex PCR clean-up kit (Qiagen, Inc.). Digestion of the mixed population of DNA with Dpn I (or other appropriate restriction endonucleases), followed by gel purification of the >1 kb band results in isolation of a pure population of single-stranded sense subtilisin DNA.

B. Magnetic-Based Separation of Template Strands

In this approach, one of the two primers (P5N and P3B, Zhao et al, 1998, supra) is synthesized with a 5′amino label (e.g., Aminolink, Clontech, Inc., Mountain View, Calif.) and followed by covalent coupling of the labeled oligonucleotide to magnetic high density latex beads (>10 units). In the present example, an amino modified derivative of primer P3B is coupled to a magnetic bead support to give primer Im3B. Amplification (100 μl) in the presence of ImP3B, P5N and the RC1 template is followed by magnetic separation of strands at elevated temperatures, resulting in one strand remaining attached to a solid matrix or surface while the other strand remains in solution as single stranded DNA.

Briefly, about 30 pmol each of the ImP3B and P5N primers are added to a 100 [l amplification mixture containing 1× Taq polymerase buffer (Pro Mega, Madison, Wis.), 0.2 m/m dNTPs, 1.5 mM MgCl₂, and 2.5 units of Taq polymerase (Pro Mega, Madison, Wis.) and ˜1 pg of plasmid DNA followed by 25 cycles of the thermal profile consisting of 30 seconds at 94° C., 30 seconds at 55° C., and 1 minute at 72° C.; plus a 2 minute extension (incubation at 72° C.) following the final round. Following amplification, the amplification mixture is diluted to 0.25 ml with 1× SSC buffer and heated to 99° C. for 10 minutes. Thorough mixing is assured by periodic manual mixing of the capped tube by briefly lifting out of the 99° C. heat block. A small magnet is position just under the tube when it is positioned within the 99° C. heat bath. Magnetic beads are allowed to settle out and adhere to the attractive surface while the solution is removed and transferred to a second tube. The heat denaturation/magnetic separation process is repeated for each of the resulting tubes to assure efficient separation, followed by pooling of the bound populations from the first and second rounds. The unbound fractions are pooled, ethanol precipitated, washed, resuspended and digested briefly with a double stranded DNA-specific, frequent cutting restriction endonuclease (e.g., Dpn I). The intact full-length single-stranded DNA is isolated by gel electrophoresis in a 1% agarose/1× TBE gel and purified using the QiaPrep system (Qiagen). The resulting single-stranded template DNA provides a highly pure template for subsequent recombination. Note, the bound fraction can either be discarded or used, e.g., to generate single-stranded fragment populations. See, Example 2, below.

C. Nuclease-Based Formats for Generating Single-Stranded Templates

Certain exonucleases, such as Exonuclease III, Bal31 and Mung bean nuclease are known to selectively degrade various forms of double stranded or partially double stranded DNA. Each can be used to selectively degrade double stranded nucleic acids such that the strand of interest is preserved. For example, ExoIII will progressively digest double stranded DNA starting from a blunt or recessed 3′ end, but not from a free single-stranded 3′ end. In this example, ExoIII is used to selectively degrade either the upper or lower strand of a nucleic acid duplex in which the non-degraded strand is protected by having a 3′ end that extends beyond the 5′ terminus of the opposite strand.

A modified version of the P5N primer is generated in which the 6 bases encoding the NdeI site (CATAG) are replaced with bases encoding the KpnI restriction site. The Kpn-modified primer is referred to as P5NKpn. Subtilisin DNA is amplified in the presence of P5NKpn and P3B using standard conditions. Following amplification and purification of the amplification product, the product is digested with KpnI to create a 3′ overhang on the bottom strand. Digested and purified DNA is subjected to exonuclease digestion using standard conditions (see, e.g., Ausubel and Sambrook, supra). Subsequent to stopping the reaction, characterization and isolation of the digested DNA via preparative gel electrophoresis results in pure populations of single-stranded RC1 and single-stranded RC2 bottom strand. Purified single stranded DNA corresponding to the upper strand can be generated in a similar manner. Briefly, a KpnI modified version of the P3B primer (P3BKpn) is synthesized and used to amplify RC1 and RC2 templates in conjunction with the unmodified P5N primer. Amplified DNA is digested with KpnI and then with ExoIII.

D. RNA/DNA Heteroduplex Generation as a Way to Create Single-Stranded Templates

In this example, a gene, a pathway, a family or a fragment of a gene is cloned into a vector (e.g., pBluescript, pET series vectors, or the like) enabling easy in vitro trancription of RNA corresponding to the target sequence. Transcripts are generated using one of many commercially available in vitro transcription kits. The transcripts so generated are primed for second strand synthesis with an appropriately positioned oligonucleotide primer and the second strand is synthesized with reverse transcriptase. Reverse transcription provides single-stranded DNA from which the RNA can be selectively degraded using a variety of commercially available RNases (RNase A, RNase H, and the like).

In the instant example, DNA corresponding to subtilisin E RC1 is excised from the pBE vector with restriction enzymes NdeI and BamHI, gel purified, and ligated into appropriately digested pBluescript SK. Clones containing the RC1 insert (pRC1-Blue) are isolated following transformation of the competent E. coli HB101, then plated on LB/agar/100 μg/ml selection plates. One or more clones are selected for further use and inoculated (100 μl) into 0.5 L of LB/Amp (100 μg/ml) and grown to saturation by incubating at 37° C. for 12 hours with vigorous shaking. Plasmid DNA is isolated using the Qiagen MaxiPrep® system according to manufacturer's instruction. Approximately 5 μg is linearized by digestion with BamHI and the resulting plasmid DNA is added to an in vitro transcription mixture generated from the reagents and protocols supplied with the TranScribe kit. Resulting RNA (˜5 μg) is precipitated, and resuspended in RNase-free, sterile water.

Approximately 1 μg of RC1 RNA and 50 ng of P3B oligonucleotide DNA are added to a mixture containing 1× MLV reverse transcription buffer and reaction components (e.g., dNTPs) called for in the MLV transcription reaction (Life Technologies, Inc.). The mixture is heated to 99° C. and allowed to cool slowly over 20 minutes to 37° C. Reverse transcriptase is added and the reaction allowed to proceed for 1 hr at 37° C. The reaction is terminated by heating to 99° C. for 5 minutes followed by addition of one unit of RNase A and incubation at room temperature for 15 minutes. To assure efficient degradation of the RNA, the sample is heated to 99° C. once more and transferred to a 37° C. water bath for an additional 15 minutes. Purified single-stranded DNA is prepared using the PCR product purification kit from Qiagen.

As noted, either RNA or DNA are optionally used as the template strand. However, templating with RNA, in particular, provides an easy route to eliminate template.

### Example 2

### Subtilisin Fragment Preparation

Provided single-stranded nucleic acid templates are used, the instant invention does not require the use of second strand fragment populations derived from single stranded nucleic acids. Rather, the fragment population may be provided by digestion of double stranded (see, Section II, below) or single stranded nucleic acid, such as by DNase or RNase, physical shearing of the same, direct synthesis of either single or double stranded DNA sequences, direct extraction from environmental or uncharacterized biological materials and many other methods. However, fragments derived from single stranded DNA populations do provide for added efficiency and controllability of the recombination process. Of the methods described herein, the packaging of single stranded phagemid (see, Sections II and III, below), selective strand degradation and magnetic separation methods all provide efficient methods for producing single stranded DNA. Such DNA (as well as double stranded DNA) can be randomly or non-randomly fragmented using a wide variety of approaches, including physical, chemical and enzymatic methods.

The following illustrate several non-limiting approaches to template fragmentation.

A. Preparation of Fragment Population from Previously Isolated Single-Stranded Nucleic Acid

In this example, the pelleted beads (Section I) are resuspended in 50 μl of 50 mM Tris-Cl, pH 7.5, 10 mM MnCl₂ (fresh). The suspension is aliquoted into 4 tubes to which has been added 0.1, 0.2, 0.5 or 0.8 μl of 15 units/ml DNase. The tubes are incubated for 10 minutes at room temperature and the reactions stopped by addition of 1 μl 0.5 M EDTA, pH 8.0. To each sample, 2.0 μl of 10× loading dye is added and the samples separated and gel purified on 1.5% agarose/TBE preparative gel as described in Sections II and III, below. Fragment populations may be prepared in this way from a large number of clones and from less well characterized and even uncharacterized (e.g., environmental) DNA samples. The bound fraction is washed by rinsing three times with 250 μl of 95° C. 1× SSC buffer. Rinses are discarded. A third portion of magnetic latex beads is added to the pooled unbound fraction. Magnetic separation is mediated by placing a small magnet at the base of the microcentrifuge tube. The RC1 and RC2 subtilisin genes are amplified in the presence of the single stranded template primers P5N and P3B. Single stranded phagemid DNA corresponding to the sense strand of the RC1 variant of subtilisin E (Zhou et al, 1998, supra) is prepared using supplier protocols and methods well known in the art. Similarly, single stranded DNA corresponding to the antisense strand of the RC1 variant and the RC2 variant are prepared using vectors and subtilisin E variants analogous to those described in Zhou et al, 1998, supra. In one variation, single stranded wild-type subtilisin E sense is prepared in phagemid vector pBluescript SK (Stratagene, La Jolla, Calif.), such as pBluescript and fragments of mutant subtilisin E are prepared by fragmenting mutants 1 or 2, responsible for different degrees of thermostability in subtilisn E mutants. Prepare full-length single stranded version of wild-type subtilisin E. Use DNase I, other restriction enzymes or physical means to fragment amplified mutant 1 and mutant 2 subtilissin E genes to average sizes of <<250 bp. Heat mixture to 99° C. for 10 minutes. Cool to 16° C. over 60-120 minutes. Add Klenow or T4 polymerase, or other non-strand displacing polymerase), and T4 ligase and incubate overnight. Extract, precipitate, digest and clone library DNA as described in Zhou et al, 1998, supra.

B. Preparation of Synthetic Oligonucleotide Fragment Pool

In this example, at least one oligonucleotide is synthesized for use in conjunction with the fragment assembly step. Most typically, several oligonucleotides encoding either known or desired diversity along the length of the template are synthesized in such a way as to cover a substantial portion of the templated strand. Overhanging elements are trimmed by a single strand specific exonucelease. Gaps are filled, typically with a nondisplacing DNA polymerase and the fragments ligated using T4 or T4-like ligase. Single primer extension (as in Section I) is used to generate multiple copies of the ligated strand, following which double stranded DNA is eliminated using specific or non-specific duplex degradation. Nucleases are inactivated and two primer amplification is used to amplify and add appropriate restriction sites to the recombined library contained within the now double-stranded library.

C. Isolation of Uncharacterized DNA Fragments from Environmental and Other Complex Nucleic Acid Extracts

In this example, nucleic acids are obtained from uncharacterized or poorly characterized samples or sources. For a description of such sources see, e.g., Short (1999) U.S. Pat. No. 5,958,672 “PROTEIN ACTIVITY SCREENING OF CLONES HAVING DNA FROM UNCULTIVATED MICROORGANISMS.”

Nucleic acid fragments from such samples are used to prime strand synthesis and recombination along a given single-stranded template or family of single-stranded templates.

Briefly, recombined subtilisin-like proteases are obtained from soil DNA by extracting DNA from a plurality of soil and ground water samples using methods known in the art. Groundwater microbes are concentrated by passing through a 0.2 μm filter at low speed and pressure. Soil microbes are released from soil particles using repeated washings with nonlysing concentration of surface active agents including, e.g., 0.1% Triton X-100 and NP40. Microbes are concentrated on filters as described for groundwater microbes. Filters containing microbes from a plurality of such samples are scraped from the filters using 10 mM Tris-Cl pH7.4, 0.1 mM EDTA. The pooled microbial/debris pellet (˜5 ml) is collected in 4-1.7 ml microcentrifuge tubes and pelleted at low speed (˜3000 rpm) in a tabletop microcentrifuge for 10 minutes. Supernatants are discarded. The pellet is resuspended in a total of 0.5 ml TE and collected in a single 1.7 ml micro-centrifuge tube and repelleted. Supernatant is again discarded and the microbial DNA prepared using bacterial chromosomal DNA isolation kit supplied by Qiagen, Orca labs, or the like.

DNA (double stranded) isolated in this way is subjected to DNase-mediated fragmentation (see, Section 1) to an average size of <100 base pairs and added to single-stranded nucleic acid templates in large mass excess (20:1, or 1 μg extracted fragment library to 50 ng template) to assure template hybridization to rare sequences within the library. In this case, the immobilized ImP3B-derived strand produced and isolated in Section 1, above, is used as the template (˜50 ng) and ˜1 μg of pooled environmental DNA fragments are incubated in 1× T4 polymerase buffer (New England Biolabs) and allowed to undergo primer extension and ligation using, e.g, T4 ligase. Strands are separated as described in Section 1, above, and the soluble fraction (library) is amplified with primers to P5N and P3B to produce a full-length recombined library.

### Example 3

### Detection of Enhanced Subtilisins

A. Colony Visual Screening Method I

Cloning, expression and testing of the subtilisin library is as described in Ness et al. (1999) “DNA Shuffling of subgenomic sequences of subtilisin” Nat. Biotechnol. 17:893-896 by plating initially onto an LB agar plate containing dried milk. Appearance of a clearing zone around a colony is indicative of protease activity. Colonies expressing zone clearing activity were inoculated into liquid cultures and tested for a variety of thermostability and other activity parameters.

B. Colony Visual Screening Method 2

In a second library design and screening strategy, the subtilisin library is ligated just upstream of an in-frame GFP-encoding cistron; such that the GFP signal is observed only if it is downstream of a functional open reading frame. In this approach, transformed E. coli are plated onto antibiotic containing growth plates and colonies containing functional subtilisin open reading frames are detected by visualization under uv light. Those exhibiting fluorescence are picked and grown up in liquid culture for further characterization.

C. In Vitro Kinetic Assay Via Secretory Expression

Transfer of the library to the pBE shuttle vector, followed by transformation into B. subtilis and selection of antibiotic resistant transformants by growth on nutrient-antibiotic plates allows for secretory expression and immediate and direct, on-plate measurement of activity and thermostability screening as reported by Zhou et al. (1998), supra, using the succinyl-ala-ala-pro-phe-p-nitroanilide (s-AAPF-pNa) method of Zhou and Arnold (1997), supra. This assay allows for rapid assessment of the thermostability of the clones derived from the template-based recombination process.

D. In Vitro Kinetic Assay Via Cell Permeabilization

While more cumbersome than secretory expression in B. subtilis, intracellular or periplasmic expression of subtilisin in E. coli and other microorganisms also allows for direct, on-plate assessment of activity and thermostability when coupled with an appropriate cell permeabilizing agent. A long list of cell permeabilizing agents and methods are known in the art. Most commonly, bacterial permeabilizing agents will include one or more of: a detergents (e.g., triton x-100, NP40, and the like), short chain alcohols (e.g., methanol, ethanol, and the like), polymixins (e.g., A, B, etc.) and/or the creation of protoplasts.

E. Results

In recombination experiments using the subtilisin variant RC1 (containing the moderately thermostable N218S mutation) and variant RC2 (containing the moderately thermostable N181D mutation) as sources of fragment populations and/or templates, the thermostabilities and activities of the clones are compared with respect to the two parents. Clones are also observed which exhibit normal activity but lower thermostability (e.g., wild-type activity) than the RC1 and RC2 parents or enhanced thermostability versus the two parents arise in part from effective sequence recombination between the RC1/2 parents.

II. Green Fluorescence Protein Illustrates Template-Based Recombination with Single-Stranded Phagemid-Based Recombination and PCR Amplified GFP Fragments

A family of green fluorescent protein (GFP3) mutants has been developed consisting of GFP3 (Crameri et al. (1996) “Improved Green Fluorescent Protein by Evolution Using DNA Shuffling,” Nat. Biotechnol., 14(3):315-319), STOP1 (Tyr40 TAA) and STOP2 (Ser203 TAA). The latter two contain in-frame stop codons which prevent expression of an active GFP protein. When properly expressed in an appropriate host, and when irradiated at ˜390 nm, GFP emits a characteristic green fluorescence making it easy to observe colonies or cells containing it. Its ease of detection, quantum efficiency and compatibility with hosts from three distinct kingdoms of living organisms makes GFP a particularly attractive protein for potential use in in vitro and in vivo diagnostics. GFP has also proven an important initial target for development of improved tools useful for enhancing performance of industrial proteins, therapeutics and other biological and protein products. GFP sequences were modified as noted below.

### Example 4

### Preparation of Single Stranded Template

a. Single stranded GFP3STOP1 phagemid DNA was prepared by streaking E. coli strains MG108 μM522 proAB/F′ proAB+] and MG122 [MG108+pBAD(Cm)GFP(c3)STOP1 (5812 bp) onto agar plates containing minimal glucose media+thiamine to maintain F′ episome. Plates were incubated overnight at 37° C.

b. Isolated colonies of MG108 and MG122 were each inoculated into 3 ml 2× YT and 2×YT+30 μg/ml chloramphenicol (2×YT30 Cm) broth, respectively, and incubated with shaking for ˜8 hr at 37° C.

c. 7 tubes containing 3 ml 2×YT and 75 ul of MG108 and each of 7 tubes containing 3 ml 2×YT30 Cm and 75 p] of MG122, were infected with either 100, 50, 25, 10, 5, 1 or 0 μl of helper phage VCSM13 (˜1012 pfu/ml, Strategene). These were incubated with vigorous shaking at 37° C. for ˜16 hours.

d. 1.5 ml of each culture was transferred into a microcentrifuge tube and the cells pelleted by centrifugation.

e. 1.3 ml supernatant were transferred to a fresh 1.5 ml tube and 200 μl of 20% polyethylene glycol (PEG) 8000/2.5 M NaCl solution was added. This was Incubated at room temperature for 15 minutes and the phage pelleted by microcentrifugation at maximum speed for 15 minutes.

f. the supernatant was discarded, with residual supernatant spun down and discarded. The phage pellet was suspended in 50 μl TE buffer.

g. 50 μl phenol (equilibrated with TE, pH 7.4) was added and vortexed. The mixture was centrifuged for two minutes in a microcentrifuge to facilitate phase separation.

h. The aqueous phase was transferred to a 1.5 ml tube containing 300 μl of a 25:1 mixture of 100% ethanol and 3M sodium acetate, pH 5.2. The components were mixed and incubated at room temperature for 15 minutes.

i. Phage DNA was pelleted by microcentrifugation at maximum speed for 15 minutes, washed with 0.5 ml 70% ethanol, repelleted, and dried. Dry phagemid DNA pellet was suspended in 50 μl TE.

### Example 5-Preparation of Defined PCR-Derived GFP Fragments

While this example typically uses doubles stranded DNA as its source of the DNA fragment population, such DNA may equally well be prepared from single stranded phagemid DNA prepared as described above from the opposite strand as that prepared above, and fragmented by physical or enzymatic means. However, the ability to use double stranded DNA populations as sources of fragments introduces versatility into the technique by allowing both in vitro, in vivo and synthetic methods of DNA preparation to be used. In preparative methods involving amplification or other use of synthetic primers, it is advantageous to prepare phosphorylated primers when subsequent high efficiency ligation is desired.

a. Oligonucleotide primers PBADGFP3 (P-ATAAGATTAGCGGATCCTAC) and PBADGFP4 (P-TCGGGCATGGCACTCTTGAA)—which flank the random stop sites in pBAD(Cm)GFP(c3)STOP1 (e.g., ‘STOP1 phagemid’)—were phosphorylated and used to prime amplification of corresponding 500 base pair fragments from the STOP1 and STOP2 phagemids using the TthXL thermostable polymerase mix according to manufacturer's protocol.

b. A unique HindIII restriction site in the STOP2 fragment was used to confirm the difference of sequence between the two amplified fragment populations.

### Example 6

### Annealing and Extension Using Amplified GFP Fragments

a. In this step, a high template:fragment molar ratio (˜25:1) was used to assure “capture” of the available fragments by the template strand. Briefly, ˜2 μg of the single-stranded STOP1 phagemid DNA and ˜4 μg of the STOP1 or STOP2 amplification products were co-precipitated in ethanol, washed with 70% ethanol and suspended in 40 μl PE1 buffer (20 mM Tris-Cl, pH7.5; 10 mM MgCl₂; 50 mM NaCl; 1 mM DTT). The STOP1 and STOP2 mixtures were divided into two 20 μl aliquots (0.5 ml tubes).

b. Tubes containing the DNA solutions were heated to 99° C. for 2.5 minutes and cooled to room temperature over 20 minutes using a thermal cycler. To one each of the STOP1 and STOP2 reaction mixtures were added 201 μl of PE2 buffer (20 mM, Tris-Cl, pH7.5; 10 mM MgCl₂; 1 mM DTT) containing 1 mM ATP and 0.2 mM dNTPs. To the other tube in each set was added 20 μl of the same mixture but with the addition of 10 Weiss units of T4 DNA ligase and 5 units of Klenow to each tube. All four tubes were incubated overnight at 16° C.

c. 1 μl of each mix prepared in step b were mixed with E coli strain MG109 (mutS::Tn5) prepared for electroporation. Strains were electroporated using methods well known in the art. Cells were resuspended in 0.95 ml of SOC medium and incubated for 1 hour at 30° C. with shaking. Ten-fold dilutions ranging from 1/10 to 1:10,000 were plated on agar plates containing 0.2% arabinose, 30 μg/ml chloramphenicol. Incubate overnight at 30° C. Score frequency of GFP+clones by Illumination under UV light.

### Example 7

### Detection of GFP Recombination Indicates Template-Directed Method with PCR Fragments is a High Efficiency Recombination Strategy

Addition of GFP fragments generated by amplification of GFP genes with STOP1 and STOP2-specific oligonucleotides to single-stranded GFP(c3)STOP1 DNA was effective at facilitating recombination of the STOP1 and STOP2 phenotypes. Results were as indicated in Table 1:

TABLE 1

| | GFP+/Cm^{r} Transformants | | | |
|---|---|---|---|---|
| | pBAD(Cm)GFP(c3) | | PBAD(Cm)GFP(c3) | |
| Dilution | STOP1 + STOP1 | | STOP1 + STOP2 | |
| Plated | −Enzymes^{a} | +Enzymes^{a} | −Enzymes^{a} | +Enzymes^{a} |
| 1/10 | 0/˜200 | 1/*^{b} | 4/200 | *^{b}/*^{b} |
| 1/100 | 0/26 | 0/˜1000 | 1/33 | ˜500/˜1000 |
| 1/1,000 | 0/4 | 0/201 | 0/4 | 108/219 |
| 1/10,000 | 0/0 | 0/18 | 0/1 | 14/32 |

^{a}T4 DNA Ligase and Klenow.
^{b}Too many to count.

II. Green Fluorescence Protein Illustrates Template-Based Recombination Using Single-Stranded Phagemid and Random Double Stranded Fragments from GFP(Ap)STOP1 and GFP(Ap)STOP2

Effective recombination of GFP(c3)STOP1 and GFP(c3)STOP2 was also mediated by preparation of single-stranded GFP(c3)STOP1 DNA by the method generally described in the previous example. Fragments of GFP(c3)STOP2 were prepared from double stranded pBAD(Ap)GFP(c3)STOP2 DNA by DNase-catalyzed fragmentation.

### Example 8

### Preparation of Single-Stranded Phagemid Templates

a. Single stranded pBAD(Ap)GFP(c3)STOP1 phagemid DNA was prepared by streaking E. coli strain MG108 [M522 proAB/F′ proAB+] containing pBAD(Ap)GFP(c3)STOP1 (5812 bp) onto agar plates containing minimal glucose media+thiamine to maintain F′ episome. Plates were incubated overnight at 37° C. See Guzman et al. (1995) “Tight regulation, modulation, and high-level expression by vectors containing the arabinose PBAD promoter” J. Bacteriol. 177(14):4121-4130. For details about expression vector pBAD18 and the construction of phagemid pBAD(Ap)GFP(c3)) see Crameri et al., (1996) “Improved green fluorescent protein by molecular evolution using DNA shuffling” Nat. Biotechnol. 14(3):315-319.

b. Isolated colonies of MG108 μM522 proAB/F′ proAB+)/pBAD(Ap)GFP(c3)STOP1 were each inoculated into 3 ml 2×YT 100 μg/ml ampicillin (2×YT100Ap) broth, respectively and incubated with shaking for ˜8 hr at 37° C.

c. To each of 7 tubes containing 3 ml 2×YT and 75 μl of MG108 [NM522 proAB/F′ proAB+]/pBAD(Ap)GFP(c3)STOP1 were added 100, 50, 25, 10, 5, 1 or 0 μl of helper phage VCSM13 (˜1012 pfu/ml, Strategene). These were incubated with vigorous shaking at 37° C. for ˜16 hours.

d. 1.5 ml of each culture were transferred into a microcentrifuge tube and pelleted by centrifugation.

e. 1.3 ml supernatant was transferred to a fresh 1.5 ml tube and add 200 μl of 20% polyethylene glycol (PEG) 8000/2.5 M NaCl solution. This was incubated at room temperature for 15 minutes and pellet phage by microcentrifugation at maximum speed for 15 minutes.

f. The supernatant was discarded, spun down and excess supernatant discarded as well. The phage pellet was suspended in 501 μl TE buffer.

g. 50 μl phenol (equilibrated with TE, pH 7.4) was added and the mixture vortexed. The resulting mixture was centrifuged for two minutes in a microcentrifuge to facilitate phase separation.

h. The aqueous phase was transferred to a 1.5 ml tube containing 300 μl of a 25:1 mixture of 100% ethanol and 3M sodium acetate, pH5.2. Components were mixed and incubated at room temperature for 15 minutes.

i. Phage DNA was pelleted by microcentrifugation at maximum speed for 15 minutes, washed with 0.5 ml 70% ethanol, repelleted and dried. Dry phagemid DNA pellet was suspended in 50 μl TE.

j. Presence of single stranded phagemid DNA was confirmed by electrophoretic separation and visualization of 5 μl of the sample in a 0.7% agarose/TBE gel.

### Example 9

### Preparation of Random Double-Stranded GFP Fragment Pool

While this example uses double stranded DNA as its source of the DNA fragment population, such DNA may equally well be prepared from single stranded phagemid DNA prepared as described above from the opposite strand as that prepared in Section 1, above, and fragmented by physical or enzymatic means. However, the ability to use double stranded DNA populations as sources of fragments introduces versatility into the technique by allowing both in vitro, in vivo and synthetic methods of DNA preparation to be used. In preparative methods involving amplification or other use of synthetic primers, it will be advantageous to prepare phosphorylated primers when subsequent high efficiency ligation is required.

a. Double stranded pBAD(Ap)GFP(c3)STOP2 was prepared using the Qiagen Maxi plasmid isolation kit.

b. Trial fragmentation reactions (n=5) containing ˜2 μg of pBAD(Ap)GFP(c3)STOP2 in 20 μl of 50 mM Tris-C, pH 7.5; 10 mM MnCl₂ (freshly prepared) were prepared.

c. 0. 0.1, 0.2, 0.5 or 0.8 ml of DNaseI was added to each of the 5 tubes. This was mixed and incubated for 10 minutes at room temperature.

d. The DNase digestion was stopped by the addition of 1 μl of 0.5 M EDTA, pH 8.0 and placing on ice. Five microliters of loading buffer was added and reactions were run on 1.5% agarose/TBE preparative gel along with appropriate markers of 100-1000 bp. Reactions conditions yielded ˜50-500 bp fragments in size. Twenty micrograms of pBAD(Ap)GFP(c3)STOP2 was digested for 10 minutes using the selected dilution.

e. Following digestion, the reaction was stopped by addition of EDTA and the fragments were separated by electrophoresis through a 0.7% agarose/1×TBE preparative gel. Fragments of ˜50-500 bp were gel isolated and purified using the Whatman glass microfibre filter paper and dialysis membrane.

f. Fragments were subjected to three phenol extractions and ethanol precipitated, washed in 70% EtOH and air dried. DNA was resuspended in 20 μl TE (˜1 μg).

### Example 10

### Annealing and Extension Using Double-Stranded Fragments Derived from DNase Fragmentation of Templates

a. Aliquots (10 ul; ˜0.5 ug) of the single stranded pBAD(Ap)GFP(c3)STOP1 DNA were added to each of four 0.5 ml microcentrifuge tubes. To each of these was added 10, 5, 1 or 0 ul of the DNA fragment solution prepared in section 2 (above) to give ˜20:1, 10:1, 2:1 and 0:1 fragment to phagemid ratios. The phagemid/fragment DNA solution was precipitated with ethanol, washed with 70% ethanol and suspended in 10 μl PE1 buffer (20 mM Tris-Cl, pH 7.5; 10 mM MgCl₂; 50 mM NaCl; 1 mM DTT).

b. Tubes containing the DNA solutions were heated to 99° C. for 2.5 minutes and cooled to room temperature over a 20 minute period using a thermal cycler. To one each of the STOP1 and STOP2 reaction mixtures were added 20 μl of PE2 buffer (20 mM Tris-Cl, pH7.5; 10 mM MgCl2; 1 mM DTT) containing 1 mM ATP and 0.2 mM dNTPs. To the other tube in each set was added 20 μl of the same mixture but with the addition of 10 Weiss units of T4 DNA ligase and 5 units of Klenow to each tube. All four tubes were incubated overnight at 16° C.

c. 1 μl of each mix prepared in step b were mixed with E coli strain MG109 (NM522 mutS::Tn5) prepared for electroporation. Strains were electroporated using methods well known in the art. Cells were resuspended in 0.95 ml of SOC medium and incubated for 1 hour at 30° C. with shaking. Ten-fold dilutions ranging from 1:10 to 1:10,000 were plated on agar plates containing 0.2% arabinose, 100 μg/ml ampicillin. Incubate overnight at 30° C. Recombination was characterized by scoring the frequency of GFP+ clones by illumination under UV light.

### Example 11

### Detection of GFP Recombination Indicates Template-Directed Method with Random Double-Stranded Fragments

The results from Example 10 are as indicated in Table 2, as follows:

TABLE 2

| | GFP+/Ap^{r} Transformants | | | |
|---|---|---|---|---|
| | Fragments to Phagemid (weight/weight Ratio) | | | |
| Dilution | | | | No |
| Plated | ˜20:1 | ˜10:1 | ˜2:1 | Fragments |
| 1/10 | 29/˜2000 | 29/˜3000 | ˜138/˜4000 | 0/8 |
| 1/100 | 6/˜400 | 3/˜500 | 6/˜500 | 0/4 |
| 1/1,000 | 0/48 | 0/62 | 0/77 | 0/1 |
| 1/10,000 | 0/4 | 0/7 | 1/8 | 0/0 |

These results indicate that the addition of STOP2-specific oligonucleotides to single-stranded GFP(c3)STOP1 DNA is effective at catalyzing recombination of the STOP1 and STOP2 phenotypes.

III. Template-Based Recombination of a Partial Viral Genome Using Single-Stranded Templates, a Strand Non-Displacing Polymerase and Single-Stranded Fragments

### Example 12

### Preparation of Single-Stranded Adenovirus DNA Fragments Using Phagemid Vector

PCR fragments amplified from Adenovirus Ad1, Ad2, Ad5, and Ad6 serotypes were ligated into phage pGEM-T (Promega) via a T-A cloning protocol (see, e.g., phagemid pGEM-T literature and Zhou et al., Biotechniques 19:34-35 (1995) for details regarding similar cloning methods). In this way phagemid derivatives bearing the Adenovirus fragment in either orientations (sense or antisense) with respect to the F1 origin of replication were generated.

Phagemid pGEMT-Ad5 (−) was chosen as source of single strand DNA template and phagemids pGEMT-Ad1-8-4 (+) pGEMT-Ad2-8-3 (+), pGEMT-Ad2-10-2 (+), and pGEMT-Ad6-10-12 (+) were chosen as source of single strand DNA to generate fragments which are complementary to the Ad5 template. Single-strand DNA was prepared from sense and antisense derivatives by infecting cultures bearing the phagemids with helper phage VCSM13 (Strategene) at a moi of ˜10 according to supplier's protocol.

The resulting preparations of single-strand phagemid DNA were digested with restriction endocuclease AluI (New England Biolabs, Inc.) according to manufacturer's protocol. This digestion allows removal of unwanted double-strand phagemid DNA from the samples and prevents the double-stranded phagemid DNA from acting to reassemble parental sequences.

The Ad1, Ad2, Ad5 and Ad6 sense strand derivatives were then fragmented with Dnase I, as discussed above, and ˜25-75 bp fragments were gel-purified, phenol-chloroform extracted, and ethanol precipitated.

### Example 13

### Assembly of Recombined Partial Adenovirus Genomes Using Single-Stranded Fragments and Phagemid Templates

Fragments from the 4 sense strand derivatives were mixed with the antisense strand template at fragment-template molar ratios of 10, 50, and 250. The fragment sense template mixtures were heated at 95° C. for 3 minutes and gradually cooled to room temperature to allow annealing of single strand fragments to the single strand template.

Addition of dNTPs, T4 DNA Polymerase, and T4 DNA Ligase to the fragment sense template mix followed by an ˜2 hour incubation at 37° C. was used to extend and ligate the fragments over the template to generate chimeric DNA molecules between the various Adenovirus serotypes. The resulting extension ligation mix was transformed into an Escherichia coli mutS strain which is defective in mismatch repair to enrich for chimeric clones.

### Example 14

### Recombination of Folding Domains Among Otherwise Low Homology Proteins

In this example, amino acid sequences derived from known or suspected genes and genetic pathways are subjected to at least one of several secondary structure prediction algorithms, sequences are then aligned with other sequences projected to assume the same structure fold. Using the structurally optimized alignment, bridging oligonucleotides are synthesized which will enable otherwise unlikely recombination events to occur between one or more folding elements (strands, helices, loops, etc . . . ) in a plurality of structurally analogous parental genes.

While the foregoing invention has been described in some detail for purposes of clarity and understanding it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

1-43. (canceled)

44. A method of isolating nucleic acid fragments from a set of nucleic acid fragments, the method comprising:
hybridizing at least two sets of nucleic acids, wherein a first set of nucleic acids comprises single-stranded nucleic acid templates and a second set of nucleic acids comprises at least one set of nucleic acid fragments;
separating the hybridized nucleic acids from unhybridized nucleic acids by at least one first separation technique; and
denaturing the separated hybridized nucleic acids to yield the single-stranded nucleic acid templates and isolated nucleic acid fragments.

45-74. (canceled)

75. A method of generating chimeric nucleic acids, the method comprising:
hybridizing a first plurality of first parental single-stranded nucleic acids and a second plurality of second parental single-stranded nucleic acids, wherein the hybridized first and second parental single-stranded nucleic acids comprise at least one nonhybridized region of sequence diversity;
nicking at least one strand in the at least one nonhybridized region of sequence diversity;
cleaving the at least one nicked strand in the at least one nonhybridized region of sequence diversity to provide at least one sequence gap between hybridized regions; and,
elongating, ligating, or both, the at least one sequence gap between the hybridized regions to generate chimeric progeny nucleic acids.

76. The method of claim 75, wherein at least one of the elongating and ligating steps is conducted in vivo.

77. The method of claim 75, wherein at least one of the elongating and ligating steps is conducted in vitro

78-82. (canceled)

83. The method of claim 75, comprising providing the first or second parental single-stranded nucleic acids by performing one or more cycles of an asymmetric polymerase chain reaction.

84. The method of claim 75, comprising providing the first or second parental single-stranded nucleic acids by degrading specific single strands in double-stranded parental sequences with at least one nuclease.

85. (canceled)

86. The method of claim 75, comprising synthesizing the first or second parental single-stranded nucleic acids.

87. The method of claim 86, further comprising randomly or nonrandomly incorporating dUTP into the first or second parental single-stranded nucleic acids during synthesis.

88-93. (canceled)

94. The method of claim 92, wherein the at least one nuclease comprises a Mung bean nuclease or a nickase.

95. The method of claim 75, the cleaving step comprising cleaving the at least one nicked strand in the at least one nonhybridized region of sequence diversity with at least one nuclease.

96-110. (canceled)

111. A method of recombining a set of nucleic acid fragments, the method comprising:
hybridizing at least two sets of nucleic acids, wherein a first set of nucleic acids comprises single-stranded sense strand-nucleic acid templates and a second set of nucleic acids consists essentially of single-stranded antisense strand-nucleic acid fragments; and,
elongating, ligating, or both, sequence gaps between the hybridized nucleic acid fragments to generate at least substantially full-length chimeric nucleic acid sequences that correspond to the single-stranded nucleic acid templates, thereby recombining the set of nucleic acid fragments.

112-115. (canceled)

116. A method of recombining a set of nucleic acid fragments, the method comprising:
providing a set of at least partially double-stranded nucleic acids that encode a polypeptide of interest or portion thereof;
contacting the set of at least partially double-stranded nucleic acids with an exonuclease that selectively degrades one strand of the at least partially double-stranded nucleic acids to provide a set of single-stranded nucleic acid templates;
hybridizing the set of single-stranded nucleic acid templates with a second set of nucleic acids comprising at least one set of nucleic acid fragments; and,
elongating, ligating, or both, sequence gaps between the hybridized nucleic acid fragments to generate at least substantially full-length chimeric nucleic acid sequences that correspond to the single-stranded nucleic acid templates, thereby recombining the set of nucleic acid fragments.

117. The method of claim 116, wherein the exonuclease is selected from the group consisting of Exonuclease II, Bal31, Mung bean nuclease, T7 gene 6 exonuclease, and lambda exonuclease.

118. The method of claim 116, wherein the nucleic acid fragments are single stranded.

119-166. (canceled)
